(19) United States
(12) Patent Application Publication
(10) Pub. No.: US 2024/0400659 A1
(43) Pub. Date: Dec. 5, 2024

(54) CONDITIONALLY ACTIVE PROTEINS FOR NEURODEGENERATIVE DISEASES

(71) Applicant: BioAtla, Inc., San Diego, CA (US)

(72) Inventors: Jay M. Short, Jackson, WY (US); Christina Wheeler, San Diego, CA (US); Jing Wang, San Diego, CA (US); Matthew Lucas, San Diego, CA (US); Haizhen Liu, San Diego, CA (US); Hwai Wen Chang, San Marcos, CA (US); Gerhard Frey, San Diego, CA (US)

(73) Assignee: BioAtla, Inc., San Diego, CA (US)

(21) Appl. No.: 18/694,273

(22) PCT Filed: Oct. 17, 2022
(86) PCT No.: PCT/US2022/078200

§ 371 Date: Mar. 21, 2024

Related U.S. Application Data
(60) Provisional application No. 63/257,373, filed on Oct. 19, 2021.

(51) Int. Cl. C07K 16/18; A61K 45/06; A61K 47/64

(52) U.S. Cl. CPC C07K 16/18; A61K 45/06; A61K 47/64; C07K 2317/56

(57) ABSTRACT

A polypeptide or conditionally active protein suitable for prevention or treatment of a neurodegenerative disease that binds to ApoE. The conditionally active protein binds to ApoE with an increased binding activity at an aberrant condition such as an acidic pH in a dementia brain in comparison with the binding activity to ApoE at a normal physiological condition such as the pH of blood. A method for generating the conditionally active protein is also provided.

### CROSS-REFERENCE TO RELATED APPLICATIONS

This application claims the benefit of U.S. provisional application No. 63/257,373, filed on Oct. 19, 2021, the disclosure of which is hereby incorporated by reference in its entirety.

### INCORPORATION OF MATERIAL OF XML SEQUENCE LISTING BY REFERENCE

The sequence listing submitted herewith as an XML file named “BIAT1024WO_Sequence_Listing_XML” created Oct. 12, 2022, which is 58,000 bytes in size, is hereby incorporated by reference in its entirety.

### FIELD OF THE DISCLOSURE

This disclosure relates to the field of treating neurodegenerative diseases. Particularly, this disclosure relates to conditionally active proteins for treating neurodegenerative diseases and methods of generating such conditionally active proteins.

### BACKGROUND OF THE DISCLOSURE

Neurodegenerative diseases are associated with loss of neuronal function and structure, and even neuron death. Neurodenerative disease may lead to movement disorders, such as ballism, ataxia, hyperkinesis, Parkinsons, athetosis, chorea, and dyskinesias, as well as neuropsychiatric symptoms. In particular, Huntington's Disease (HD), Parkinson's Disease (PD), and Alzheimer's Disease (AD) may have symptoms including movement disorders or dysfunctions, in addition to neuropsychiatric disorders, such as aggression, irritability, and anxiety.

The Apolipoprotein E (ApoE) locus in the human genome is strongly associated with the risk of developing neurodegenerative diseases. For example, ApoE is best known for being linked with AD, among the neurodegenerative diseases. Genetic studies have revealed allelic linkage of the ApoE gene to families with a higher risk of late onset AD and to sporadic AD. There are three major alleles of ApoE, termed Apolipoprotein E2 (ApoE2, SEQ ID NO:1), Apolipoprotein E3 (ApoE3, SEQ ID NO:2), and Apolipoprotein E4 (ApoE4, SEQ ID NO:3), of which ApoE4 is the AD risk factor. The frequency of ApoE4 is 65% in non-related patients with sporadic AD and 80% in those with familial AD. ApoE4 may also increase the risk for AD by lowering the age of onset of the disease by 7 to 9 years per allele copy. Declining memory and brain pathology have been reported in middle-aged and young ApoE4 carriers with ongoing normal clinical status, suggesting that the effects of ApoE4 start decades before the onset of AD. Interestingly, ApoE2 is protective in this regard as it decreases the probability of developing AD, while ApoE3 appears to be neutral in terms of AD risk.

ApoE is a fat-binding protein that is part of the chylomicron and intermediate-density lipoprotein (IDLs), which are essential for the normal catabolism of triglyceride-rich lipoproteins. In peripheral tissues, ApoE is primarily produced by the liver and macrophages for mediating cholesterol metabolism. In the central nervous system, ApoE is mainly produced by astrocytes for transporting cholesterol to neurons via ApoE receptors, which are members of the low density lipoprotein receptor gene family. ApoE is the principal cholesterol carrier in the brain for delivery of cholesterol and phospholipids to neurons. The cholesterol and lipids delivered by ApoE are important building blocks for the formation of neuron membrane, terminal and synapto-dendritic structures in brains.

Research consistently shows that people who have high total cholesterol levels in blood during midlife are on average more likely to develop dementia compared to those with normal total cholesterol level. In a review of eight studies with a total of over 14,000 participants, it was shown that high total cholesterol level in the blood measured at midlife was significantly associated with an increased risk of Alzheimer's disease and dementia late in life (Anstey, et al., “Cholesterol as a risk factor for dementia and cognitive decline: A systematic review of prospective studies with meta-analysis,” American Journal of Geriatric Psychiatry, vol. 16, pp. 343-354, 2008). Some studies have shown that use of statins to lower blood cholesterol can also lower the risk of dementia.

Pathologically, ApoE4 is associated with increased deposition of amyloid R in the brain (amyloid plaques), impaired neuronal plasticity, and increased neuropathology. Methods and therapeutic agents targeting ApoE4 have been developed for treatment of dementia. US 2012/0009125 discloses methods and kits for prophylactically treating a patient at risk for or suffering from dementia. The methods include steps of (1) determining the patient's genotype of genes related to susceptibility for dementia, and particularly Alzheimer's; and (2) characterizing the patient as having enhanced risk of amyloid production and/or impaired amyloid clearance (e.g., positive for ApoE4 and/or clustering). The patient may be treated with therapeutic agents which induce heat shock proteins and/or agents which modulate GLT-1 activity/expression by astrocytes.

US 2013/0017251 discloses a recombinant antibody that specifically binds to an epitope in ApoE comprising amino acid residues within amino acids 222-230 and 261-272 of ApoE. The recombinant antibody comprises complementarity determining regions (CDRs) from the 3H1 light chain variable region sequence and CDRs from the 3H1 heavy chain variable region sequence. US 2015/0337030 provides a neuron model that can be used to screen and identify compounds that may prevent ApoE4-induced late onset AD in patients. Further, methods are also provided for the treatment and/or prevention of a neurodegenerative disorder by using an inhibitor of ApoE4, such as an antibody, peptide, peptidomimetic, and antisense RNA.

US 2015/0118231 discloses methods and compositions for treating or preventing a neurodegenerative condition by systemic administration of specific antibodies against ApoE4 to a subject in need thereof. The neurodegenerative condition may be caused by a disease, such as Alzheimer's disease, or be induced by various head or brain injuries. The ApoE4 antibody is a monoclonal antibody specifically recognizing, interacting with and/or binding the ApoE4 protein.

One drawback of the antibodies and peptides that target ApoE is that they are as active in normal tissues as they are in the brain where the treatment is needed. Inhibition of ApoE in normal tissues where ApoE has an important normal physiological function in lipid transportation and cholesterol metabolism inevitably hinders normal cholesterol metabolism and can potentially cause serious side-effects.

The present invention addresses this problem by providing conditionally active proteins that are more active in inhibition of ApoE in the dementia brain, especially where amyloid plaques exist or are being formed, in comparison to the level of inhibition of ApoE in a normal tissue or organ. The conditionally active proteins of the present invention can thus be used to provide targeted treatment for dementia in combination with a reduced interference with normal cholesterol metabolism in normal tissue or organs of ApoE. In addition, the conditionally active proteins of the present invention provide a mechanism to facilitate their crossing of the blood brain barrier. Thus, these conditionally active proteins are significantly more likely to be delivered to the dementia brain to provide treatment of neurodegenerative diseases.

### SUMMARY OF THE DISCLOSURE

In one aspect, the disclosure provides an isolated polypeptide comprising:
a heavy chain variable region having three complementarity determining regions having H1, H2, and H3 amino acid sequences, wherein:
(a) the H1 amino acid sequence is GYTFTTAGX₁Q (SEQ ID NO: 31),
(b) the H2 amino acid sequence is WX₂NTHSGEPKYAEDFKG (SEQ ID NO: 32), and
(c) the H3 amino acid sequence is X₃GGYAX₄DY (SEQ ID NO: 33); wherein X₁ is M or D,
X₂ is I or D,
X₃ is M or E, and
X₄ is M or E; and
a light chain variable region having three complementarity determining regions having L1, L2, and L3 amino acid sequences, wherein:
(d) the L1 amino acid sequence is KASEDINSYLS (SEQ ID NO: 34),
(e) the L2 amino acid sequence is RANRLVD (SEQ ID NO: 35), and
(f) the L3 amino acid sequence is LQX₅DEFX₆LT (SEQ ID NO: 36);
wherein X₅ is Y or D and X₆ is P or D;
with the proviso that X₁, X₂, X₃, X₄, X₅ and X₆ cannot be M, I, M, M, Y, P, respectively, at the same time.

In the embodiment of paragraph [0008], the H1 sequence may be selected from SEQ ID NOs: 37 and 38, the H2 sequence may be selected from SEQ ID NOs: 39 and 40, and the H3 sequence may be selected from SEQ ID NOs: 44-46.

In any one of the embodiments of paragraphs [0008]-[0009], the heavy chain variable region may be:
the heavy chain variable region having the H1 sequence of SEQ ID NO: 37, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 40, and the H3 sequence of SEQ ID NO: 44;
the heavy chain variable region having the H1 sequence of SEQ ID NO: 38, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 39, and the H3 sequence of SEQ ID NO: 45; and
the heavy chain variable region having the H1 sequence of SEQ ID NO: 37, the H2 sequence of SEQ ID NO: 39, and the H3 sequence of SEQ ID NO: 46.

In the embodiment of paragraph [0008], the heavy chain variable region of the isolated polypeptide above may have an amino acid sequence selected from the amino acid sequences of SEQ ID NOs: 22-27.

In any one of the embodiments of paragraphs [0008]-[0011], the light chain variable region may have an amino acid sequence selected from the amino acid sequences of SEQ ID NOs: 28-30.

In any one of the embodiments of paragraphs [0008]-[0009], the L1 sequence may be SEQ ID NO: 34, the L2 sequence may be SEQ ID NO: 35, and the L3 sequence may be selected from SEQ ID NOs: 41-43.

In the embodiment of paragraph [0010], the L1 sequence may be SEQ ID NO: 34, the L2 sequence may be SEQ ID NO: 35, and the L3 sequence may be selected from SEQ ID NOs: 42-43.

In the embodiment of paragraph [0008], the L1 sequence may be SEQ ID NO: 34, the L2 sequence may be SEQ ID NO: 35, and the L3 sequence may be SEQ ID NO: 41; and
the heavy chain variable region may be selected from:
the heavy chain variable region having the H1 sequence of SEQ ID NO: 37, the H2 sequence of SEQ ID NO: 40, and the H3 sequence of SEQ ID NO: 44;
the heavy chain variable region having the H1 sequence of SEQ ID NO: 38, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 39, and the H3 sequence of SEQ ID NO: 45; and
the heavy chain variable region having the H1 sequence of SEQ ID NO: 37, the H2 sequence of SEQ ID NO: 39, and the H3 sequence of SEQ ID NO: 46.

In the embodiment of paragraph [0008], the heavy chain variable region may have the H1 sequence of SEQ ID NO: 37, the H2 sequence of SEQ ID NO: 39, and the H3 sequence of SEQ ID NO: 44; and the light chain variable region may have the L1 sequence of SEQ ID NO: 34, the L2 sequence of SEQ ID NO: 35, and the L3 sequence selected from SEQ ID NOs: 42-43.

In the embodiment of paragraph [0008], the heavy chain variable region may have an amino acid sequence of SEQ ID NO: 22; and the light chain variable region may have an amino acid sequence selected from the amino acid sequences of SEQ ID NOs: 29-30.

In the embodiment of paragraph [0008], the heavy chain variable region may have an amino acid sequence selected from SEQ ID NOs: 23-27; and the light chain variable region may have an amino acid sequence of SEQ ID NO: 28.

In the embodiment of paragraph [0008], the polypeptide may be selected from:
a polypeptide wherein the heavy chain variable region has an amino acid sequence of SEQ ID NO: 23 and the light chain variable region has an amino acid sequence of SEQ ID NO: 28;
a polypeptide wherein the heavy chain variable region has an amino acid sequence of SEQ ID NO: 24 and the light chain variable region has an amino acid sequence of SEQ ID NO: 28;
a polypeptide wherein the heavy chain variable region has an amino acid sequence of SEQ ID NO: 25 and the light chain variable region has an amino acid sequence of SEQ ID NO: 28;
a polypeptide wherein the heavy chain variable region has an amino acid sequence of SEQ ID NO: 26 and the light chain variable region has an amino acid sequence of SEQ ID NO: 28;
a polypeptide wherein the heavy chain variable region has an amino acid sequence of SEQ ID NO: 27 and the light chain variable region has an amino acid sequence of SEQ ID NO: 28;
a polypeptide wherein the heavy chain variable region has an amino acid sequence of SEQ ID NO: 22 and the light chain variable region has an amino acid sequence of SEQ ID NO: 29; and
a polypeptide wherein the heavy chain variable region has an amino acid sequence of SEQ ID NO: 22 and the light chain variable region has an amino acid sequence of SEQ ID NO: 30.

In another aspect, the disclosure provides a conditionally active protein comprising a polypeptide of any of the embodiments of paragraphs [0008]-[0019].

In the embodiment of paragraph [0020], the conditionally active protein may bind to ApoE4.

In any one of the embodiments of paragraphs [0020]-[0021], the conditionally active protein may bind to ApoE with increased binding activity at an aberrant condition in comparison to the binding activity of the conditionally active protein at a normal physiological condition, and the conditionally active protein may bind to ApoE with a decreased binding activity at a normal physiological pH in comparison to a conditionally active protein having a heavy chain variable region of SEQ ID NO: 22 and a light chain variable region of SEQ ID NO: 28.

In any one of the embodiments of paragraphs [0020]-[0022], the conditionally active protein may be evolved from a parent protein and the binding activity of the conditionally active protein to ApoE at the normal physiological condition may be less than the binding activity of the parent protein at ApoE at the normal physiological condition.

In any one of the embodiments of paragraphs [0022]-[0023] above, the aberrant condition may be a pH in a range of from about 5.0 to about 7.0, or from about 5.4 to about 6.8, or from about 5.8 to about 6.8, or from about 6.0 to about 6.8, or from about 6.6 to about 6.8.

In any one of the embodiments of paragraphs [0022]-[0024], the normal physiological condition may be a pH is in a range of from about 7.2 to about 7.8, or from about 7.2 to about 7.6, or from about 7.2 to about 7.4.

In any one of the embodiments of paragraphs [0022]-[0025], the conditionally active protein may bind to ApoE at the aberrant condition with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

In any one of the embodiments of paragraphs [0020]-[0023], the conditionally active protein has a ratio of the binding activity to ApoE at the pH of the dementia brain to the binding activity to ApoE at the normal physiological pH of at least about 2:1, or at least about 5:1, or at least about 10:1, or at least about 20:1, or at least about 50:1, or at least about 100:1.

In the embodiment of paragraph [0027], the pH of the dementia brain may be in a range of from about 5.0 to about 7.0, or from about 5.4 to about 6.8, or from about 5.8 to about 6.8, or from about 6.0 to about 6.8, or from about 6.6 to about 6.8. and the normal physiological pH is in a range of from about 7.2 to about 7.8, or from about 7.2 to about 7.6, or from about 7.2 to about 7.4.

In any one of the embodiments of paragraphs [0027]-[0028], the conditionally active protein may bind to ApoE at the pH of the dementia brain with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

In any one of the embodiments of paragraphs [0020]-[0029], a therapeutically or prophylactically effective amount of the conditionally active protein may reduce ApoE4-amyloid R peptide binding by at least about 10%, at least about 20%, at least about 50%, at least about 90%, compared to the binding between ApoE4 and amyloid β peptide in the absence of the conditionally active protein.

In any one of the embodiments of paragraphs [0020]-[0030], a therapeutically or prophylactically effective amount of the conditionally active protein may reduce C-terminal cleavage of ApoE4 by at least about 5%, at least about 10%, at least about 20%, at least about 50%, at least about 90%, at least about 95%, at least about 99%, compared to the cleavage of ApoE4 in the absence of the conditionally active protein.

In any one of the embodiments of paragraphs [0020]-[0031], the conditionally active protein may bind to amyloid plaques.

In any one of the embodiments of paragraphs [0020]-[0032], the conditionally active protein may comprise at least one non-naturally occurring amino acid.

In any one of the embodiments of paragraphs [0020]-[0033], the conditionally active protein may be glycosylated.

In any one of the embodiments of paragraphs [0020]-[0034], the conditionally active protein may be an antibody or antigen binding antibody fragment.

In any one of the embodiments of paragraphs [0020]-[0034], the conditionally active protein may be a small peptide.

In any one of the embodiments of paragraphs [0020]-[0034], the conditionally active protein may be a cyclic peptide.

In the embodiment of paragraph [0035], the conditionally active protein may be a multi-specific antibody capable of binding to a receptor on the blood-brain barrier.

In the embodiment of paragraph [0038], the binding activity of the conditionally active protein to the receptor on the blood-brain barrier under a blood plasma physiological condition may be higher than the same binding activity to the blood-brain barrier (BBB) receptor under at least one brain physiological condition.

In any one of the embodiments of paragraphs [0038]-[0039], the receptor on the blood-brain barrier may be selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

In another aspect, the disclosure provides a conjugated conditionally active protein comprising the conditionally active protein of any one of the embodiments of paragraphs [0020]-[0040] conjugated to a ligand of a receptor on the blood-brain barrier, a polyamine, a therapeutic agent, a prophylactic agent, a diagnostic agent, a detectable label, a chelator or a contrast agent.

In the embodiment of paragraph [0041], the conditionally active protein may be conjugated to the ligand and the ligand is antibody of the receptor on the blood-brain barrier.

In the embodiment of paragraph [0041], the conditionally active protein may be conjugated to the ligand and the ligand is a natural ligand of the receptor on the blood-brain barrier or a modified ligand derived from a natural ligand of the receptor on the blood-brain barrier.

In the embodiment of paragraph [0041], the conditionally active protein may be conjugated to the ligand and the ligand is selected from a peptide having an amino acid sequence of SEQ ID NO: 18, 19, 20, or 21.

In the embodiment of paragraph [0041], the conditionally active protein may be conjugated to the ligand and the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

In the embodiment of paragraph [0041], the conditionally active protein may be conjugated to the polyamine.

In the embodiment of paragraph [0041], the conditionally active protein may be conjugated to the therapeutic agent or the prophylactic agent.

In the embodiment of paragraph [0041], the therapeutic or prophylactic agent may be selected from at least one of magnesium compounds, anti-excitotoxic compounds, growth factors, agents that bind to beta amyloid protein, calcium channel blockers, calcium chelators, potassium channel blockers, free radical scavengers, antioxidants, GABA agonists, GABA receptor antagonists, glutamate antagonists, NMDA antagonists, NMDA channel blockers, glycine site antagonists, polyamine site antagonists, adenosine receptor antagonists, leukocyte adhesion inhibitors, nitric oxide inhibitors, opioid antagonists, Serotonin agonists, sodium channel blockers, potassium channel openers, anti-inflammatory agents, and protein kinase inhibitors.

In the embodiment of paragraph [0048], the therapeutic or prophylactic agent may be the growth factor, and the growth factor may be selected from a Glial cell line derived neurotrophic factor, a brain derived neurotrophic factor, an insulin like growth factor, a fibroblast growth factor, and a neurotrophin.

In the embodiment of paragraph [0048], the therapeutic or prophylactic agent may be the calcium channel blocker, and the calcium channel blocker may be selected from nimodipine and flunarizine.

In the embodiment of paragraph [0041], the conditionally active protein may be conjugated to a diagnostic agent.

In yet another aspect, the disclosure provides a diagnostic agent comprising the conditionally active protein of any one of the embodiments of paragraphs [0020]-[0040], and a detectable label, a chelator or a contrast agent.

In the embodiment of paragraph [0052], the diagnostic agent may comprise the chelator and the chelator may be selected from at least one of ethylenediaminetetraacetic acid, [4-(1,4,8,11-tetraazacyclotetradec-1-yl) methyljbenzoic acid, cyclohexanediaminetetraacetic acid, ethylenebis(oxyethylenenitrilo)tetraacetic acid, diethylenetriaminepentaacetic acid, citric acid, hydroxyethyl ethylenediamine triacetic acid, iminodiacetic acid, triethylene tetraamine hexaacetic acid, 1,4,7, 10-tetraazacyclododecane-1,4,7, 10-tetra(methylene phosphonic acid), 1,4,8,11-tetraazacyclododecane-1,4,8, 11-tetraacetic acid, 1,4,7, 10-tetraazacyclododecane-1,4,7, 10-tetraacetic acid, and derivatives thereof.

In the embodiment of paragraph [0052], the diagnostic agent may comprise the detectable label and the detectable label may be selected from at least one of magnetic beads, fluorescent dyes, radiolabels, enzymes, and colorimetric labels.

In the embodiment of paragraph [0052], the diagnostic agent may comprise the contrast agent and the contrast agent may be selected from an x-ray contrast agent, gadolinium, dysprosium, and iron.

In another aspect, the disclosure provides a composition, kit or device comprising the conditionally active protein of any one of the embodiments of paragraphs [0020]-[0040], or the conjugated conditionally active protein of any one of the embodiments of paragraphs [0041]-[0051], or the diagnostic agent of any one of the embodiments of paragraphs [0052]-[0055].

In yet another aspect, the disclosure provides a method of generating, from a parent protein having a known binding activity to ApoE at a normal physiological pH, a conditionally active protein for prevention or treatment of a neurodegenerative disease, comprising steps of:
a) mutating the parent protein to generate a set of mutant proteins;
b) subjecting the set of mutant proteins to a first assay for binding activity to ApoE at a pH of a dementia brain and a second assay for binding activity to ApoE at a normal physiological pH; and
c) selecting the conditionally active protein from the mutant proteins of step b) that have an increased binding activity to ApoE in the first assay in comparison to the binding activity to ApoE in the second assay and which have a decreased binding activity to ApoE at a normal physiological pH in comparison to the parent protein.

In the embodiment of paragraph [0057], the pH in the dementia brain may be in a range of from about 5.0 to about 7.0, or from about 5.4 to about 6.8, or from about 5.6 to about 6.8, or from about 6.0 to about 6.8, or from about 6.4 to about 6.8 and the normal physiological pH is in a range of from about 7.2 to about 7.8, or from about 7.2 to about 7.6, or from about 7.2 to about 7.4.

In any one of the embodiments of paragraphs [0057]-[0058], assay solutions for the first and second assays may contain at least one component selected from at least one of:
(i) an inorganic compound,
(ii) an ion selected from a magnesium ion, a sulfate ion, a bisulfate ion, a carbonate ion, a bicarbonate ion, a nitrate ion, a nitrite ion, a phosphate ion, a hydrogen phosphate ion, a dihydrogen phosphate ion, a persulfate ion, a monopersulfate ion, a borate ion, an ammonium ion, a phosphorus ion, a sulfur ion, a chloride ion, a magnesium ion, a sodium ion, a potassium ion, an ammonium ion, an iron ion, a zinc ion, and a copper ion, and
(iii) an organic molecule other than polypeptides of SEQ ID NO. 4 and 5.

In the embodiment of paragraph [0059], the at least one component (i)-(iii) may have substantially the same concentration in the assay solutions for both the first and second assays.

In any one of the embodiments of paragraphs [0059]-[0060], the at least one component may comprise an inorganic compound selected from at least one of boric acid, calcium chloride, calcium nitrate, di-ammonium phosphate, magnesium sulfate, mono-ammonium phosphate, mono-potassium phosphate, potassium chloride, potassium sulfate, copper sulfate, iron sulfate, manganese sulfate, zinc sulfate, magnesium sulfate, calcium nitrate, calcium chelate, copper chelate, iron chelate, iron chelate, manganese chelate, zinc chelate, ammonium molybdate, ammonium sulphate, calcium carbonate, magnesium phosphate, potassium bicarbonate, potassium nitrate, hydrochloric acid, carbon dioxide, sulfuric acid, phosphoric acid, carbonic acid, uric acid, hydrogen chloride, and urea.

In any one of the embodiments of paragraphs [0059]-[0060], the at least one component may be selected from one or more of uric acid in concentration range of 2-7.0 mg/dL, calcium ion in a concentration range of 8.2-11.6 mg/dL, chloride ion in a concentration range of 355-381 mg/dL, iron ion in a concentration range of 0.028-0.210 mg/dL, potassium ion in a concentration range of 12.1-25.4 mg/dL, sodium ion in a concentration range of 300-330 mg/dL, and carbonic acid in a concentration range of 15-30 mM.

In any one the embodiments of paragraphs [0059]-[0060], the ion may be selected from at least one of magnesium ion, sulfate ion, bisulfate ion, carbonate ion, bicarbonate ion, nitrate ion, nitrite ion, phosphate ion, hydrogen phosphate ion, dihydrogen phosphate ion, persulfate ion, monopersulfate ion, borate ion, and ammonium ion.

### BRIEF DESCRIPTION OF THE DRAWINGS

FIG. 1 is a schematic representation of the structure of ApoE4.

FIG. 2 shows ApoE3 expression versus ApoE4 expression. Astrocytes expressing ApoE4 have inefficient lipid transport, sensitizing neurons to degeneration, and ApoE4 expression disrupts multiple homeostatic pathways in astrocytes and microglia to cause neurodegeneration and Alzheimer's disease.

FIGS. 3A-3D show pH affinity ELISA assays performed using amyloid beta peptides (FIG. 3A), low-density lipoprotein receptor (LDLR) (FIG. 3B), very low-density lipoprotein receptor (VLDR) (FIG. 3C), and sortilin (FIG. 3D) as coating antigens.

FIG. 4A shows the amino acid sequences for BAP191-VH-WT, BAP191-VH-M034D, BAP191-VH-1105D, BAP191-VH-R098E, BAP191-VH-M099E, and BAP191-VH-M104E.

FIG. 4B shows the amino acid sequences for BAP191-VK-WT, BAP191-VK-Y091D, and BAP191-VK-P095D.

FIGS. 5A-5B show anti-ApoE pH affinity ELISA with hApoE3 antigen at pH 6.0 (FIG. 5A) and at pH 7.4 (FIG. 5B).

FIGS. 6A-6B show anti-ApoE pH affinity ELISA with hApoE4 antigen at pH 6.0 (FIG. 6A) and at pH 7.4 (FIG. 6B).

FIGS. 7A-7B show anti-ApoE pH affinity ELISA with mouse ApoE antigen at pH 6.0 (FIG. 7A) and at pH 7.4 (FIG. 7B).

### DEFINITIONS

In order to facilitate understanding of the examples provided herein, certain frequently occurring methods and/or terms will be defined herein.

The definitions of the terms “about,” “activity,” “agent,” “ambiguous base requirement,” “amino acid,” “amplification,” “chimeric property,” “cognate,” “comparison window,” “conservative amino acid substitutions,” “corresponds to,” “degrading effective,” “defined sequence framework,” “digestion,” “directional ligation,” “DNA shuffling,” “drug” or “drug molecule,” “effective amount,” “electrolyte,” “epitope,” “enzyme,” “evolution” or “evolving,” “fragment,” “derivative,” “analog,” “full range of single amino acid substitutions,” “gene,” “genetic instability,” “heterologous,” “homologous” or “homologous,” “industrial applications,” “identical” or “identity,” “areas of identity,” “isolated,” “isolated nucleic acid,” “ligand,” “ligation,” “linker” or “spacer,” “microenvironment,” “molecular property to be evolved,” “mutations,” “naturally-occurring,” “normal physiological conditions” or “wild type operating conditions,” “nucleic acid molecule,” “nucleic acid molecule,” “nucleic acid sequence coding for” or “DNA coding sequence of” or a “nucleotide sequence encoding,” “promotor sequence,” “nucleic acid encoding an enzyme (protein)” or “DNA encoding an enzyme (protein)” or “polynucleotide encoding an enzyme (protein),” “specific nucleic acid molecule species,” “assembling a working nucleic acid sample into a nucleic acid library,” “nucleic acid library,” “nucleic acid construct” or “nucleotide construct” or “DNA construct,” “construct,” “oligonucleotide” or “oligo,” “homologous,” “operably linked,” “operably linked to,” “parental polynucleotide set,” “patient” or “subject,” “physiological conditions,” “population,” “pro-form,” “pre-pro-form,” “pseudorandom,” “quasi-repeated units,” “random peptide library,” “random peptide sequence,” “receptor,” “recombinant,” “synthetic,” “related polynucleotides,” “reductive reassortment,” “reference sequence,” “comparison window,” “sequence identity,” “percentage of sequence identity,” “substantial identity,” “reference sequence,” “repetitive index (RI)”, “restriction site,” “selectable polynucleotide,” “sequence identity,” “similarity,” “specifically bind,” “specific hybridization,” “specific polynucleotide,” “stringent hybridization conditions,” “substantially identical,” “substantially pure enzyme,” “substantially pure,” “treating,” “variable segment,” “variant,” “working,” “conditionally active antibody,” “antibody-dependent cell-mediated cytotoxicity” or “ADCC,” “cancer” and “cancerous,” “multispecific antibody,” “full length antibody,” “library,” “recombinant antibody,” and “individual” or “subject” are the same as in WO 2016/138071 and thus are hereby incorporated by reference in their entirety herein.

As used herein, the phrase “amyloid plaques” refers to insoluble fibrous amyloid β peptide aggregates having a beta-pleated sheet structure and that stains with Congo Red dye. Plaques may also be referred to as deposits. These misfolded structures alter the normal configuration amyloid R peptide such that they undesirably interact with one another or other cell components to form insoluble fibrils (plaques). Abnormal accumulation of amyloid fibrils in organs may lead to amyloidosis and may play a role in development of various neurodegenerative diseases.

The term “antibody” as used herein refers to intact immunoglobulin molecules, as well as fragments of immunoglobulin molecules, such as Fab, Fab′, (Fab′)₂, Fv, and single chain antibody (SCA or scFv) fragments, that are capable of binding to an epitope of an antigen. These antibody fragments, which retain some ability to selectively bind to an antigen (e.g., a polypeptide antigen) of the antibody from which they are derived, can be made using well known methods in the art (see, e.g., Harlow and Lane, supra), and are described further, as follows. Antibodies useful in the practice of the claimed invention may be IgG1, IgG2, IgG3, IgG4, IgM, IgA1, IgA2, sIgA, IgD or IgE. Antibodies can be used to isolate preparative quantities of the antigen by immunoaffinity chromatography. Various other uses of such antibodies are to diagnose and/or stage disease (e.g., neoplasia) and for therapeutic application to treat disease, such as for example: neoplasia, autoimmune disease, AIDS, cardiovascular disease, infections, and the like. Chimeric, human-like, humanized or fully human antibodies are particularly useful for administration to human patients.

An Fab fragment consists of a monovalent antigen-binding fragment of an antibody molecule, and can be produced by digestion of a whole antibody molecule with the enzyme papain, to yield a fragment consisting of an intact light chain and a portion of a heavy chain.

An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

An (Fab′)2 fragment of an antibody can be obtained by treating a whole antibody molecule with the enzyme pepsin, without subsequent reduction. A (Fab′)₂ fragment is a dimer of two Fab′ fragments, held together by two disulfide bonds.

An Fv fragment is defined as a genetically engineered fragment containing the variable region of a light chain and the variable region of a heavy chain expressed as two chains.

A single chain antibody (“SCA” or scFv) is a genetically engineered single chain molecule containing the variable region of a light chain and the variable region of a heavy chain, linked by a suitable, flexible polypeptide liner, and which may include additional amino acid sequences at the amino- and/or carboxyl-termini. For example, a single chain antibody may include a tether segment for linking to the encoding polynucleotide. A functional single chain antibody generally contains a sufficient portion of the variable region of a light chain and a sufficient region of the variable region of a heavy chain so as to retain the property of a full-length antibody for binding to a specific target molecule or epitope.

The term “ApoE cleavage enzyme” (“AECE”) as used herein is an enzyme that cleaves ApoE, e.g., ApoE4, to yield neurotoxic ApoE fragments. An AECE is a serine protease. In some embodiments, an AECE is present in a mature neuron at higher levels than in an immature neuron. For example, an AECE may be present in a mature neuron at a level that is about 25%, about 50%, about 2-fold, about 5-fold, about 10-fold, or more than 10-fold higher than the AECE level in an immature neuron. In some embodiments, an AECE may be present in cortical and hippocampal neurons at higher levels than in cerebellar neurons. For example, an AECE may be present in cortical and hippocampal neurons at a level that is about 25%, about 50%, about 2-fold, about 5-fold, about 10-fold, or more than 10-fold higher than the AECE level in cerebellar neurons. In some embodiments, an AECE may be present in neurons at much higher levels than in astrocytes. For example, an AECE may be present in mature neurons at a level that is about 2-fold, about 5-fold, about 10-fold, about 25-fold, about 50-fold, or about 100-fold, or greater than 100-fold, higher than the AECE level in an astrocyte.

The term “ApoE4” as used herein refers to the ApoE4 allele of the ApoE gene or ApoE4 protein. The term is used interchangeably with the ApoE4 gene and ApoE4 protein.

The term “brain trauma” as used herein refers to an acquired brain injury or a head injury, when a trauma causes damage to the brain. Trauma includes, e.g., post-head trauma, impact trauma, and other traumas to the head such as traumas caused by accidents and/or sports injuries, concussive injuries, penetrating head wounds, brain tumors, stroke, heart attack, meningitis, viral encephalitis, and other conditions that deprive the brain of oxygen. In a particular embodiment, the brain trauma may be caused by an external, physical force.

The term “conditionally active protein” refers to a variant, or mutant, of a parent protein which is more or less active at one or more aberrant conditions as compared to the activity of the conditionally active protein at a control or normal physiological condition. The conditionally active protein may also be more active at one or more aberrant conditions as compared to the activity of the conditionally active protein at a control or normal condition, and the activity of the conditionally active protein at the control or normal physiological condition is less than the activity of the parent protein at the control or normal condition. A conditionally active protein exhibits activity in selected regions of the body and/or exhibits increased or decreased activity under aberrant, or permissive, physiological conditions. Normal physiological conditions are those which would be considered to be within a normal range at a location in a subject such as at the site of administration, or at the tissue or organ at the site of action, in a subject. An aberrant condition is that which deviates from the normally acceptable range for that condition at that location. In one aspect, the conditionally active protein is virtually inactive at a normal physiological condition but is active at the aberrant or permissive condition. For example, in one aspect, an evolved conditionally active protein is virtually inactive at a normal physiological pH, but is active at lower pH in the dementia brain. In another aspect, the conditionally active protein may be reversibly or irreversibly inactivated at a normal physiological pH. In a yet another aspect, the evolved conditionally active protein has greater activity at the aberrant or permissive condition than at the normal physiological condition, and the activity of the evolved conditionally active protein at the normal physiological condition is less than the activity of the parent protein at the normal physiological condition. For example, the evolved conditionally active protein has greater activity at a lower pH in the dementia brain than at a normal physiological pH of blood, but the activity of the evolved conditionally active protein at the normal physiological pH of blood is less than the activity of the parent protein at the normal physiological pH of blood. In a further aspect, the conditionally active protein is a therapeutic or prophylactic protein. In another aspect, the conditionally active protein is used as a drug, or therapeutic or prophylactic agent. A conditionally active protein may be a conditionally active biologic protein. A conditionally active protein may also be a conditionally active antibody.

As used herein, the term “cyclic peptide” refers to a polypeptide chain that forms a circular chain, for example, wherein the amino and carboxyl termini are linked together with a peptide bond that forms the circular chain (e.g., between the alpha carboxyl of one residue and the alpha amine of another). For purposes of this patent application, cyclic peptides may also include linkage other than a peptide bond such as non-alpha amide linkage, e.g. a thioether linkage between Trp and Cys residues. The length of the cyclic peptide may be in the range of from about 5 to about 500 amino acids, or from about 8 to about 300 amino acids, or from about 8 to about 200 amino acids, or from about 10 to about 50 or 100 amino acids. Additionally, amino acids other than naturally-occurring amino acids, for example β-alanine, phenyl glycine and homoarginine, may be included in the cyclic peptides.

The term “dementia brain” as used herein refers to a brain, a portion of the brain or a portion of the spinal cord of a person or animal having a neurodegenerative disease where progressive loss of neuronal function and structure, or neuron death occurs. Typically, the dementia brain exhibits pathological changes associated with loss of cognitive functions in the person or animal.

The term “epitope” or “antigenic determinant” as used herein refers to a site on an antigen to which an antibody binds. Epitopes can be formed both from contiguous amino acids (linear epitope) or noncontiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope can comprise 3 or more amino acids. Usually an epitope consists of at least 5 to 7 amino acids (such as 5, 6, or 7 amino acids in an epitope), or of at least 8-11 amino acids (such as 8, 9, 10 or 11 amino acids in an epitope), or of more than 11 amino acids (such as 12, 13, 14, 15, 16, 17, 18, 19 or 20 amino acid in an epitope), or of more than 20 amino acids (such as 21, 22, 23, 24, 25, 26, 27, 28, 29, or 30 amino acid in an epitope), less frequently even of 31-40 amino acids. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). A preferred method for epitope mapping on an antigen is surface plasmon resonance.

The term “full length antibody” refers to an antibody which comprises an antigen-binding variable region (V_{H} or V_{L}) as well as a light chain constant domain (CL) and heavy chain constant domains, CH1, CH2 and CH3. The constant domains may be native sequence constant domains (e.g. human native sequence constant domains) or amino acid sequence variants thereof. Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively.

An “individual,” “patient” or “subject” is a human or an animal. For example, the subject is a mammal selected from domesticated animals (e.g., cows, sheep, cats, dogs, and horses), primates (e.g., humans and non-human primates such as monkeys), rabbits, and rodents (e.g., mice and rats).

The term “library” as used herein refers to a collection of nucleic acids or proteins in a single pool. The library may be generated using DNA recombinant technology. For example, a collection of cDNAs or any other protein coding DNAs may be inserted in an expression vector to generate a protein library. A collection of cDNAs or protein coding DNAs may also be inserted into a phage genome to generate a bacteriophage display library of wild-type proteins. The collection of cDNAs may be produced from a selected cell population or a tissue sample, such as by the methods disclosed by Sambrook et al. (Molecular Cloning, Cold Spring Harbor Laboratory Press, 1989). cDNA collections from selected cell types are also commercially available from vendors such as Stratagene®. The library of wild-type proteins as used herein is not a collection of biological samples.

The term “multispecific antibody” as used herein is a full-length antibody or antibody fragment having binding specificities for at least two different epitopes on the same antigen or different antigen. Exemplary multispecific antibodies may bind both a BBB receptor and ApoE4. Multispecific antibodies can be prepared as full-length antibodies or antibody fragments (e.g. F(ab′)₂ bispecific antibodies). Engineered antibodies with two, three or more (e.g. four) functional antigen binding sites are also contemplated (see, e.g., US 2002/0004587 A1).

The terms “neurodegenerative condition” or “neurodegenerative disease” are used interchangeably in this application, to refer to a neurological condition or disease characterized by a progressive loss of neuronal function and structure, and/or neuron death. Clinically, neurodegenerative diseases are characterized by a slow onset and chronic progression of motion impairment and memory loss. In a neurodegenerative disease, a particular part of the brain, spinal cord, or peripheral nerve functionally fails and the neurons of the dysfunctional region die. Neurodegenerative diseases are often categorized by whether they initially affect cognition, movement, strength, coordination, sensation, or autonomic control. However, it is not uncommon for patients to be presented with symptoms and signs affecting more than one system. While it is possible that involvement of several systems can occur concomitantly, typically by the time the patient's function has declined sufficiently to seek medical attention multiple systems are involved. Non-limiting examples of neurodegenerative diseases or conditions include Parkinson's disease (PD), Alzheimer's disease (AD), Huntington's disease (HD), age-related macular degeneration (AMD), retinitis pigmentosa (RP), amyotrophic lateral sclerosis (ALS, e.g., familial ALS and sporadic ALS), multiple system atrophy, progressive supranuclear palsy, down syndrome, diffuse Lewy body disease, multiple sclerosis (MS), and brain trauma.

The term “parent protein” as used herein refers to a polypeptide or protein that may be evolved to produce a conditionally active protein using the methods of the present invention. The parent protein may be a wild-type protein or a non-naturally occurring protein. For example, a therapeutic or prophylactic polypeptide or protein or a mutant or variant of a polypeptide or protein may be used as a parent polypeptide or protein. Parent protein may also be a fragment of another naturally occurring protein, wild-type protein, therapeutic or prophylactic protein or mutant protein. Examples of parent proteins include antibodies, antibody fragments, enzymes, enzyme fragments, cytokines and fragments thereof, hormones and fragments thereof, ligands and fragments thereof, receptors and fragments thereof, regulatory proteins and fragments thereof, and growth factors and fragments thereof.

The term “pharmaceutically acceptable salt” as used herein refers to a salt form of the conditionally active protein of the present invention. The salt form may be acid addition salts (e.g., formed with free amino groups) and which are formed with inorganic acids such as hydrochloric or phosphoric acids, or such organic acids such as acetic, oxalic, tartaric and maleic. Salts formed with the free carboxyl groups may also be derived from inorganic bases such as sodium, potassium, ammonium, calcium, or ferric hydroxides, and organic bases as isopropylamine, trimethylamine, 2-ethylamino ethanol, histidine and procaine.

The term “polypeptide” as used herein refers to a polymer in which the monomers are amino acids and are joined together through peptide bonds. A polypeptide may be a full-length naturally-occurring amino acid chain or a fragment, mutant or variant thereof, such as a selected region of the amino acid chain that is of interest in a binding interaction. A polypeptide may also be a synthetic amino acid chain, or a combination of a naturally-occurring amino acid chain or fragment thereof and a synthetic amino acid chain. A fragment refers to an amino acid sequence that is a portion of a full-length protein, and will be typically between about 8 and about 500 amino acids in length, about 8 to about 300 amino acids, about 8 to about 200 amino acids, and about 10 to about 50 or 100 amino acids in length. Additionally, amino acids other than naturally-occurring amino acids, for example β-alanine, phenyl glycine and homoarginine, may be included in the polypeptides. Commonly-encountered amino acids which are not gene-encoded may also be included in the polypeptides. The amino acids may be either the D- or L-optical isomer. In addition, other peptidomimetics are also useful, e.g. in linker sequences of polypeptides (see Spatola, 1983, in Chemistry and Biochemistry of Amino Acids, Peptides and Proteins, Weinstein, ed., Marcel Dekker, New York, p. 267). In general, the term “protein” is not intended to convey any significant difference from the term “polypeptide” other than to include structures which may comprise two or several polypeptide chains held together by covalent or non-covalent bonds.

The term “preventing” as used herein refers to avert or avoid a condition from occurring. In some embodiments, preventing is directed to ameliorating the damage associated with a condition, such as a neurodegenerative disease.

The term “small molecule” as used herein refers to molecules or ions that have a molecular weight of less than 900 a.m.u., or less than 500 a.m.u. or less than 200 a.m.u. or less than 100 a.m.u. In the assays and environments of the present invention, small molecules may often be present as a mixture of the molecule and a deprotonated ion of the molecule, depending primarily on the pH of the assay or environment.

The term “small peptide” as used herein is referred to a peptide consisting of at most 25, 24, 23, 22, 21, 20, 19, 18, 17, 16, 15, 14, 13, 12, 11, 10, 9, 8, 7, 6, 5 amino acid residues. The small peptide may be a linear chain of amino acid residues or a branched chain of amino acid residues. In some embodiments, the small peptide may be a cyclic peptide.

The term “therapeutically effective amount” as used herein means any amount which, as compared to a corresponding subject who has not received such amount, results in, but is not limited to, healing, prevention, or amelioration of a disease, disorder, or side effect, or a decrease in the rate of advancement of a disease or disorder. The term also includes within its scope amounts effective to enhance normal physiological function as well as amounts effective to cause a physiological function in a patient which enhances or aids in the therapeutic effect of a second pharmaceutical agent.

The term “prophylactically effective amount” as used herein means any amount which, as compared to a corresponding subject who has not received such amount, results in, but is not limited to prevention, or amelioration of a disease, disorder, or side effect, or a decrease in the rate of advancement of a disease or disorder. The term also includes within its scope amounts effective to enhance normal physiological function as well as amounts effective to cause a physiological function in a patient which enhances or aids in the therapeutic or prophylactic effect of a second pharmaceutical agent.

The term “treating” or “treatment” includes reducing the number of symptoms or reducing the severity, duration, or frequency of one or more symptoms of disease (e.g., a neurodegenerative disease) in a subject. The term treating can also mean delaying the onset or progression of symptoms, or progression of severity of symptoms, of a neurodegenerative disorder in a subject, or increasing the longevity of a subject having a neurodegenerative disorder. Some exemplary symptoms include. but are not limited to: accumulation, oligomerization, and deposition of amyloid beta (A); tau phosphorylation; intraneuronal, lysosomal, and mitochondrial pathology in at least one region of the brain; lysosomal, an autophagy impairments and synaptic and neuronal loss;
Behavioral dysfunction, such as movement impairments and loss of memory; and the like.

The term “unit dosage form” as used herein refers to physically discrete units suitable as unitary dosages for a subject, each unit containing a predetermined quantity of conditionally active protein of the present invention calculated in an amount sufficient to produce the desired therapeutic or prophylactic effect in association with a pharmaceutically acceptable diluent, carrier or vehicle.

### DETAILED DESCRIPTION

It must be noted that as used herein and in the appended claims, the singular forms “a”, “an”, and “the” include plural references unless the context clearly dictates otherwise. Furthermore, the terms “a” (or “an”), “one or more,” and “at least one” can be used interchangeably herein. The terms “comprising,” “including,” “having,” and “constructed from” can also be used interchangeably.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It is to be understood that each component, compound, substituent or parameter disclosed herein is to be interpreted as being disclosed for use alone or in combination with one or more of each and every other component, compound, substituent or parameter disclosed herein.

It is also to be understood that each amount/value or range of amounts/values for each component, compound, substituent or parameter disclosed herein is to be interpreted as also being disclosed in combination with each amount/value or range of amounts/values disclosed for any other component(s), compounds(s), substituent(s) or parameter(s) disclosed herein and that any combination of amounts/values or ranges of amounts/values for two or more component(s), compounds(s), substituent(s) or parameters disclosed herein are thus also disclosed in combination with each other for the purposes of this description.

It is further understood that each range disclosed herein is to be interpreted as a disclosure of each specific value within the disclosed range that has the same number of significant digits. Thus, a range of from 1-4 is to be interpreted as an express disclosure of the values 1, 2, 3 and 4. It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

### Isolated Polypeptides

The present invention provides an isolated polypeptide comprising a heavy chain variable region having three complementarity determining regions having H1, H2, and H3 sequences, wherein:
(a) the H1 sequence is GYTFTTAGX₁Q (SEQ ID NO: 31),
(b) the H2 sequence is WX₂NTHSGEPKYAEDFKG (SEQ ID NO: 32), and
(c) the H3 sequence is X₃GGYAX₄DY (SEQ ID NO: 33);
wherein X₁ is M or D,
   X₂ is I or D,
   X₃ is M or E, and
   X₄ is M or E; and
   a light chain variable region having three complementarity determining regions having L1, L2, and L3 sequences, wherein:
(a) the L1 sequence is KASEDINSYLS (SEQ ID NO: 34),
(b) the L2 sequence is RANRLVD (SEQ ID NO: 35), and
(c) the L3 sequence is LQX₅DEFX₆LT (SEQ ID NO: 36);
wherein X₅ is Y or D and X₆ is P or D;
with the proviso that X₁, X₂, X₃, X₄, X₅ and X₆ cannot be M, I, M, M, Y, P, respectively, at the same time.

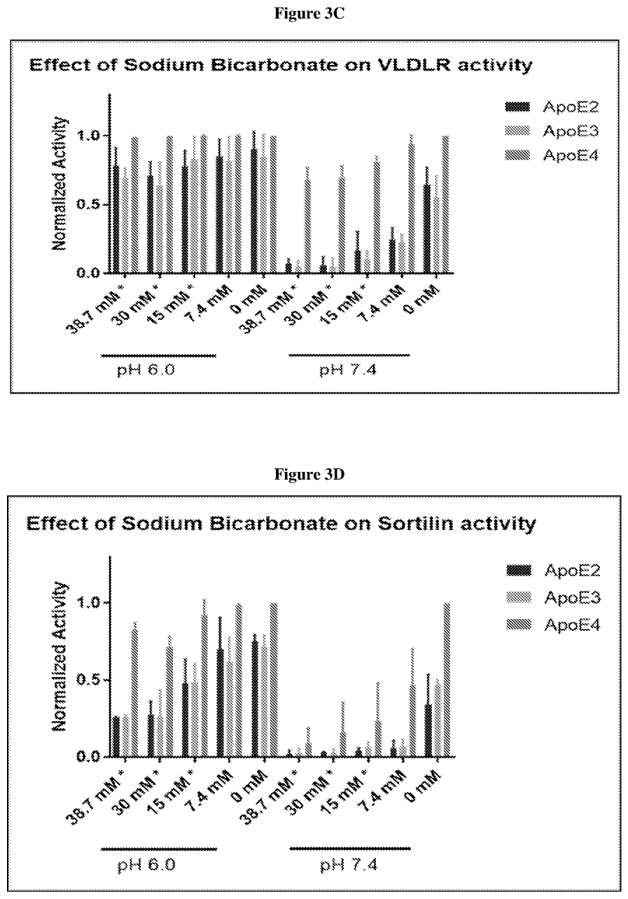

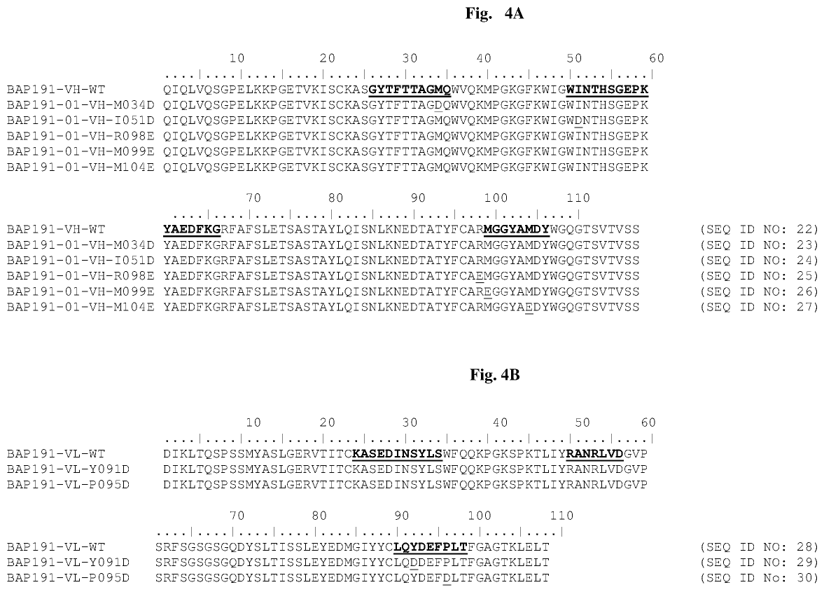

The alignment of the heavy chain variable regions is shown in FIG. 4A. The alignment of the light chain variable regions is shown in FIG. 4B.

In one embodiment of the isolated polypeptide, the heavy chain variable region comprises an H1 sequence selected from SEQ ID NOs: 37 and 38, an H2 sequence selected from SEQ ID NOs: 39 and 40, and an H3 sequence selected from SEQ ID NOs: 44-46.

In any one of the embodiments of the isolated polypeptide, the heavy chain variable region is selected from:
the heavy chain variable region having the H1 sequence of SEQ ID NO: 37, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 40, and the H3 sequence of SEQ ID NO: 44;
the heavy chain variable region having the H1 sequence of SEQ ID NO: 38, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 39, and the H3 sequence of SEQ ID NO: 45; and
the heavy chain variable region having the H1 sequence of SEQ ID NO: 37, the H2 sequence of SEQ ID NO: 39, and the H3 sequence of SEQ ID NO: 46.

In a second embodiment of the isolated polypeptide, the heavy chain variable region of the isolated polypeptide has an amino acid sequence selected from the amino acid sequences of SEQ ID NOs: 22-27.

In any one of the embodiments of the isolated polypeptide, the light chain variable region has an amino acid sequence selected from the amino acid sequences of SEQ ID NOs: 28-30.

In a third embodiment of the isolated polypeptide, the L1 sequence is SEQ NO: 34, the L2 sequence is SEQ ID NO: 35, and the L3 sequence is selected from SEQ ID NOs: 41-43.

In a fourth embodiment of the isolated polypeptide, the L1 sequence is SEQ NO: 34, the L2 sequence is SEQ ID NO: 35, and the L3 sequence is selected from SEQ ID NOs: 41-43, and the heavy chain variable region comprises the H1 sequence selected from SEQ ID NOs: 37 and 38, the H2 sequence selected from SEQ ID NOs: 39 and 40, and the H3 sequence selected from SEQ ID NOs: 44-46.

In a fifth embodiment of the isolated polypeptide, the L1 sequence is SEQ NO: 34, the L2 sequence is SEQ ID NO: 35, and the L3 sequence is selected from SEQ ID NOs: 42-43, and the heavy chain variable region is selected from:
the heavy chain variable region having the H1 sequence of SEQ ID NO: 37, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 40, and the H3 sequence of SEQ ID NO: 44;
the heavy chain variable region having the H1 sequence of SEQ ID NO: 38, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 39, and the H3 sequence of SEQ ID NO: 45; and
the heavy chain variable region having the H1 sequence of SEQ ID NO: 37, the H2 sequence of SEQ ID NO: 39, and the H3 sequence of SEQ ID NO: 46.

In a sixth embodiment of the isolated polypeptide, the L1 sequence is SEQ ID NO: 34, the L2 sequence is SEQ ID NO: 35, and the L3 sequence is SEQ ID NO: 41; and
the heavy chain variable region is selected from:
the heavy chain variable region having the H1 sequence of SEQ ID NO: 37, the H2 sequence of SEQ ID NO: 40, and the H3 sequence of SEQ ID NO: 44;
the heavy chain variable region having the H1 sequence of SEQ ID NO: 38, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 39, and the H3 sequence of SEQ ID NO: 45; and
the heavy chain variable region having the H1 sequence of SEQ ID NO: 37, the H2 sequence of SEQ ID NO: 39, and the H3 sequence of SEQ ID NO: 46.

In a seventh embodiment of the isolated polypeptide, the heavy chain variable region has the H1 sequence of SEQ ID NO: 37, the H2 sequence of SEQ ID NO: 39, and the H3 sequence of SEQ ID NO: 44; and the light chain variable region has the L1 sequence of SEQ ID NO: 34, the L2 sequence of SEQ ID NO: 35, and the L3 sequence selected from SEQ ID NOs: 42-43.

In an eighth embodiment of isolated polypeptide, the heavy chain variable region has the amino acid sequence of SEQ ID NO: 22; and the light chain variable region has an amino acid sequence selected from the amino acid sequences of SEQ ID NOs: 29-30.

In a ninth embodiment of the isolated polypeptide, the heavy chain variable region has an amino acid sequence selected from SEQ ID NOs: 23-27; and the light chain variable region has the amino acid sequence of SEQ ID NO: 28.

In the embodiment, the polypeptide is selected from:
a polypeptide wherein the heavy chain variable region has an amino acid sequence of SEQ ID NO: 23 and the light chain variable region has an amino acid sequence of SEQ ID NO: 28;
a polypeptide wherein the heavy chain variable region has an amino acid sequence of SEQ ID NO: 24 and the light chain variable region has an amino acid sequence of SEQ ID NO: 28;
a polypeptide wherein the heavy chain variable region has an amino acid sequence of SEQ ID NO: 25 and the light chain variable region has an amino acid sequence of SEQ ID NO: 28;
a polypeptide wherein the heavy chain variable region has an amino acid sequence of SEQ ID NO: 26 and the light chain variable region has an amino acid sequence of SEQ ID NO: 28;
a polypeptide wherein the heavy chain variable region has an amino acid sequence of SEQ ID NO: 27 and the light chain variable region has an amino acid sequence of SEQ ID NO: 28;
a polypeptide wherein the heavy chain variable region has an amino acid sequence of SEQ ID NO: 22 and the light chain variable region has an amino acid sequence of SEQ ID NO: 29; and
a polypeptide wherein the heavy chain variable region has an amino acid sequence of SEQ ID NO: 22 and the light chain variable region has an amino acid sequence of SEQ ID NO: 30.

In a tenth embodiment of the isolated polypeptide, the isolated polypeptide is useful for prevention or treatment of a neurodegenerative disease.

In any one of the embodiments of the isolated polypeptide, the isolated polypeptide binds to ApoE.

In any one of the embodiments of the isolated polypeptide, the isolated polypeptide binds to ApoE4.

In any one of the embodiments of the isolated polypeptide, the isolated polypeptide binds to ApoE3 and/or ApoE4.

In any one of the embodiments of the isolated polypeptide, the isolated polypeptide binds to ApoE with increased binding activity at an aberrant condition in comparison to the binding activity of the isolated polypeptide to ApoE at a normal physiological condition.

In any one of the embodiments of the isolated polypeptide, the isolated polypeptide binds to ApoE4 with increased binding activity at an aberrant condition in comparison to the binding activity of the isolated polypeptide to ApoE4 at a normal physiological condition.

In any one of the embodiments of the isolated polypeptide, the isolated polypeptide binds to ApoE with increased binding activity at an aberrant condition in comparison to the binding activity of the isolated polypeptide to ApoE at a normal physiological condition, and the isolated polypeptide binds to ApoE with a decreased binding activity at the normal physiological condition in comparison to the binding activity of an isolated polypeptide having a heavy chain variable region of SEQ ID NO: 22 and a light chain variable region of SEQ ID NO: 28.

In any one of the embodiments of the isolated polypeptide, the isolated polypeptide is evolved from a parent polypeptide and the binding activity of the isolated polypeptide to ApoE at the normal physiological condition is less than the binding activity of the parent polypeptide to ApoE at the normal physiological condition.

In any one of the embodiments of the isolated polypeptide, the isolated polypeptide binds to ApoE with an increased binding activity at an aberrant pH in a range of from about 5.0 to about 7.0, or from about 5.4 to about 6.8, or from about 5.8 to about 6.8, or from about 6.0 to about 6.8, or from about 6.6 to about 6.8 in comparison to the binding activity of the isolated polypeptide to ApoE at a normal physiological pH, and the isolated polypeptide binds to ApoE with a decreased binding activity at a normal physiological pH in comparison to the binding activity to ApoE of an isolated polypeptide having a heavy chain variable region of SEQ ID NO: 22 and a light chain variable region of SEQ ID NO: 28.

In any one of the embodiments of the isolated polypeptide, the isolated polypeptide binds to ApoE4 with an increased binding activity at an aberrant pH in a range of from about 5.0 to about 7.0, or from about 5.4 to about 6.8, or from about 5.8 to about 6.8, or from about 6.0 to about 6.8, or from about 6.6 to about 6.8, in comparison to the binding activity of the isolated polypeptide to ApoE4 at a normal physiological pH and wherein the isolated polypeptide binds to ApoE4 with a decreased binding activity at a normal physiological pH in comparison to the binding activity to ApoE4 of an isolated polypeptide having a heavy chain variable region of SEQ ID NO: 22 and a light chain variable region of SEQ ID NO: 28.

In any one of the embodiments of the isolated polypeptide, the normal physiological condition is a pH is in a range of from about 7.2 to about 7.8, or from about 7.2 to about 7.6, or from about 7.2 to about 7.4.

In any one of the embodiments of the isolated polypeptide, the isolated polypeptide binds to ApoE at an aberrant condition with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

In any one of the embodiments of the isolated polypeptide in which the isolated polypeptide is useful for prevention or treatment of a neurodegenerative disease, the isolated polypeptide binds to ApoE with an increased binding activity at a pH of a dementia brain in comparison with the binding activity to ApoE at a normal physiological pH.

In any one of the embodiments of the isolated polypeptide in which the isolated polypeptide is useful for prevention or treatment of a neurodegenerative disease, the isolated polypeptide has a ratio of binding activity to ApoE at a pH of a dementia brain to the binding activity of the isolated polypeptide to ApoE at a normal physiological pH of at least about 2:1, or at least about 5:1, or at least about 10:1, or at least about 20:1, or at least about 50:1, or at least about 100:1.

In the above embodiment, the pH of the dementia brain is in a range of from about 5.0 to about 7.0, or from about 5.4 to about 6.8, or from about 5.8 to about 6.8, or from about 6.0 to about 6.8, or from about 6.6 to about 6.8. and the normal physiological pH is in a range of from about 7.2 to about 7.8, or from about 7.2 to about 7.6, or from about 7.2 to about 7.4.

In the above embodiment, the isolated polypeptide binds to ApoE at the pH of the dementia brain with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

In any one of the embodiments, a therapeutically or prophylactically effective amount of the isolated polypeptide reduces ApoE4-amyloid β peptide binding by at least about 10%, at least about 20%, at least about 50%, at least about 90%, compared to the binding between ApoE4 and amyloid β peptide in the absence of the isolated polypeptide.

In one of the embodiments, a therapeutically or prophylactically effective amount of the isolated polypeptide reduces C-terminal cleavage of ApoE4 by at least about 5%, at least about 10%, at least about 20%, at least about 50%, at least about 90%, at least about 95%, at least about 99%, compared to the cleavage of ApoE4 in the absence of the isolated polypeptide.

In any one of the embodiments of the isolated polypeptide, the isolated polypeptide binds to amyloid plaques.

In any one of the embodiments of the isolated polypeptide, the isolated polypeptide comprises at least one non-naturally occurring amino acid.

In any one of the embodiments of the isolated polypeptide, the isolated polypeptide is glycosylated.

In any one of the embodiments of the isolated polypeptide, the isolated polypeptide is a small peptide.

In any one of the embodiments of the isolated polypeptide, the isolated polypeptide is a cyclic peptide.

In any one of the embodiments of the isolated polypeptide, the isolated polypeptide is an antibody or antigen binding antibody fragment.

In the embodiment in which the isolated polypeptide is an antibody or antigen binding antibody fragment, the isolated polypeptide is a multi-specific antibody capable of binding to a receptor on the blood-brain barrier.

In the embodiment in which the isolated polypeptide is a multi-specific antibody capable of binding to a receptor on the blood-brain barrier, the binding activity of the isolated polypeptide to the receptor on the blood-brain barrier under a blood plasma physiological condition is higher than the same binding activity to the BBB receptor under at least one brain physiological condition.

In the embodiment in which the isolated polypeptide is a multi-specific antibody capable of binding to a receptor on the blood-brain barrier, the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

In the embodiment in which the isolated polypeptide is a multi-specific antibody capable of binding to a receptor on the blood-brain barrier, the binding activity of the isolated polypeptide to the receptor on the blood-brain barrier under a blood plasma physiological condition is higher than the same binding activity to the BBB receptor under at least one brain physiological condition; and the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

In a further embodiment, the present invention provides a diagnostic agent comprising the isolated polypeptide of any one of the embodiments, and a detectable label, a chelator or a contrast agent.

In the above embodiment, the diagnostic agent comprises the chelator and the chelator is selected from at least one of ethylenediaminetetraacetic acid, [4-(1,4,8, 11-tetraazacyclotetradec-1-yl) methyljbenzoic acid, cyclohexanediaminetetraacetic acid, ethylenebis(oxyethylenenitrilo)tetraacetic acid, diethylenetriaminepentaacetic acid, citric acid, hydroxyethyl ethylenediamine triacetic acid, iminodiacetic acid, triethylene tetraamine hexaacetic acid, 1,4,7, 10-tetraazacyclododecane-1,4,7, 10-tetra(methylene phosphonic acid), 1,4,8,11-tetraazacyclododecane-1,4,8, 11-tetraacetic acid, 1,4,7, 10-tetraazacyclododecane-1,4,7, 10-tetraacetic acid, and chelating derivatives thereof.

In any one embodiment, the diagnostic agent comprises the detectable label and the detectable label is selected from at least one of magnetic beads, fluorescent dyes, radiolabels, enzymes, and colorimetric labels.

In any one embodiment, the diagnostic agent comprises the contrast agent and the contrast agent is selected from an x-ray contrast agent, gadolinium, dysprosium, and iron.

A further embodiment, the present invention provides a composition, kit or device comprising the isolated polypeptide of any one of the embodiments, or the diagnostic agent of any one of the embodiments.

### Conditionally Active Protein

The present invention provides a conditionally active protein comprising a heavy chain variable region having three complementarity determining regions having H1, H2, and H3 sequences, wherein:
(a) the H1 sequence is GYTFTTAGX₁Q (SEQ ID NO: 31),
(b) the H2 sequence is WX₂NTHSGEPKYAEDFKG (SEQ ID NO: 32), and
(c) the H3 sequence is X₃GGYAX₄DY (SEQ ID NO: 33);
wherein X₁ is M or D,
   X₂ is I or D,
   X₃ is M or E, and
   X₄ is M or E; and
   a light chain variable region having three complementarity determining regions having L1, L2, and L3 sequences, wherein:
(d) the L1 sequence is KASEDINSYLS (SEQ ID NO: 34),
(e) the L2 sequence is RANRLVD (SEQ ID NO: 35), and
(f) the L3 sequence is LQX₅DEFX₆LT (SEQ ID NO: 36);
wherein X₅ is Y or D and X₆ is P or D;
with the proviso that X₁, X₂, X₃, X₄, X₅ and X₆ cannot be M, I, M, M, Y, P, respectively, at the same time.

In one embodiment of the conditionally active protein, the heavy chain variable region comprises an H1 sequence selected from SEQ ID NOs: 37 and 38, an H2 sequence selected from SEQ ID NOs: 39 and 40, and an H3 sequence selected from SEQ ID NOs: 44-46.

In another embodiment of the conditionally active protein, the heavy chain variable region is selected from:
the heavy chain variable region having the H1 sequence of SEQ ID NO: 37, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 40, and the H3 sequence of SEQ ID NO: 44;
the heavy chain variable region having the H1 sequence of SEQ ID NO: 38, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 39, and the H3 sequence of SEQ ID NO: 45; and
the heavy chain variable region having the H1 sequence of SEQ ID NO: 37, the H2 sequence of SEQ ID NO: 39, and the H3 sequence of SEQ ID NO: 46.

In another embodiment of the conditionally active protein, the heavy chain variable region of the isolated polypeptide has an amino acid sequence selected from the amino acid sequences of SEQ ID NOs: 22-27.

In any one of the embodiments of the conditionally active protein, the light chain variable region has an amino acid sequence selected from the amino acid sequences of SEQ ID NOs: 28-30.

In a further embodiment of the conditionally active protein, the L1 sequence is SEQ NO: 34, the L2 sequence is SEQ ID NO: 35, and the L3 sequence is selected from SEQ ID NOs: 41-43.

In another embodiment of the conditionally active protein, the L1 sequence is SEQ NO: 34, the L2 sequence is SEQ ID NO: 35, and the L3 sequence is selected from SEQ ID NOs: 41-43, and the heavy chain variable region comprises an H1 sequence selected from SEQ ID NOs: 37 and 38, an H2 sequence selected from SEQ ID NOs: 39 and 40, and an H3 sequence selected from SEQ ID NOs: 44-46.

In a further embodiment of the conditionally active protein, the L1 sequence is SEQ NO: 34, the L2 sequence is SEQ ID NO: 35, and the L3 sequence is selected from SEQ ID NOs: 42-43, and the heavy chain variable region is selected from:
the heavy chain variable region having the H1 sequence of SEQ ID NO: 37, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 40, and the H3 sequence of SEQ ID NO: 44;
the heavy chain variable region having the H1 sequence of SEQ ID NO: 38, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 39, and the H3 sequence of SEQ ID NO: 45; and
the heavy chain variable region having the H1 sequence of SEQ ID NO: 37, the H2 sequence of SEQ ID NO: 39, and the H3 sequence of SEQ ID NO: 46.

In another embodiment of the conditionally active protein, the L1 sequence is SEQ ID NO: 34, the L2 sequence is SEQ ID NO: 35, and the L3 sequence is SEQ ID NO: 41; and
the heavy chain variable region is selected from:
the heavy chain variable region having the H1 sequence of SEQ ID NO: 37, the H2 sequence of SEQ ID NO: 40, and the H3 sequence of SEQ ID NO: 44;
the heavy chain variable region having the H1 sequence of SEQ ID NO: 38, the H2 sequence of SEQ ID NO: 39, and the H3 sequence of SEQ ID NO: 44;
the heavy chain variable region having the H1 sequence of SEQ ID NO: 37, the H2 sequence of SEQ ID NO: 39, and the H3 sequence of SEQ ID NO: 45; and
the heavy chain variable region having the H1 sequence of SEQ ID NO: 37, the H2 sequence of SEQ ID NO: 39, and the H3 sequence of SEQ ID NO: 46.

In a further embodiment of the conditionally active protein, the heavy chain variable region has the H1 sequence of SEQ ID NO: 37, the H2 sequence of SEQ ID NO: 39, and the H3 sequence of SEQ ID NO: 44; and the light chain variable region has the L1 sequence of SEQ ID NO: 34, the L2 sequence of SEQ ID NO: 35, and the L3 sequence selected from SEQ ID NOs: 42-43.

In another embodiment of conditionally active protein, the heavy chain variable region has an amino acid sequence of SEQ ID NO: 22; and the light chain variable region has an amino acid sequence selected from the amino acid sequences of SEQ ID NOs: 29-30.

In a still further embodiment of the conditionally active protein, the heavy chain variable region has an amino acid sequence selected from SEQ ID NOs: 23-27; and the light chain variable region has an amino acid sequence of SEQ ID NO: 28.

In another embodiment, the conditionally active protein is selected from:
a conditionally active protein wherein the heavy chain variable region has an amino acid sequence of SEQ ID NO: 23 and the light chain variable region has an amino acid sequence of SEQ ID NO: 28;
a conditionally active protein wherein the heavy chain variable region has an amino acid sequence of SEQ ID NO: 24 and the light chain variable region has an amino acid sequence of SEQ ID NO: 28;
a conditionally active protein wherein the heavy chain variable region has an amino acid sequence of SEQ ID NO: 25 and the light chain variable region has an amino acid sequence of SEQ ID NO: 28;
a conditionally active protein wherein the heavy chain variable region has an amino acid sequence of SEQ ID NO: 26 and the light chain variable region has an amino acid sequence of SEQ ID NO: 28;
a conditionally active protein wherein the heavy chain variable region has an amino acid sequence of SEQ ID NO: 27 and the light chain variable region has an amino acid sequence of SEQ ID NO: 28;
a conditionally active protein wherein the heavy chain variable region has an amino acid sequence of SEQ ID NO: 22 and the light chain variable region has an amino acid sequence of SEQ ID NO: 29; and
a conditionally active protein wherein the heavy chain variable region has an amino acid sequence of SEQ ID NO: 22 and the light chain variable region has an amino acid sequence of SEQ ID NO: 30.

In another embodiment, the conditionally active protein is useful for prevention or treatment of a neurodegenerative disease.

In any one of the embodiments of the conditionally active protein, the conditionally active protein binds to ApoE.

In any one of the embodiments of the conditionally active protein, the conditionally active protein binds to ApoE4.

In any one of the embodiments of the conditionally active protein, the conditionally active protein binds to ApoE3 and/or ApoE4.

In any one of the embodiments of the conditionally active protein, the conditionally active protein binds to ApoE with increased binding activity at an aberrant condition in comparison to the binding activity of the conditionally active protein to ApoE at a normal physiological condition.

In any one of the embodiments of the conditionally active protein, the conditionally active protein binds to ApoE with an increased binding activity at an aberrant condition in comparison to the binding activity of the conditionally active protein to ApoE at a normal physiological condition and the conditionally active protein binds to ApoE with a decreased binding activity at a normal physiological condition in comparison to the binding activity to ApoE of a conditionally active protein having a heavy chain variable region of SEQ ID NO: 22 and a light chain variable region of SEQ ID NO: 28.

In any one of the embodiments of the conditionally active protein, the conditionally active protein is evolved from a parent protein and the binding activity of the conditionally active protein to ApoE at the normal physiological condition is less than the binding activity of the parent protein to ApoE at the normal physiological condition.

In any one of the embodiments of the conditionally active protein, the conditionally active protein binds to ApoE with an increased binding activity at an aberrant pH in a range of from about 5.0 to about 7.0, or from about 5.4 to about 6.8, or from about 5.8 to about 6.8, or from about 6.0 to about 6.8, or from about 6.6 to about 6.8, in comparison to the binding activity to ApoE of the conditionally active protein at a normal physiological condition and the conditionally active protein binds to ApoE with a decreased binding activity at a normal physiological pH in comparison to the binding activity to ApoE of a conditionally active protein having a heavy chain variable region of SEQ ID NO: 22 and a light chain variable region of SEQ ID NO: 28.

In any one of the embodiments of the conditionally active protein, the conditionally active protein binds to ApoE4 with an increased binding activity at an aberrant pH in a range of from about 5.0 to about 7.0, or from about 5.4 to about 6.8, or from about 5.8 to about 6.8, or from about 6.0 to about 6.8, or from about 6.6 to about 6.8, in comparison to the binding activity to ApoE4 of the conditionally active protein at a normal physiological condition and wherein the conditionally active protein binds to ApoE4 with a decreased binding activity at a normal physiological pH in comparison to a conditionally active protein having a heavy chain variable region of SEQ ID NO: 22 and a light chain variable region of SEQ ID NO: 28.

In any one of the embodiments, the normal physiological condition is a pH is in a range of from about 7.2 to about 7.8, or from about 7.2 to about 7.6, or from about 7.2 to about 7.4.

In any one of the embodiments, the conditionally active protein binds to ApoE at the aberrant condition with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

In any one of the embodiments, the conditionally active protein is useful for prevention or treatment of a neurodegenerative disease, and the conditionally active protein binds to ApoE with an increased binding activity at a pH of a dementia brain in comparison with the binding activity to ApoE at a normal physiological pH.

In any one of the embodiments, the conditionally active protein is useful for prevention or treatment of a neurodegenerative disease, and the conditionally active protein has a ratio of a binding activity to ApoE at a pH of a dementia brain to a binding activity to ApoE at a normal physiological pH of at least about 2:1, or at least about 5:1, or at least about 10:1, or at least about 20:1, or at least about 50:1, or at least about 100:1.

In the above embodiment, the pH of the dementia brain is in a range of from about 5.0 to about 7.0, or from about 5.4 to about 6.8, or from about 5.8 to about 6.8, or from about 6.0 to about 6.8, or from about 6.6 to about 6.8. and the normal physiological pH is in a range of from about 7.2 to about 7.8, or from about 7.2 to about 7.6, or from about 7.2 to about 7.4.

In any one of the foregoing embodiments, the conditionally active protein binds to ApoE at the pH of the dementia brain with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

In any one of the embodiments of the conditionally active protein, a therapeutically or prophylactically effective amount of the conditionally active protein reduces ApoE4-amyloid R peptide binding by at least about 10%, at least about 20%, at least about 50%, at least about 90%, compared to the binding between ApoE4 and amyloid β peptide in the absence of the conditionally active protein.

In any one of the embodiments of the conditionally active protein, a therapeutically or prophylactically effective amount of the conditionally active protein reduces C-terminal cleavage of ApoE4 by at least about 5%, at least about 10%, at least about 20%, at least about 50%, at least about 90%, at least about 95%, at least about 99%, compared to the cleavage of ApoE4 in the absence of the conditionally active protein.

In any one of the embodiments of the conditionally active protein, the conditionally active protein binds to amyloid plaques.

In any one of the embodiments, the conditionally active protein comprises at least one non-naturally occurring amino acid.

In any one of the embodiments, the conditionally active protein is glycosylated.

In any one of the embodiments, the conditionally active protein is an antibody or antigen binding antibody fragment.

In any one of the embodiments, the conditionally active protein is a small peptide.

In any one of the embodiments, the conditionally active protein is a cyclic peptide.

In any one of the embodiments, the conditionally active protein is an antibody or antigen binding antibody fragment.

In the embodiment in which the conditionally active protein is an antibody or antigen binding antibody fragment, the conditionally active protein is a multi-specific antibody capable of binding to a receptor on the blood-brain barrier.

In the embodiment in which the conditionally active protein is a multi-specific antibody capable of binding to a receptor on the blood-brain barrier, the binding activity of the conditionally active protein to the receptor on the blood-brain barrier under a blood plasma physiological condition is higher than the same binding activity to the BBB receptor under at least one brain physiological condition.

In the embodiment in which the conditionally active protein is a multi-specific antibody capable of binding to a receptor on the blood-brain barrier, the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

In the embodiment in which the conditionally active protein is a multi-specific antibody capable of binding to a receptor on the blood-brain barrier, the binding activity of the conditionally active protein to the receptor on the blood-brain barrier under a blood plasma physiological condition is higher than the same binding activity to the BBB receptor under at least one brain physiological condition; and the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

In a further embodiment, the present invention provides a diagnostic agent comprising a conditionally active protein of any one of the foregoing embodiments and a detectable label, a chelator or a contrast agent.

In the above embodiment, the diagnostic agent comprises the chelator and the chelator is selected from at least one of ethylenediaminetetraacetic acid, [4-(1,4,8, 11-tetraazacyclotetradec-1-yl) methyljbenzoic acid, cyclohexanediaminetetraacetic acid, ethylenebis(oxyethylenenitrilo)tetraacetic acid, diethylenetriaminepentaacetic acid, citric acid, hydroxyethyl ethylenediamine triacetic acid, iminodiacetic acid, triethylene tetraamine hexaacetic acid, 1,4,7, 10-tetraazacyclododecane-1,4,7, 10-tetra(methylene phosphonic acid), 1,4,8,11-tetraazacyclododecane-1,4,8, 11-tetraacetic acid, 1,4,7, 10-tetraazacyclododecane-1,4,7, 10-tetraacetic acid, and chelating derivatives thereof.

In a further embodiment, the present invention provides a conjugated conditionally active protein comprising any one of the conditionally active proteins, conjugated to a ligand of a receptor on the blood-brain barrier, a polyamine, a therapeutic agent, a prophylactic agent, a diagnostic agent, a detectable label, a chelator or a contrast agent.

In any one of the embodiments, the diagnostic agent comprises the detectable label and the detectable label is selected from at least one of magnetic beads, fluorescent dyes, radiolabels, enzymes, and colorimetric labels.

In any one of the embodiments, the diagnostic agent comprises the contrast agent and the contrast agent is selected from an x-ray contrast agent, gadolinium, dysprosium, and iron.

In a further embodiment, the present invention provides a composition, kit or device comprising any one of the foregoing conditionally active proteins, or any one of the foregoing diagnostic agents.

In yet a further embodiment, the present invention provides a conjugated conditionally active protein comprising any one of the foregoing conditionally active proteins conjugated to a ligand of a receptor on the blood-brain barrier, a polyamine, a therapeutic agent, a prophylactic agent, a diagnostic agent, a detectable label, a chelator or a contrast agent.

In any one of the embodiments of the conjugated conditionally active protein, the conditionally active protein is conjugated to the ligand and the ligand is antibody of the receptor on the blood-brain barrier.

In any one of the embodiments of the conjugated conditionally active protein, the conditionally active protein is conjugated to the ligand and the ligand is a natural ligand of the receptor on the blood-brain barrier or a modified ligand derived from a natural ligand of the receptor on the blood-brain barrier.

In any one of the embodiments of the conjugated conditionally active protein, the conditionally active protein is conjugated to the ligand and the ligand is selected from a peptide having an amino acid sequence of SEQ ID NO: 18, 19, 20, or 21.

In any one of the embodiments of the conjugated conditionally active protein, the conditionally active protein is conjugated to the ligand and the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

In any one of the embodiments of the conjugated conditionally active protein, the conditionally active protein is conjugated to the polyamine.

In any one of the embodiments of the conjugated conditionally active protein, the conditionally active protein is conjugated to the therapeutic agent or the prophylactic agent.

In any one of the embodiments of the conjugated conditionally active protein, the therapeutic or prophylactic agent is selected from at least one of magnesium compounds, anti-excitotoxic compounds, growth factors, agents that bind to beta amyloid protein, calcium channel blockers, calcium chelators, potassium channel blockers, free radical scavengers, antioxidants, GABA agonists, GABA receptor antagonists, glutamate antagonists, NMDA antagonists, NMDA channel blockers, glycine site antagonists, polyamine site antagonists, adenosine receptor antagonists, leukocyte adhesion inhibitors, nitric oxide inhibitors, opioid antagonists, Serotonin agonists, sodium channel blockers, potassium channel openers, anti-inflammatory agents, and protein kinase inhibitors.

In any one of the embodiments of the conjugated conditionally active protein, the therapeutic or prophylactic agent is the growth factor and the growth factor is selected from a Glial cell line derived neurotrophic factor, a brain derived neurotrophic factor, an insulin like growth factor, a fibroblast growth factor, and a neurotrophin.

In any one of the embodiments of the conjugated conditionally active protein, the therapeutic or prophylactic agent is the calcium channel blocker and the calcium channel blocker is selected from nimodipine and flunarizine.

In a further embodiment of the conjugated conditionally active protein, the conjugated conditionally active protein is useful for prevention or treatment of a neurodegenerative disease.

In any one of the embodiments of the conjugated conditionally active protein, the conjugated conditionally active protein binds to ApoE.

In any one of the embodiments of the conjugated conditionally active protein, the conjugated conditionally active protein binds to ApoE4.

In any one of the embodiments of the conjugated conditionally active protein, the conjugated conditionally active protein binds to ApoE3 and/or ApoE4.

In any one of the embodiments of the conjugated conditionally active protein, the conjugated conditionally active protein binds to ApoE with an increased binding activity at an aberrant condition in comparison to the binding activity to ApoE of the conjugated conditionally active protein at a normal physiological condition.

In any one of the embodiments of the conjugated conditionally active protein, the conjugated conditionally active protein binds to ApoE with an increased binding activity at an aberrant condition in comparison to the binding activity of the conjugated conditionally active protein at a normal physiological condition and the conjugated conditionally active protein binds to ApoE with a decreased binding activity at a normal physiological pH in comparison to the binding activity to ApoE of a conjugated conditionally active protein having a heavy chain variable region of SEQ ID NO: 22 and a light chain variable region of SEQ ID NO: 28.

In any one of the embodiments of the conjugated conditionally active protein, the conditionally active protein of the conjugated conditional active protein is evolved from a parent protein and the binding activity of the conjugated conditionally active protein to ApoE at the normal physiological condition is less than the binding activity of the parent protein to ApoE at the normal physiological condition.

In any one of the embodiments of the conjugated conditionally active protein, the conjugated conditionally active protein binds to ApoE with an increased binding activity at an aberrant pH in a range of from about 5.0 to about 7.0, or from about 5.4 to about 6.8, or from about 5.8 to about 6.8, or from about 6.0 to about 6.8, or from about 6.6 to about 6.8, in comparison to the binding activity of the conjugated conditionally active protein to ApoE at a normal physiological pH and the conjugated conditionally active protein binds to ApoE with a decreased binding activity at a normal physiological pH in comparison to the binding activation to ApoE of a conjugated conditionally active protein having a heavy chain variable region of SEQ ID NO: 22 and a light chain variable region of SEQ ID NO: 28.

In any one of the embodiments of the conjugated conditionally active protein, the conjugated conditionally active protein binds to ApoE with an increased binding activity at an aberrant pH in a range of from about 5.0 to about 7.0, or from about 5.4 to about 6.8, or from about 5.8 to about 6.8, or from about 6.0 to about 6.8, or from about 6.6 to about 6.8, in comparison to the binding activity of the conjugated conditionally active protein to ApoE at a normal physiological condition, the conjugated conditionally active protein binds to ApoE with a decreased binding activity at a normal physiological pH in comparison to the binding activity to ApoE of a conjugated conditionally active protein having a heavy chain variable region of SEQ ID NO: 22 and a light chain variable region of SEQ ID NO: 28, and the conjugated conditionally active protein is evolved from a parent protein and the binding activity of the conjugated conditionally active protein to ApoE at the normal physiological condition is less than the binding activity of the parent protein to ApoE at the normal physiological condition.

In any one of the embodiments, the normal physiological condition is a pH in a range of from about 7.2 to about 7.8, or from about 7.2 to about 7.6, or from about 7.2 to about 7.4.

In any one of the embodiments, the conjugated conditionally active protein binds to ApoE at the aberrant condition with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

In any one of the embodiments of the conjugated conditionally active protein in which the conjugated conditionally active protein is useful for prevention or treatment of a neurodegenerative disease, the conjugated conditionally active protein binds to ApoE with an increased binding activity at a pH of a dementia brain in comparison with the binding activity to ApoE at a normal physiological pH.

In any one of the embodiments in which the conjugated conditionally active protein is useful for prevention or treatment of a neurodegenerative disease, the conjugated conditionally active protein has a ratio of a binding activity to ApoE at a pH of a dementia brain to a binding activity to ApoE at a normal physiological pH of at least about 2:1, or at least about 5:1, or at least about 10:1, or at least about 20:1, or at least about 50:1, or at least about 100:1.

In the above embodiment, the pH of the dementia brain is in a range of from about 5.0 to about 7.0, or from about 5.4 to about 6.8, or from about 5.8 to about 6.8, or from about 6.0 to about 6.8, or from about 6.6 to about 6.8. and the normal physiological pH is in a range of from about 7.2 to about 7.8, or from about 7.2 to about 7.6, or from about 7.2 to about 7.4.

In any one of the embodiments, the conjugated conditionally active protein binds to ApoE at the pH of the dementia brain with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

In any one of the embodiments of the conjugated conditionally active protein, a therapeutically or prophylactically effective amount of the conjugated conditionally active protein reduces ApoE4-amyloid β peptide binding by at least about 10%, at least about 20%, at least about 50%, at least about 90%, compared to the binding between ApoE4 and amyloid β peptide in the absence of the conditionally active protein.

In any one of the embodiments of the conjugated conditionally active protein, a therapeutically or prophylactically effective amount of the conjugated conditionally active protein reduces C-terminal cleavage of ApoE4 by at least about 5%, at least about 10%, at least about 20%, at least about 50%, at least about 90%, at least about 95%, at least about 99%, compared to the cleavage of ApoE4 in the absence of the conditionally active protein.

In any one of the embodiments, the conditionally active protein binds to amyloid plaques.

In any one of the embodiments, the conditionally active protein comprises at least one non-naturally occurring amino acid.

In any one of the embodiments, the conditionally active protein is glycosylated.

In any one of the embodiments, the conditionally active protein is an antibody or antigen binding antibody fragment.

In any one of the embodiments, the conditionally active protein is a small peptide.

In the embodiment in which the conjugated conditionally active protein is an antibody or antigen binding antibody fragment, the conjugated conditionally active protein is a multi-specific antibody capable of binding to a receptor on the blood-brain barrier.

In the embodiment in which the conjugated conditionally active protein is a multi-specific antibody capable of binding to a receptor on the blood-brain barrier, the binding activity of the conjugated conditionally active protein to the receptor on the blood-brain barrier under a blood plasma physiological condition is higher than the same binding activity to the BBB receptor under at least one brain physiological condition.

In the embodiment in which the conjugated conditionally active protein is a multi-specific antibody capable of binding to a receptor on the blood-brain barrier, the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

In the embodiment in which the conjugated conditionally active protein is a multi-specific antibody capable of binding to a receptor on the blood-brain barrier, the binding activity of the conjugated conditionally active protein to the receptor on the blood-brain barrier under a blood plasma physiological condition is higher than the same binding activity to the BBB receptor under at least one brain physiological condition; and the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

In a further embodiment, the present invention provides a diagnostic agent comprising a conjugated conditionally active protein of any one of the foregoing embodiments and a detectable label, a chelator or a contrast agent.

In any one embodiment, the diagnostic agent comprises the chelator and the chelator is selected from at least one of ethylenediaminetetraacetic acid, [4-(1,4,8, 11-tetraazacyclotetradec-1-yl) methyljbenzoic acid, cyclohexanediaminetetraacetic acid, ethylenebis(oxyethylenenitrilo)tetraacetic acid, diethylenetriaminepentaacetic acid, citric acid, hydroxyethyl ethylenediamine triacetic acid, iminodiacetic acid, triethylene tetraamine hexaacetic acid, 1,4,7, 10-tetraazacyclododecane-1,4,7, 10-tetra(methylene phosphonic acid), 1,4,8,11-tetraazacyclododecane-1,4,8, 11-tetraacetic acid, 1,4,7, 10-tetraazacyclododecane-1,4,7, 10-tetraacetic acid, and chelating derivatives thereof.

In a further embodiment, the present invention provides a diagnostic agent comprising the conditionally active protein of any one of the embodiments of the conditionally active protein, conjugated to a ligand of a receptor on the blood-brain barrier, a polyamine, a therapeutic agent, a prophylactic agent, a diagnostic agent, a detectable label, a chelator or a contrast agent.

In any one embodiment of the diagnostic agent, the diagnostic agent comprises the detectable label and the detectable label is selected from at least one of magnetic beads, fluorescent dyes, radiolabels, enzymes, and colorimetric labels.

In any one embodiment of the diagnostic agent, the diagnostic agent comprises the contrast agent and the contrast agent is selected from an x-ray contrast agent, gadolinium, dysprosium, and iron.

In another further embodiment, the present invention provides a composition, kit or device comprising a conjugated conditionally active protein of any one of the embodiments for a conjugated conditionally active protein, or a diagnostic agent of any one of the embodiments. The present invention provides a conditionally active protein that binds to ApoE4. The conditionally active protein binds to ApoE3 and/or ApoE4 with an increased binding activity at an acidic pH as present in a dementia brain in comparison with the binding activity to the ApoE3 and/or ApoE4 at a normal physiological pH, e.g. as present in blood. In some embodiments, the conditionally active protein is an antibody or antibody fragment. In some other embodiments, the conditionally active protein is a small peptide. In one embodiment, the conditionally active protein is a cyclic peptide.

The dementia brain has an acidic pH that is related to (e.g., caused by) the formation of amyloid plaques that are common in the brains of patients suffering from many different neurodegenerative diseases (Su et al., “Acidic pH promotes the formation of toxic fibrils from beta-amyloid peptide,” Brain Res., vol. 893, pp. 287-291, 2001). For example, senile plaques in the neocortical region in the brain of AD patients may generate a local pH as low as 5.4. Lower pH is one of the major factors that determine aggregation rates and fibril morphologies of amyloid R. See Bin, “Amyloid-β peptide (1-42) aggregation induced by copper ions under acidic conditions,” Acta Biochimica et Biophysica Sinica, vol. 45, pp. 570-577, 2013.

For example, Yates et al. measured the pH in the postmortem brain of patients diagnosed with Alzheimer's disease and other dementia patients and found an acidic pH in the range of from 6.5 to 6.8 (Yates et al., “Enzyme activities in relation to pH and lactate in postmortem brain in Alzheimer-type and other dementias,” J. Neurochem., vol. 55, pp. 1624-1630, 1990). Indeed, it has been repeatedly observed that acidosis in brains, i.e., lowering of the pH in the brain, is an important pathological cause of neuron death and loss of cognitive function, which are associated with neurodegenerative diseases (Gonzales and Sumien, “Acidity and acid-sensing ion channels in the normal and Alzheimer's disease brain,” J Alzheimers Dis., vol. 57, pp. 1137-1144, 2017; Humpel, “Chronic mild cerebrovascular dysfunctional as a cause for Alzheimer's disease,” Experimental Gerontology, vol. 46, pp. 225-232, 2011).

The acidic pH in a dementia brain may be in the range of from about 5.0 to about 7.0, or from about 5.2 to about 6.8, or from about 5.4 to about 6.8, or from about 5.6 to about 6.8, or from about 5.8 to about 6.8, or from about 6.0 to about 6.8, or from about 6.2 to about 6.8, or from about 6.4 to about 6.8, or from about 6.6 to about 6.8. In some embodiments, the pH in a dementia brain may be in the range of from about 6.4 to about 7.0, or from about 6.6 to about 7.0, or from about 6.8 to about 7.0.

The normal physiological pH in the blood is well-established in the art. In some embodiments, the normal physiological pH in the blood may be in the range of from about 7.0 to about 7.8, or from about 7.1 to about 7.7, or from about 7.2 to about 7.6, or from about 7.2 to about 7.5, or from about 7.2 to about 7.4.

Based on the differences in the sequences of particular regions of ApoE2, ApoE3, and ApoE4, the prior art has developed antibodies that bind to the regions that are specific to ApoE4, and thus do not exist in ApoE2 (the non-risk factor). It is known that the sequences of ApoE2, ApoE3 and ApoE4 differ only at positions 112 and 158, where ApoE2 has Cys112 and Cys158; ApoE3 has Cys112 and Arg158; and ApoE4 has Arg112 and Arg158. The regions around the Arg112 and Arg158 in the ApoE4 are the epitopes typically selected in the prior art for generating antibodies that specifically target ApoE4, without binding or with lesser binding to ApoE3 and ApoE2. The antibodies of the prior art thus preferentially bind to ApoE4 relative to ApoE3 and/or ApoE2.

ApoE4 is also expressed in normal tissues and participates in cholesterol metabolism and lipid homeostasis by mediating lipid transport. The antibodies of the prior art developed to bind the ApoE4, will also bind ApoE4 present in normal tissues thereby disrupting important cholesterol metabolism and lipid transport.

The present inventors have found that the two histidine residues of ApoE4 located at positions 140 and 299 become at least partially positively charged at the pH levels present in dementia brains. Specifically, due to the acidic pH in the dementia brain the imidazole ring located on a side chain of histidine acquires a proton (H⁺) because this imidazole ring has an isoelectric point (pI) at pH 6.04:

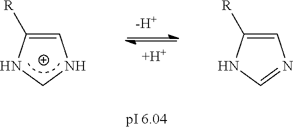

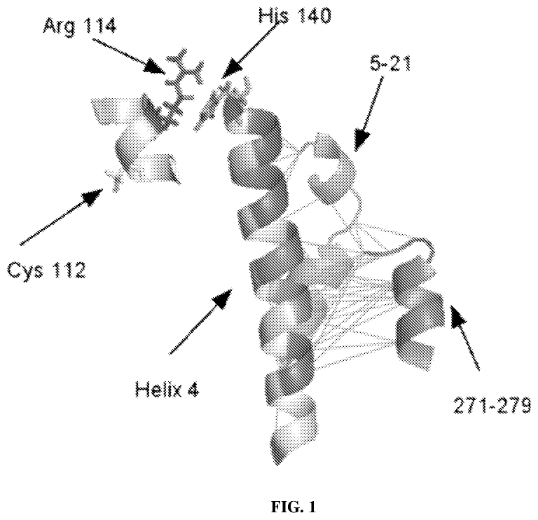

The conditionally active proteins of the present invention are targeted to bind to one of the two regions in ApoE4 that comprise a histidine residue. One of these regions has SEQ ID NO:4 and encompasses histidine at position 140 and the other of these regions has SEQ ID NO:5 and encompasses histidine at position 299. See FIG. 1. The conditionally active proteins have an increased binding activity to ApoE4 at a pH in the dementia brain in comparison with the binding activity to the same region in a normal physiological pH in the blood. The conditionally active protein may, for example, bind to the region of ApoE4 with a positively charged histidine residue at the pH in a dementia brain, in comparison with the same region where the histidine residue is not charged at the normal physiological pH in the blood.

### Methods of Generating

The present invention provides a method of generating, from a parent protein with a known binding activity to ApoE at a normal physiological pH or whose activity to ApoE at a normal physiological condition is determined, a conditionally active protein for prevention or treatment of a neurodegenerative disease, comprising steps of:
a) mutating the parent protein to generate a set of mutant proteins;
b) subjecting the set of mutant proteins to a first assay at a pH of a dementia brain and a second assay at a normal physiological pH; and
c) selecting the conditionally active protein from the set of mutant proteins of step b) that has an increased binding activity to ApoE in the first assay in comparison to the binding activity to ApoE in the second assay and which have a decreased binding activity to ApoE at a normal physiological pH in comparison to the parent protein.

In the methods, the pH in the dementia brain may be in a range of from about 5.0 to about 7.0, or from about 5.4 to about 6.8, or from about 5.6 to about 6.8, or from about 6.0 to about 6.8, or from about 6.4 to about 6.8, and the normal physiological pH may be in a range of from about 7.2 to about 7.8, or from about 7.2 to about 7.6, or from about 7.2 to about 7.4.

In any one of the methods, assay solutions for the first and second assays may contain at least one component selected from at least one of:
(i) an inorganic compound,
(ii) an ion selected from a magnesium ion, a sulfate ion, a bisulfate ion, a carbonate ion, a bicarbonate ion, a nitrate ion, a nitrite ion, a phosphate ion, a hydrogen phosphate ion, a dihydrogen phosphate ion, a persulfate ion, a monopersulfate ion, a borate ion, an ammonium ion, a phosphorus ion, a sulfur ion, a chloride ion, a magnesium ion, a sodium ion, a potassium ion, an ammonium ion, an iron ion, a zinc ion, and a copper ion, and
(iii) an organic molecule other than polypeptides of SEQ ID NO. 4 and 5.

In the methods, the at least one component (i)-(iii) may have substantially the same concentration in the assay solutions for both the first and second assays.

In any of the methods, the at least one component may comprise an inorganic compound selected from at least one of boric acid, calcium chloride, calcium nitrate, di-ammonium phosphate, magnesium sulfate, mono-ammonium phosphate, mono-potassium phosphate, potassium chloride, potassium sulfate, copper sulfate, iron sulfate, manganese sulfate, zinc sulfate, magnesium sulfate, calcium nitrate, calcium chelate, copper chelate, iron chelate, iron chelate, manganese chelate, zinc chelate, ammonium molybdate, ammonium sulphate, calcium carbonate, magnesium phosphate, potassium bicarbonate, potassium nitrate, hydrochloric acid, carbon dioxide, sulfuric acid, phosphoric acid, carbonic acid, uric acid, hydrogen chloride, and urea.

In any one of the methods, the at least one component may be selected from one or more of uric acid in concentration range of 2-7.0 mg/dL, calcium ion in a concentration range of 8.2-11.6 mg/dL, chloride ion in a concentration range of 355-381 mg/dL, iron ion in a concentration range of 0.028-0.210 mg/dL, potassium ion in a concentration range of 12.1-25.4 mg/dL, sodium ion in a concentration range of 300-330 mg/dL, and carbonic acid in a concentration range of 15-30 mM.

In any one of the methods, the ion may be selected from at least one of magnesium ion, sulfate ion, bisulfate ion, carbonate ion, bicarbonate ion, nitrate ion, nitrite ion, phosphate ion, hydrogen phosphate ion, dihydrogen phosphate ion, persulfate ion, monopersulfate ion, borate ion, and ammonium ion.

Suitable methods for generating a conditionally active protein from a parent protein have been described in WO 2016/138071. The parent protein may be an antibody that itself may either be a monoclonal antibody or a polyclonal antibody, or the parent protein may be an antibody fragment that binds to ApoE3 and/or ApoE4. The parent antibody may be generated by immunization with the region using methods of immunization, producing and isolating antibodies known to those of skill in the art and described in the literature, see, e.g., Coligan, Current Protocols In Immunology, Wiley/Greene, NY (1991); Stites (eds.) Basic And Clinical Immunology (7^{th} ed.) Lange Medical Publications, Los Altos, Calif. (“Stites”); Goding, Monoclonal Antibodies: Principles and Practice (2d ed.) Academic Press, New York, N. Y. (1986); Kohler (1975) “Continuous cultures of fused cells secreting antibody of predefined specificity”, Nature 256:495; Harlow (1988) Antibodies, A Laboratory Manual, Cold Spring Harbor Publications, New York.

Parent antibodies can also be generated in vitro, e.g., using recombinant antibody binding site expressing phage display libraries, in addition to the traditional in vivo methods using animals. See, e.g., Hoogenboom, “Designing and optimizing library selection strategies for generating high-affinity antibodies”, Trends Biotechnol., vol. 15, pp. 62-70, 1997, and Katz, “Structural and mechanistic determinants of affinity and specificity of ligands discovered or engineered by phage display”, Annu. Rev. Biophys. Biomol. Struct., vol. 26, pp. 27-45, 1997.

In some embodiments, the parent protein may be a small peptide or a cyclic peptide. A small peptide or a cyclic peptide that binds to ApoE3 and/or ApoE4 may be screened from a library of small peptides and/or cyclic peptides. Methods of construction and screening of small peptide libraries have been described in U.S. Pat. No. 5,733,731. Further, peptide libraries can also be constructed by synthesizing a large number of distinct peptides. For example, the peptide library may contain all possible combinations of the amino acids to increase the possibility of finding a small peptide that binds to ApoE3 and/or ApoE4. There are 20 natural occurring amino acids, thus 8,000 tripeptides need to be synthesized to construct a complete tripeptide library, 160,000 tetrapeptides need to be synthesized to construct a complete tetrapeptide library, 3,200,000 pentapeptides need to be synthesized to construct a complete pentapeptide library, and 64,000,000 hexapeptides need to be synthesized to construct a complete hexapeptide library. The synthesis of small peptides may be based on the solid phase synthesis method described in Furka et al., “General method for rapid synthesis of multicomponent peptide mixtures,” Int. J. Peptide Protein Res., 1991, 37, 487-493.

In another example, the library of small peptides may be displayed on the surface of phages for screening as described in Smith, “Filamentous fusion phage: novel expression vectors that display cloned antigens on the virion surface,” Science, vol., 228, pp. 1315-1317 (1985). In some embodiments, combinatorial peptide libraries as described in WO 2005/035552 may be used.

In some other embodiments, the parent protein is a cyclic peptide that binds to ApoE3 and/or ApoE4. The cyclic peptide may be screened from a cyclic peptide library such as that described in WO 19998/054577, WO 1995/001800 and WO 2001/066565.

In some embodiments, the parent protein may be a fragment of a naturally occurring protein. For example, the parent protein may be the catalytic domain of an enzyme, the binding domain of a ligand or receptor, or the variable region of an antibody.

In some embodiments, the parent protein may be a therapeutic or prophylactic protein or a biosimilar. The therapeutic or prophylactic protein may be an antibody, a protein, a small peptide, or a cyclic peptide that is administered to a subject to treat, prevent or ameliorate a disease or condition or to improve health of the subject. For one example, the therapeutic or prophylactic protein may be a human protein. In another example, the therapeutic or prophylactic protein may be approved for therapeutic or prophylactic use for humans or animals by a regulatory agency in a country or region such as U.S. Food and Drug Administration and European Medicines Agency.

A biosimilar refers to a biopharmaceutical which is deemed to be comparable in quality, safety, and efficacy to a reference biologic product marketed by an innovator pharmaceutical company (as defined in Section 351(i) of the Public Health Service Act (42 U.S.C. 262(i) in the U.S.). There may be minor differences in clinically inactive components between the biosimilar and the reference biologic product.

Starting from the parent protein, the method of WO 2016/138071 may be used to generate the conditionally active protein. Briefly, this method comprises the steps of:
(i) mutating the parent protein to generate a set of mutant proteins;
(ii) subjecting the set of mutant proteins to a first assay at a pH in the dementia brain and a second assay at a normal physiological pH; and
(iii) selecting the conditionally active protein from the set of mutant proteins that has an increased binding activity to ApoE3 and/or ApoE4 in the first assay in comparison to the binding activity in the second assay (with the histidine residue not charged).

The assay solutions for the first and second assays preferably include a buffer selected from citrate buffers such as sodium citrate, phosphate buffers, bicarbonate buffers such as the Krebs buffer, phosphate buffered saline (PBS) buffer, Hank's buffer, Tris buffer, HEPES buffer, etc. Other buffers known to a person skilled in the art to be suitable for the assays may also be used.

The assay solutions of the invention may contain at least one molecule selected from inorganic compounds, ions and organic molecules, preferably ones that are commonly found in a bodily fluid of a mammal such as a human or animal. These inorganic compounds, ions and organic molecules are described in detail in WO 2016/138071.

In some embodiments, the inorganic compounds or ions may be selected from one or more of boric acid, calcium chloride, calcium nitrate, di-ammonium phosphate, magnesium sulfate, mono-ammonium phosphate, mono-potassium phosphate, potassium chloride, potassium sulfate, copper sulfate, iron sulfate, manganese sulfate, zinc sulfate, calcium nitrate, calcium chelate, copper chelate, iron chelate, manganese chelate, zinc chelate, ammonium molybdate, ammonium sulphate, calcium carbonate, magnesium phosphate, potassium bicarbonate, potassium nitrate, hydrochloric acid, carbon dioxide, sulfuric acid, phosphoric acid, carbonic acid, uric acid, hydrogen chloride, urea, phosphorus ion, sulfuric ion, chloride ion, magnesium ion, sodium ion, potassium ion, ammonium ion, iron ion, zinc ion and copper ion.

Examples of normal physiological concentrations of some of the inorganic compounds include: uric acid in a concentration range of 2-7.0 mg/dL, calcium ion in a concentration range of 8.2-11.6 mg/dL, chloride ion in a concentration range of 355-381 mg/dL, iron ion in a concentration range of 0.028-0.210 mg/dL, potassium ion in a concentration range of 12.1-25.4 mg/dL, sodium ion in a concentration range of 300-330 mg/dL, carbonic acid in a concentration range of 15-30 mM, citrate ion at about 80 μM, histidine ion in the range of 0.05-2.6 mM, histamine in the range of 0.3-1 μM, HAPT ion (hydrogenated adenosine triphosphate) in the range of 1-20 μM, and HADP ion in the range of 1-20 μM.

In some embodiments, the ion present in the assay solutions for both the normal physiological condition and the aberrant condition is selected from hydroxide ion, halide ion (chloride, bromide, iodide), oxyhalide ion, sulfate ion, magnesium ion, calcium ion, bisulfate ion, carbonate ion, bicarbonate ion, sulfonate ion, oxyhalide ion, nitrate ion, nitrite ion, phosphate ion, hydrogen phosphate ion, dihydrogen phosphate ion, persulfate ion, monopersulfate ion, borate ion, ammonium ion, or organic ion, such as carboxylate ion, phenolate ion, sulfonate ion (organosulfate such as methyl sulfate), vanadate ion, tungstate ion, borate ion, organoboronate ion, citrate ion, oxalate ion, acetate ion, pentaborate ion, histidine ion, and phenolate ion.

The conditionally active protein may interact with a molecule selected from inorganic compounds, ions, and organic molecules. Such interactions between the conditionally active protein and the molecule may include hydrogen bonding, hydrophobic interactions, and Van der Waals interactions.

In some embodiments, a molecule in the assay solution may have a pKa between the lower pH of the dementia brain and the normal physiological pH. The pKa of the molecule in the assay solution may be up to 0.5, 1, 1.5, 2, 2.5, or 3 units above the pH of the dementia brain. This molecule preferably has a molecular weight of less than 900 a.m.u. and may preferably be selected from histidine, histamine, hydrogenated adenosine diphosphate, hydrogenated adenosine triphosphate, citrate, bicarbonate, acetate, lactate, bisulfide, hydrogen sulfide, ammonium, dihydrogen phosphate and any combination thereof.

Certain conditionally active proteins contain an increased number (or proportion) of charged amino acid residues in comparison to the amino acid residues of the parent protein from which the conditionally active proteins are derived. There are three positively charged amino acid residues: lysine, arginine and histidine; and two negatively charged amino acid residues: aspartate and glutamate. These charged amino acid residues are over-represented in certain conditionally active proteins in comparison with the parent protein from which the conditionally active proteins are derived. As a result, the conditionally active proteins are more likely to interact with the charged molecule in the assay solutions since the number of charged amino acid residues in the conditionally active proteins has increased. This, in turn, influences the activity of the conditionally active proteins.

Certain conditionally active proteins typically have different activities dependent on the molecule(s) in the assay solution. Some of the molecules that may lead to such different activities may have at least two different ionization states: an uncharged or less charged state at one pH and a charged or more charged state at a different pH. The presence of these different ionization states may alter the activity of the conditionally active protein. The charged or more charged state of the molecule may increase the interaction of the molecule with charged amino acid residues in the conditionally active proteins. This mechanism may be employed to enhance the selectivity and/or pH-dependent activity of the conditionally active proteins.

The location of the charged amino acid residues on the conditionally active proteins may also have an influence on the activity. For example, the proximity of charged amino acid residues to a binding site of the conditionally active proteins may be used to influence the activity of the conditionally active proteins.

In some embodiments, it may be the case that the interaction of the charged molecules with the conditionally active proteins may form salt bridges between different moieties on the protein, especially the moieties that are charged or polarized. The formation of salt bridges is known to stabilize polypeptide structures (Donald, et al., “Salt Bridges: Geometrically Specific, Designable Interactions,” Proteins, 79(3): 898-915, 2011; Hendsch, et al., “Do salt bridges stabilize proteins? A continuum electrostatic analysis,” Protein Science, 3:211-226, 1994). The salt bridges can stabilize or fix the protein structure which normally undergoes constant minor structural variation called “breathing” (Parak, “Proteins in action: the physics of structural fluctuations and conformational changes,” Curr Opin Struct Biol., 13(5):552-557, 2003). The protein structural “breathing” is important for protein function and its binding with its partner because the structural fluctuation permits the conditionally active protein to efficiently recognize and bind to its partner (Karplus, et al., “Molecular dynamics and protein functions,” PNAS, vol. 102, pp. 6679-6685, 2015). By forming salt bridges, the binding site, especially the binding pocket, on the conditionally active protein may be less accessible to its partner, possible because the salt bridges may directly block the partner from accessing the binding site. Even with salt bridges remote from the binding site, the allosteric effect may alter the conformation of the binding site to inhibit binding. Therefore, after the salt bridges stabilize (fix) the structure of the conditionally active protein, the protein may become less active in binding to its partner, leading to decreased activity in a particular environment that favors formation of such salt bridges.

One known example of the stabilization of a protein structure by salt bridges occurs with hemoglobin. Structural and chemical studies have revealed that at least two sets of chemical groups are responsible for the salt bridges: the amino termini and the side chains of histidines β146 and α122, which have pKa values near pH 7. In deoxyhemoglobin, the terminal carboxylate group of β146 forms a salt bridge with a lysine residue in the a subunit of the other up dimer. This interaction locks the side chain of histidine β146 in a position where it can participate in a salt bridge with negatively charged aspartate 94 in the same chain, provided that the imidazole group of the histidine residue is protonated. At a high pH, the side chain of histidine β146 is not protonated and the salt bridges do not form. As the pH drops, however, the side chain of histidine β146 becomes protonated and the salt bridge between histidine β146 and aspartate β94 forms, thereby stabilizing the quaternary structure of deoxyhemoglobin, leading to a greater tendency for oxygen to be released at actively metabolizing tissues (with lower pH). The hemoglobin shows a pH-dependent binding activity for oxygen where at a low pH, the binding activity for oxygen is reduced because of the formation of salt bridges. On the other hand, at a high pH, the binding activity for oxygen is increased due to the absence of these salt bridges.

Similarly, the molecules such as bicarbonate may reduce the binding activity of the conditionally active protein to its partner by forming salt bridges in the conditionally active protein. For example, at a pH lower than its pKa of 6.4, bicarbonate is protonated and thus not charged. The uncharged bicarbonate is not capable of forming salt bridges, and thus has little effect on the binding of the conditionally active protein with its partner. Hence, the conditionally active protein has high binding activity with its partner at the low pH. On the other hand, at a higher pH greater than the pKa of 6.4, bicarbonate is ionized by losing the proton, thus becoming negatively charged. The negatively charged bicarbonate will form salt bridges between positively charged moieties or polarized moieties on the conditionally active protein to thereby stabilize the structure of the conditionally active protein. This will block or reduce the binding of the conditionally active protein with its partner. Hence the conditionally active protein is caused to have a lower activity at the higher pH by the presence of the bicarbonate. The conditionally active protein thus has a pH-dependent activity in the presence of bicarbonate with higher binding activity at a low pH than at a higher pH.

When a molecule such as bicarbonate is absent from the assay solution, the conditionally active protein may lose its conditional activity. This is likely due to the lack of salt bridges on the conditionally active protein to stabilize (fix) the structure of the protein. As a result, the binding partner will have similar access to the binding site on the conditionally active protein at any pH, thereby providing a similar activity at both the lower pH and the higher pH.

It is to be understood that, though the salt bridges (ionic bonds) are the strongest and most common manner for molecules to affect the activity of the conditionally active proteins, other interactions between such molecules and the conditionally active proteins such as hydrogen bonding, hydrophobic interactions, and van der Waals interactions may also contribute to stabilize (fix) the structure of the conditionally active proteins.

In some embodiments, in order to select a suitable molecule, the conditionally active protein is compared with the parent protein from which it is evolved to determine whether the conditionally active protein has a higher proportion of negatively charged amino acid residues or positively charged amino acid residues. A molecule with a suitable charge at the normal physiological pH may then be chosen to influence the activity of the conditionally active protein. For example, when the conditionally active protein has a higher proportion of positively charged amino acid residues than the parent protein, the suitable molecule should typically be negatively charged at the normal physiological pH so as to interact with the conditionally active protein at that condition. On the other hand, when the conditionally active protein has a higher proportion of negatively charged amino acid residues than the parent protein, the suitable molecule should typically be positively charged at the normal physiological pH so as to interact with the conditionally active protein at that condition.

Thus, a suitable molecule may be an inorganic or organic molecule that transits from an uncharged or less charged state at the lower pH of the dementia brain to charged or more charged state at the normal physiological pH. The molecule should typically have a pKa between the lower pH and the normal physiological pH. For example, bicarbonate has pKa at 6.4. Thus, at a higher pH such as pH 7.4, the negatively charged bicarbonate will bind to the positively charged amino acid residues in the conditionally active proteins and reduce the activity. On the other hand, at a lower pH such as pH 6.0-6.2, the less charged bicarbonate will not bind in the same quantity to the conditionally active proteins thus allowing a higher activity of the conditionally active proteins.

Bisulfide has a pKa 7.05. Thus, at a higher pH such as pH 7.4, the more negatively charged bisulfide will bind to the positively charged amino acid residues in the conditionally active proteins and reduce the activity. On the other hand, at a lower pH such as pH 6.0-6.8, the less charged hydrogen sulfide/bisulfide will not bind at the same level to the conditionally active proteins thus allowing a higher activity of the conditionally active proteins.

Exemplary molecules are bisulfide, hydrogen sulfide, histidine, histamine, citrate, bicarbonate, acetate, and lactate. Each of these are small molecules having a pKa between 6.2 and 7.0. Other suitable small molecules may be found in textbooks such as the CRC Handbook of Chemistry and Physics, 96th Edition, by CRC press, 2015; Chemical Properties Handbook, McGraw-Hill Education, 1998 using the principles described in the present application.

The molecules preferably have a low molecular weight and/or a relatively small conformation to ensure maximum access to small pockets on the conditionally active protein by minimizing steric hindrance. For this reason, such small molecules typically have a molecular weight of less than 900 a.m.u., or less than 500 a.m.u., or less than 200 a.m.u., or less than 100 a.m.u. For example, hydrogen sulfide, bisulfide and bicarbonate each have low molecular weights and small structures that provide access to the small binding pockets of conditionally active proteins.

The concentration of the molecules in the assay solutions is typically at or near the physiological concentration of the molecules in a subject. For example, the physiological concentration of bicarbonate (in human serum) is in the range of 15 to 30 mM. Thus, the concentration of bicarbonate in the assay solutions may be from 10 mM to 40 mM, or from 15 mM to 30 mM, or from 20 mM to 25 mM, or about 20 mM. The physiological concentration of bisulfide is also low. The concentration of bisulfide in the assay solutions may be from 3 to 500 nM, or from 5 to 200 nM, or from 10 to 100 nM, or from 10 to 50 nM.

The molecules may be present in the assay solution simulating the lower pH in the dementia brain and the assay solution simulating the normal physiological pH at substantially the same concentration, e.g. about 20 μM for bicarbonate.

In some embodiments, the conditionally active protein is pH-dependent when two or more different small molecules are present. For example, a combination of bicarbonate and histidine may be employed for this purpose. In such case, the two or more small molecules would be present in the assay solutions.

The molecules in the assay solutions may be formed in situ from a component of the assay solutions or be directly included in the assay solutions. For example, CO₂ from the air may dissolve in the assay solutions to provide bicarbonate as the species in the assay solutions. In another example, sodium dihydrogen phosphate may be added to the assay solution to provide dihydrogen phosphate as the species in the assay solutions. When the molecule is absent, the conditionally active proteins may lose their pH-dependent activity. Thus, in the absence of the molecule, the conditionally active proteins may have similar activity at the lower pH of dementia brain and at the normal physiological pH.

In some embodiments, the conditionally active protein shows an increased activity at the lower pH of the dementia brain in comparison with at the normal physiological pH, in the presence of an ancillary protein. The ancillary protein may be a protein present in blood or serum of a human or animal. One suitable protein may be albumin, particularly mammalian albumin, such as bovine albumin or human albumin.

In one aspect, the ancillary protein such as albumin may be present in the assay solutions used for screening and selecting the conditionally active protein from the mutant proteins produced by the evolving step. In another aspect, the assay solutions with the ancillary protein such as albumin are also used for screening and selecting the conditionally active protein under the same or different conditions. In yet another embodiment, the assay solutions do not contain any serum or ancillary protein from serum of a human or animal.

In some embodiments, two or more of the foregoing inorganic compounds, ions, and organic molecules are added at substantially the same concentrations to both the assay solution simulating the normal physiological pH and the assay solution simulating the lower pH in the dementia brain. For example, both bicarbonate and histidine can be added to both assay solutions. The bicarbonate concentration may be different between the two assay solutions, while the histidine may be added at the same concentration in both assay solutions. The concentrations of the components in the assay solutions may be selected based on actual concentrations of the same components found in the dementia brain environment and the normal blood environment, for example.

In one embodiment, human serum may be added to both assay solutions at substantially the same concentration. Since human serum has a large number of inorganic compounds, ions, and organic molecules (including proteins), the assay solutions will have multiple components selected from inorganic compounds, ions, organic molecules presented at substantially the same concentrations in both assay solutions.

In some embodiments, the assay solutions may be designed for selecting conditionally active biological proteins with an activity dependent on two or more conditions. In one exemplary embodiment, the conditionally active protein may have activity dependent on both pH and bicarbonate concentration. The assay solutions for selecting such a conditionally active protein may be an assay solution for the normal physiological pH 7.2-7.6, an assay solution for the normal bicarbonate concentration in the range of from 25 to 30 mM, an assay solution for the lower pH of the dementia brain may with a pH at 5.0-6.8, and an assay with bicarbonate at a concentration in the range of from 10 to 20 mM.

Optionally, the assay solutions for both normal physiological pH and lower pH of dementia brain may also comprise an ion to assist the binding between the conditionally active proteins and ApoE4, in order to increase the number of hits for conditionally active proteins.

In some embodiments, certain components of serum may be purposely minimized or omitted from the assay solutions. For example, when screening antibodies, components of serum that bind with or adsorb proteins can be minimized or omitted from the assay solutions. Such bound proteins may give false positives thereby including bound mutant proteins that are not conditionally active but rather are merely bound to a component present in serum under a variety of different conditions. Thus, careful selection of assay components to minimize or omit such molecules that can potentially bind with mutant proteins in the assay may reduce the number of false positive mutant proteins that may be inadvertently identified as positive for conditional activity. For example, in some embodiments where mutant proteins having a propensity to bind with components in human serum are being screened, bovine serum albumin may be used in the assay solution in order to reduce or eliminate the possibility of false positives caused by mutant proteins binding to components of human serum. Other similar replacements can also be made in particular cases to achieve the same goal, as will be appreciated by the skilled person.

In some embodiments, the evolving step may produce mutant proteins that also have other desired properties besides the conditionally active characteristic discussed above. Suitable other desired properties that may be evolved may include binding affinity, expression, humanization, etc. Therefore, the present invention may be employed to produce a conditionally active protein that also has an improvement in at least one or more of these other desired properties.

Depending on the parent protein used, the conditionally active protein may be an antibody or antibody fragment, a small peptide or a cyclic peptide. In some embodiments, the conditionally active protein may be further mutated using one of the mutagenesis techniques disclosed herein in, for example, by a second evolving step, to improve another property of the conditionally active protein such as binding affinity to an Fc receptor, expression, humanization, stability, etc. After the second evolving step, the mutant proteins may be screened for both the conditional activity and the improved property.

In some embodiments, the conditionally active protein of the present invention binds to ApoE3 and/or ApoE4 with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M. In some other embodiments, the conditionally active protein of the present invention binds to ApoE4 with an affinity of from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹²M, or greater than 10⁻¹² M.

In some embodiments, the conditionally active protein binds to ApoE4 present in the Golgi apparatus, but not to ApoE4 present in the endoplasmic reticulum (ER), of a cell such as a neuron or an astrocyte. In some embodiments, the conditionally active protein binds to ApoE4 present in the Golgi apparatus, but not to ApoE4 present in any other subcellular compartment or to ApoE4 present in the cytoplasm of a cell (e.g., a eukaryotic cell such as a neuron or an astrocyte).

In some embodiments, the conditionally active protein can bind to the core of an amyloid plaque, and the binding substantially co-localizes with amyloid R immunoreactivity. For example, the conditionally active protein can bind, in vitro and/or in vivo, to the ApoE4 present in an amyloid plaque, e.g., a human amyloid plaque. Whether the conditionally active protein binds to an amyloid plaque (e.g., a human amyloid plaque), can be determined using any known method. Suitable methods include an immunohistochemical method wherein the conditionally active protein is detectably labeled, either directly or indirectly.

In certain embodiments, the conditionally active proteins have a ratio of binding activity to ApoE3 and/or ApoE4 at a pH in the dementia brain to binding activity at the normal physiological pH greater than 1.0 (e.g., a high selectivity between the two conditions). The ratio of activity, or the selectivity, at the pH in the dementia brain to at the normal physiological pH may be at least about 1.3:1, or at least about 2:1, or at least about 3:1, or at least about 4:1, or at least about 5:1, or at least about 6:1, or at least about 7:1, or at least about 8:1, or at least about 9:1, or at least about 10:1, or at least about 11:1, or at least about 12:1, or at least about 13:1, or at least about 14:1, or at least about 15:1, or at least about 16:1, or at least about 17:1, or at least about 18:1, or at least about 19:1, or at least about 20:1, or at least about 30:1, or at least about 40:1, or at least about 50:1, or at least about 60:1, or at least about 70:1, or at least about 80:1, or at least about 90:1, or at least about 100:1.

In one embodiment, the conditionally active protein is an antibody, which may have a ratio of the activity or binding activity at the pH in the dementia brain to at the normal physiological pH of at least about 5:1, or at least about 6:1, or at least about 7:1, or at least about 8:1, or at least about 9:1, or at least about 10:1, or at least about 20:1, or at least about 40:1, or at least about 70:1, or at least about 100:1.

In one embodiment, the conditionally active protein is an antibody that is intended to be conjugated with another agent. The conditionally active antibody may have a high ratio of the activity or binding activity at the pH in the dementia brain to at the normal physiological pH of at least about 10:1, or at least about 11:1, or at least about 12:1, or at least about 13:1, or at least about 14:1, or at least about 15:1, or at least about 16:1, or at least about 17:1, or at least about 18:1, or at least about 19:1, or at least about 20:1, or at least about 40:1, or at least about 60:1, or at least about 80:1, or at least about 100:1.

The conditionally active protein can be employed to reduce ApoE4-amyloid β peptide (Aβ) binding. For example, the conditionally active protein can reduce ApoE4-Aβ binding by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the degree of binding between ApoE4 and AR in the absence of the conditionally active protein.

The binding between ApoE4 and amyloid β peptide may be measured by any method known in the field. In one embodiment, the binding between ApoE4 and amyloid β peptide is measured by Surface Plasmon Resonance as described in Liu et al. (“Targeting Apolipoprotein E/Amyloid R Binding by Peptoid CPO_Aβ17-21P Ameliorates Alzheimer's Disease Related Pathology and Cognitive Decline,” Sci. Rep., 2017, 7:8009).

The conditionally active protein can reduce carboxyl-terminal cleavage of ApoE4, e.g., by a neuronal cell enzyme that cleaves ApoE4 to generate neurotoxic C-terminal ApoE4 fragments. A neuronal cell enzyme that cleaves ApoE4 to generate neurotoxic C-terminal ApoE4 fragments is referred to herein as an apolipoprotein E cleavage enzyme (AECE). Neurotoxic ApoE4 fragments that are generated by action of an AECE include carboxyl-terminal truncated ApoE4, e.g., carboxyl-terminal truncated ApoE4 that include at least amino acids 244-260 of ApoE4. Neurotoxic ApoE4 fragments include carboxyl-terminal truncated ApoE4 that binds both p-tau and p-NF-H. Deletion of from about 28 to about 30, from about 30 to about 35, from about 35 to about 40, from about 40 to about 45, or from about 45 to about 48 amino acids from the carboxyl terminus of ApoE3 or ApoE4 results in carboxyl-terminal truncated ApoE that binds p-tau and p-NF-H. Specific neurotoxic carboxyl-terminal truncated ApoE4 polypeptides that give rise to neurofibrillary tangles include, but are not limited to, ApoE4Δ272-299; ApoE3Δ272-299; ApoE4Δ261-299; and ApoE4Δ252-299. See, e.g., U.S. Pat. No. 6,787,519 for a description of neurotoxic ApoE fragments.

The conditionally active protein can reduce C-terminal cleavage of ApoE4 by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99%, or more, compared to the degree of cleavage of ApoE4 by the AECE in the absence of the conditionally active protein.

ApoE4 cleavage may be measured using mass spectrometry by separating the cleavage products on an electrophoresis gel as described in Irfan et al. (“Extracellular Proteolysis of Apolipoprotein E (apoE) by Secreted Serine Neuronal Protease,” PLoS One, 2014; 9(3): e93120).

The conditionally active protein can reduce production of neurotoxic C-terminal ApoE4 fragments by an AECE in a neuron. For example, the conditionally active protein can reduce the amount of neurotoxic C-terminal ApoE4 fragments produced by action of an AECE in a neuron by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the amount of neurotoxic C-terminal ApoE4 fragments produced in the neuron in the absence of the conditionally active protein.

In some embodiments, the conditionally active protein comprises one or more non-naturally occurring amino acids. For example, the non-naturally occurring amino acid comprises a carbonyl group, an acetyl group, an aminooxy group, a hydrazine group, a hydrazide group, a semicarbazide group, an azide group, or an alkyne group. See, e.g., U.S. Pat. No. 7,632,924 for suitable non-naturally occurring amino acids. The term “non-naturally occurring amino acid” also includes amino acids produced by modification (e.g. post-translational modifications) of a naturally occurring amino acid but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

In some embodiments, the conditionally active protein is in a “mimetic” or “peptidomimetic” form, which is either entirely composed of synthetic, non-natural analogues of amino acids, or, is a chimeric molecule of partly natural occurring amino acids and partly non-natural analogs of amino acids. The mimetic can also incorporate any amount of natural occurring amino acid conservative substitutions as long as such substitutions also do not substantially alter the protein's structure and/or activity.

The mimetic form can contain any combination of non-natural structural components. In one aspect, the mimetic of the disclosure includes one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, the conditionally active protein can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., ˜C(═O)˜CH₂˜ for ˜C(═O)˜NH˜), aminomethylene (CH₂—NH), ethylene, olefin (CH═CH), ether (CH₂˜O), thioether (CH₂˜S), tetrazole, thiazole, retroamide, thioamide, or ester (see, e.g., Spatola (1983) in Chemistry and Biochemistry of Amino Acids, Peptides and Proteins, vol. 7, pp 267-357, “Peptide Backbone Modifications,” in Chemistry and Biochemistry of Amino Acids, Peptides, and Proteins, vol. 7, B. Weistein, ed., New York: Marcell Dekker, pp. 257-267).

The mimetic form may have some or all naturally occurring amino acid residues replaced by non-naturally occurring amino acid residues. The non-naturally occurring amino acid residues may be D- or L-naphylalanine; D- or L-phenylglycine; D- or L-2 thieneylalanine; D- or L-1,-2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylanines, where alkyl can be substituted or unsubstituted methyl, ethyl, propyl, hexyl, butyl, pentyl, isopropyl, iso-butyl, sec-isobutyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Acidic non-natural amino acids may be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′˜N—C—N—R′) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Basic non-natural amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivatives (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4-nitrophenol; or chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

The mimetic form of the conditionally active protein may also contain one or more amino acids of opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can also can be referred to as the R- or S-form.

The mimetic form of the conditionally active protein may be synthesized using chemical synthesis techniques. In a typical in vitro protein synthesis process, a peptide is extended in length by one amino acid by forming a peptide bond between the peptide and an amino acid. The formation of the peptide bond is carried out using a ligation reaction, which can use a natural amino acid or a non-natural amino acid. In this manner non-natural amino acids can be introduced into the polypeptides of the present invention to make mimetics.

In some embodiments, the non-naturally occurring amino acid can provide a linkage to a polymer, a second polypeptide, a scaffold, etc. In some embodiments, the conditionally active protein is linked (e.g., covalently linked) to a polymer (e.g., a polymer other than a polypeptide). Suitable polymers include, e.g., biocompatible polymers, water-soluble biocompatible polymers, synthetic polymers and naturally-occurring polymers. Examples of polymers include substituted or unsubstituted straight or branched chain polyalkylene, polyalkenylene or polyoxyalkylene polymers or branched or unbranched polysaccharides, e.g. a homo- or hetero-polysaccharide. More examples of polymers include ethylene vinyl alcohol copolymer (commonly known by the generic name EVOH or by the trade name EVAL); polybutylmethacrylate; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; poly(ethylene glycol); and carboxymethyl cellulose.

Examples of synthetic polymers include unsubstituted and substituted straight or branched chain poly(ethyleneglycol), poly(propyleneglycol) poly(vinylalcohol), and derivatives thereof, e.g., substituted poly(ethyleneglycol) such as methoxypoly(ethyleneglycol), and derivatives thereof. Suitable naturally-occurring polymers include, e.g., albumin, amylose, dextran, glycogen, and derivatives thereof.

The linked polymers can have an average molecular weight in a range of from 500 Da to 50000 Da, e.g., from 5000 Da to 40000 Da, or from 25000 to 40000 Da. For example, in some embodiments, where the conditionally active protein comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

For example, a water-soluble polymer (e.g., PEG) can be linked to the conditionally active protein by reacting the water-soluble polymer that comprises a carbonyl group to the conditionally active protein having a non-naturally occurring amino acid that comprises an aminooxy, hydrazine, hydrazide or semicarbazide group. As another example, the conditionally active protein can be linked to a water-soluble polymer by reacting the conditionally active protein that comprises an alkyne-containing amino acid with a water-soluble polymer that comprises an azide moiety. In some cases, the azide or alkyne group is linked to the PEG molecule through an amide linkage.

In some embodiments, the conditionally active protein is an scFv antibody or scFv multimer, which can be covalently linked to a PEG polymer. See, e.g., Albrecht et al. (2006) J. Immunol. Methods, 310:100. Methods and reagents suitable for linking a protein to a PEG polymer are well known in the art and may be found in, e.g., U.S. Pat. No. 5,849,860. PEG polymer suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

The PEG polymer conjugated to the conditionally active protein can be linear or branched. Branched PEG polymers include these described in U.S. Pat. No. 5,643,575, as well as “star-PEG's” and multi-armed PEG's such as those described in Shearwater Polymers, Inc. catalog “Polyethylene Glycol Derivatives 1997-1998” and U.S. Pat. No. 6,046,305.

The conditionally active protein can be glycosylated, e.g., covalently linked to a carbohydrate or polysaccharide moiety. Glycosylation of proteins is typically through N-linking or O-linking. The N-linking glycosylation refers to the attachment of the carbohydrate moiety to the side chain of an asparagine residue. The tripeptide sequences “asparagine-X-serine” or “asparagine-X-threonine,” where X is any amino acid except proline, are the recognition sequences for enzymatic attachment of the carbohydrate moiety to the asparagine side chain. Thus, the presence of either of these tripeptide sequences in a polypeptide creates a potential glycosylation site. 0-linking glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the conditionally active protein may be accomplished by altering its amino acid sequence such that it contains one or more of the above-described tripeptide sequences (for N-linking glycosylation sites). The alteration may also be made by the addition of, or substitution by, one or more serine or threonine residues to the sequence of the original antibody (for O-linking glycosylation sites). Conversely, removal of glycosylation sites can be accomplished by amino acid alteration within the native glycosylation sites of the conditionally active protein.

The conditionally active protein can be covalently linked to a second moiety (e.g., a lipid, a polypeptide, a synthetic polymer, a carbohydrate, and the like) using a linker selected from glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a reaction procedure in which the cross-linker is added to a solution containing a mixture of the polypeptides to be linked. Homobifunctional NHS ester and imido esters cross-link polypeptides containing amines. In a mild alkaline pH, imido esters react only with primary amines to form imidoamides, and overall charge of the cross-linked polypeptides is not affected. Homobifunctional sulfhydryl reactive cross-linkers include bismaleimidhexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., amine reactive moiety and a sulfhydryl-reactive moiety) and are cross-linked with one of the polypeptide and conditionally active protein via the amine or sulfhydryl reactive moiety, then reacted with the other via the non-reacted moiety. Multiple heterobifunctional haloacetyl cross-linkers are available, such as pyridyl disulfide cross-linkers. Carbodiimides are a classic example of heterobifunctional cross-linking reagents for coupling carboxyls to amines, which results in an amide bond.

In some embodiments, the conditionally active protein is linked to (e.g., covalently or non-covalently) a fusion partner, e.g., a ligand, an epitope tag, a peptide. Suitable fusion partners can improve some properties of the conditionally active protein. For example, the fusion partners can confer enhanced stability in vivo (e.g., enhanced serum half-life); provide ease of purification, e.g., (His)n, e.g., 6His, and the like; provide for secretion of the fusion protein from a cell; provide an epitope tag, e.g., GST, hemagglutinin (HA, e.g., CYPYDVPDYA, SEQ ID NO:6), FLAG (e.g., DYKDDDDK, SEQ ID NO:7), c-myc (e.g., CEQKLISEEDL, SEQ ID NO:8); provide a detectable signal, e.g., an enzyme that generates a detectable product (e.g., β-galactosidase, luciferase), or a protein that is itself detectable, e.g., a green fluorescent protein, a red fluorescent protein, a yellow fluorescent protein; provides for multimerization, e.g., a multimerization domain such as an Fc portion of an immunoglobulin; and the like.

The fusion partners may also include an affinity domain, including peptide sequences that can interact with a binding partner, e.g., such as one binding partner immobilized on a solid support useful for identification or purification. Consecutive single amino acids, such as histidine, when fused to a conditionally active protein, can be used for one-step purification of the fusion protein by high affinity binding to a resin column, such as nickel sepharose. Exemplary affinity domains as fusion partners include His5 (HHHHH) (SEQ ID NO:9), HisX6 (HHHHHH) (SEQ ID NO:10), c-myc (EQKLISEEDL) (SEQ ID NO:11), Flag (DYKDDDDK) (SEQ ID NO:12), StrepTag (WSHPQFEK) (SEQ ID NO:13), hemagglutinin, e.g., HA Tag (YPYDVPDYA; SEQ ID NO:14), glutathinone-S-transferase (GST), thioredoxin, cellulose binding domain, RYIRS (SEQ ID NO:15), FHHT (SEQ ID NO:16), chitin binding domain, S-peptide, T7 peptide, SH2 domain, C-end RNA tag, WEAAAREACCRECCARA (SEQ ID NO:17), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, parvalbumin, calbindin D9K, calbindin D28K, and calretinin, inteins, biotin, streptavidin, MyoD, leucine zipper sequences, and maltose binding protein.

In some embodiments, the conditionally active protein is modified to include a carbohydrate moiety, where the carbohydrate moiety can be covalently linked to the conditionally active protein. In some embodiments, the conditionally active protein is modified to include a lipid moiety, where the lipid moiety can be covalently linked to the conditionally active protein. Suitable lipid moieties include, e.g., an N-fatty acyl group such as N-lauroyl, N-oleoyl, etc.; a fatty amine such as dodecyl amine, oleoyl amine, etc.; a C3-C16 long-chain aliphatic lipid; and the like. See, e.g., U.S. Pat. No. 6,638,513). In some embodiments, the conditionally active protein, with or without the linked lipid moisty, is incorporated into a liposome, as described below for crossing the blood-brain barrier.

### Crossing Blood-Brain Barrier

In some therapeutic and prophylactic applications, the conditionally active protein will need to be delivered to the dementia brain where the relatively low pH will increase the binding activity of the conditionally active protein to ApoE3 and/or ApoE4. In some embodiments, the present invention also provides a mechanism to facilitate crossing of the blood-brain barrier (BBB) by the conditionally active protein.

The BBB has BBB receptor-mediated transport mechanisms that involve the vesicular trafficking system of the brain endothelium, which is known in the art as transcytosis. Briefly, a ligand of the BBB receptor in the blood circulation binds to the BBB receptor at the apical plasma membrane of the endothelial cell of the BBB. Once the ligand is bound to the receptor, the process of endocytosis is initiated as the receptor-ligand complexes cluster and membrane invagination leads to the formation of intracellular transport vesicles. The transport vesicles are subject to sorting within the cell; and in transcytosis, the vesicles containing receptor-ligand complexes or alternatively, vesicles containing dissociated ligands are sent to the basolateral side of the polarized endothelial cell, where they are released into the brain. As a result, the ligands can cross the BBB and enter the brain without disruption of the BBB.

In some embodiments, the conditionally active protein is fused to a ligand, e.g., a peptide that binds to a BBB receptor. Linking the conditionally active protein to the ligand facilitates crossing of the BBB by the conditionally active protein. Suitable peptides that bind to a BBB receptor include antibodies, e.g., monoclonal antibodies, or antigen-binding fragments thereof, as well as natural or modified ligands of a BBB receptor, that specifically bind to a BBB receptor. Suitable BBB receptors include, but are not limited to, an insulin receptor, a transferrin receptor, a leptin receptor, a lipoprotein receptor, a diphtheria toxin receptor, a heparin binding epidermal growth factor-like growth factor, and an insulin-like growth factor receptor. Some other BBB receptors include low density lipoprotein receptors such as low density lipoprotein receptor-related protein 1 and heparin-binding epidermal growth factor-like growth factor (HB-EGF). See, e.g., U.S. Patent Publication No. 2009/0156498 and Jones and Shusta, Pharm Res., vol. 24, pp. 1759-1771, 2007.

In some embodiments, the ligand fused to the conditionally active protein may be a natural ligand of one of the BBB receptors described above. The natural ligands of the BBB receptors are known in the art. In one example, the ligand may be a modified natural ligand (mutated, mimetic form) that can still bind to the BBB receptor. The natural ligands may be modified to increase stability or compatibility with the conditionally active protein.

In some embodiments, the ligand is an antibody or antibody fragment that binds to a BBB receptor. For example, the antibody or antibody fragment against an insulin receptor may be used. The antibody may be a monoclonal antibody or a chimeric antibody. The antibody or fragment binds to an exofacial epitope on the BBB receptor. The binding enables the antibody or fragment (with the conditionally active protein) to cross the BBB via a transport reaction that is mediated by the BBB receptor.

A chimeric antibody used herein as a ligand refers to a monoclonal antibody generated from a non-human animal that contains a significant human sequence such that it is not significantly immunogenic when administered to humans, e.g., at least about 80% human and up to about 20% mouse or other non-human animal, or at least about 85% human and up to about 15% mouse or other non-human animal, or at least about 90% human and up to about 10% mouse or other non-human animal, or at least about 95% human and up to about 5% mouse or other non-human animal, or greater than about 95% human and less than about 5% mouse or other non-human animal. Chimeric antibodies to a BBB receptor with sufficient human sequences for use in the invention are described in, e.g., Coloma et al., Pharm. Res., vol. 17, pp. 266-274, 2000.

The ligand of the BBB receptor may be linked to the conditionally active protein through several mechanisms. One mechanism is through a covalent linkage. For example, the linkage may be via primary amines, principally lysine residues, of either the ligand or the conditionally active protein. Another example is via chemical functionalization using Traut's reagent (2-iminothiolane) yielding a thiol that can subsequently be reacted with maleimide-functionalized ligand or conditionally active protein to form a stable thioether bond. Thiolated ligand or conditionally active protein can also be reacted with a free cysteine or reduced disulfide bond to yield a disulfide-bonded ligand-conditionally active protein conjugate. In another example, a chemical spacer (CH₂)₅NHCO(CH₂)₅NHCO or polyethylene glycol (PEG) moiety can be incorporated into the linkage to reduce steric hindrance. A PEG linkage can be used to separate the conditionally active protein and ligand, while also providing improved plasma residence time in some cases.

Another mechanism involves a non-covalent streptavidin/biotin linkage. Biotin has an extremely high binding affinity to streptavidin (Kd˜10⁻¹⁵ M). This non-covalent linkage can be used to couple a ligand to the conditionally active protein. In one example, the conditionally active protein can be monobiotinylated at lysine residues using N-hydroxysuccinimide (NHS) analogs of biotin. Alternatively, biotin can be attached to the conditionally active protein using biotin hydrazide which reacts with carboxylic acid moieties on glutamate and aspartate residues of the conditionally active protein. Having multiple choices of amino acid residues on the conditionally active protein where biotin can be attached can be helpful to ensure that the therapeutic or prophylactic activity is retained upon biotinylation. The streptavidin can be coupled to the ligand via a thioether linkage using methods described in the previous section. A BBB-crossing therapeutic or prophylactic can then be created by mixing the biotinylated conditionally active protein with the streptavidin-functionalized ligand.

Some examples of ligands of BBB receptors include: TFFYGGSRGKRNNFKTEEY (SEQ ID NO: 18, for low density lipoprotein receptor-related protein 1, RGLKLATALSLSNKFVEGS (SEQ ID NO: 19) for low-density lipoprotein receptor, THRPPMWSPVWP (SEQ ID NO: 20) for Transferrin receptor 1, and YQQILTSMPSRNVIQISNDLENLRDLLHVL (SEQ ID NO: 21) for leptin receptor.

In some embodiments, the conditionally active protein is a bi-specific conditionally active antibody with one binding site having a conditional binding activity to ApoE4 wherein the binding activity to the region is increased at a pH in a dementia brain in comparison with the binding activity to the same region of ApoE4 at a normal physiological pH. The other binding site has a conditional binding activity to a BBB receptor in which the binding activity to the BBB receptor under at least one blood plasma physiological condition is higher than the same binding activity to the BBB receptor under at least one brain physiological condition. Thus, the other binding site has a higher binding activity to the BBB receptor at the plasma side than at the brain side. A suitable method for selection of conditionally active binding activity to a BBB receptor has been described in WO 2015/175375.

Alternatively, in some embodiments, the conditionally active protein is a bi-specific conditionally active antibody with one binding site having a conditional binding activity to ApoE4 wherein the binding activity to the region is increased at a pH in a dementia brain in comparison with the binding activity to ApoE4 at a normal physiological pH, while the other binding site of the bi-specific conditionally active antibody binds to a BBB receptor but does not have conditional binding activity to a BBB receptor.

In some embodiments, the conditionally active protein may be conjugated with a polyamine. Polyamine modification may enhance permeability of the modified conditionally active protein at the BBB. The polyamine-linked conditionally active protein may passively permeate through the BBB. The conditionally active protein can be modified with polyamines that are either naturally occurring or synthetic. See, for example, U.S. Pat. No. 5,670,477. Useful naturally occurring polyamines include putrescine, spermidine, spermine, 1,3-deaminopropane, norspermidine, syn-homospermidine, thermine, thermospermine, caldopentamine, homocaldopentamine, and canavalmine. Putrescine, spermidine and spermine are particularly useful. Synthetic polyamines may have the empirical formula CₓH_{y}N_{z}, can be cyclic or acyclic, branched or unbranched, and may have hydrocarbon chains of 3-12 carbon atoms that further include 1-6 NR or N(R)₂ moieties, wherein R is H, (C₁-C₄) alkyl, phenyl, or benzyl. Polyamines can be linked to the conditionally active protein using any standard crosslinking method.

In some embodiments, the conditionally active protein is incorporated into liposomes that can fuse with the membrane of the BBB thus facilitating the crossing the BBB. Liposomes are spherical phospholipid-based nanocontainers that form spontaneously in an aqueous solution. In one example, the liposome size is controlled to be around 85 nm in diameter. The liposomes can be used to encapsulate a large amount of conditionally active protein in their aqueous core and absorb the lipophilic conditionally active protein in their lipid bilayer membrane. The liposomes may be sterically stabilized through the incorporation of PEG-distearoylphosphatidylethanolamine (DSPE) moieties into the liposome bilayer. In addition, specificity can be added to liposomes by coating their surface with a ligand to a BBB receptor.

### Conjugation

In some embodiments, the conditionally active protein may be conjugated to an agent, which may be selected from a therapeutic agent, a prophylactic agent, a diagnostic agent, a detectable label, a chelator and a contrast agent. The conditionally active protein can transport the agent to the dementia brain where the conditionally active protein is more active. In one embodiment, the agent has non-specific toxicity, which may be reduced under a normal physiological condition by being conjugated to the conditionally active protein, to thus preferentially act on the dementia brain.

In some embodiments, the conjugated agent may optionally be released from the conditionally active protein once the conditionally active protein has reached the dementia brain. In these embodiments, the conditionally active protein may act as a delivery vehicle for transporting the conjugated agents (such as therapeutic agents, prophylactic agents or diagnostic agents) to the dementia brain.

The conditionally active protein may be conjugated to the agent through covalent conjugation or non-covalent conjugation. Covalent conjugation can either be direct or via a linker. In certain embodiments, direct conjugation is by construction of a fusion protein of the agent and the conditionally active protein (i.e., by genetic fusion of the two genes encoding the conditionally active protein and the agent and expression as a single protein). In certain embodiments, direct conjugation is by formation of a covalent bond between a reactive group on the conditionally active protein and a corresponding group on the agent. In certain embodiments, direct conjugation is by modification (i.e., genetic modification) of the conditionally active protein to include a reactive group (as non-limiting examples, a sulfhydryl group or a carboxyl group) that forms a covalent attachment to the agent under appropriate conditions, or vice versa. For example, an amino acid with a desired reactive group (i.e., a cysteine residue) may be introduced into, e.g., the conditionally active protein and a disulfide bond formed with the agent. Methods for covalent conjugation of a nucleic acid to the conditionally active proteins are also known in the art (i.e., photocrosslinking, see, e.g., Zatsepin et al. Russ. Chem. Rev., 74: 77-95 (2005)).

Non-covalent conjugation can be by any non-covalent attachment means, including hydrophobic bonds, ionic bonds, electrostatic interactions, and the like, as will be readily understood by one of ordinary skill in the art.

Conjugation may also be performed using a variety of linkers. For example, a conditionally active protein and the agent may be conjugated using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine.

The linker may be a “cleavable linker” facilitating release of the neurological drug upon delivery to the brain. For example, an acid-labile linker, peptidase-sensitive linker, photolabile linker, dimethyl linker or disulfide-containing linker (Chari et al., Cancer Res., 52:127-131 (1992); U.S. Pat. No. 5,208,020) may be used. Some examples of cross-linker reagents for antibody conjugation include BMPS, EMCS, GMBS, HBVS, LC-SMCC, MBS, MPBH, SBAP, SIA, SLAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate).

The conjugated therapeutic or prophylactic agents may include magnesium compounds, anti-excitotoxic compounds (such as lubeluzole), neurotrophins, growth factors, agents that bind to beta amyloid protein with high affinity, calcium channel blockers, calcium chelators, potassium channel blockers, free radical scavengers, antioxidants, GABA agonists, GABA receptor antagonists, glutamate antagonists, NMDA antagonists, NMDA channel blockers, glycine site antagonists, polyamine site antagonists, adenosine receptor antagonists, Glial cell line derived neurotrophic factor (GDNF), brain derived neurotrophic factor, insulin like growth factor, leukocyte adhesion inhibitors, nitric oxide inhibitors, opioid antagonists, Serotonin agonists, sodium channel blockers, potassium channel openers, anti-inflammatory agents, and protein kinase inhibitors.

Specifically, the therapeutic or prophylactic agent may be calcium channel blockers such as Nimodipine, and Flunarizine; calcium chelators, such as DP-b99; potassium channel blockers; Free radical scavengers-Antioxidants such as Ebselen, porphyrin catalytic antioxidant manganese (III) meso-tetrakis (N-ethylpyridinium-2-yl) porphyrin, (MnTE-2-PyP (5+)), disodium 4-[(tert-butylimino) methyl] benzene-1,3-disulfonate N-oxide (NXY-059), N:-t-butyl-phenylnitrone or Tirilazad; GABA agonists including Clomethiazole; GABA receptor antagonists, glutamate antagonists, including AMPA antagonists such as GYKI 52466, NBQX, YM90K, YN872, ZK-200775 MPQX, Kainate antagonist SYM 2081, NMDA antagonists, including competitive NMDA antagonists such as CGS 19755 (Selfotel); NMDA channel blockers including Aptiganel (Cerestat), CP-101,606, Dextrorphan, destromethorphan, magnesium, metamine, MK-801, NPS 1506, and Remacemide; Glycine site antagonists including ACEA 1021, and GV 150026; polyamine site antagonists such as Eliprodil, and Ifenprodil; and adenosine receptor antagonists; Growth factors such as Fibroblast Growth Factor (bFGF), Glial cell line derived neurotrophic factor (GDNF), brain derived neurotrophic factor, insulin like growth factor, or neurotrophin; Leukocyte adhesion inhibitors such as Anti ICAM antibody (Enlimomab) and Hu23F2G; Nitric oxide inhibitors including Lubeluzole; opiod antagonists, such as Naloxone, Nalmefenem, Phosphatidyleholine precursor, Citicoline (CDP-coline); Serotonin agonists including Bay x 3072; Sodium channel blockers such as Fosphenytoin, Lubeluzole, and 619C89; Potassium channel openers such as BMS-204352; anti-inflamatory agents; protein kinase inhibitors, and other agents whose mechanism of action is unknown or uncertain including: Piracetam and albumin. Other active agents can provide energy to cells such as ATP, co-enzyme A, co-enzyme Q, or cytochrome C. Similarly, agents may reduce cellular demand for energy, such as phenytoin, barbital, or lithium.

In some embodiments, the conditionally active protein may be conjugated to a diagnostic agent. A diagnostic agent used in the present invention can include any diagnostic agent known in the art, as provided, for example, in the following references: Armstrong et al, Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of methods, including using the agent to provide and/or enhance a detectable signal that includes, but is not limited to, gamma-emitting, radioactive, echogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, fluorescence imaging, positron emission tomography (PET), computed tomography (CT), x-ray imaging, gamma ray imaging, and the like.

In some embodiments, the conditionally active protein can be conjugated to a chelator that binds, e.g., to metal ions to be used for a variety of diagnostic imaging techniques. Exemplary chelators include but are not limited to ethylenediaminetetraacetic acid (EDTA), [4-(1,4,8, 11-tetraazacyclotetradec-1-yl) methyljbenzoic acid (CPTA), Cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7, 10-tetraazacyclododecane-1,4,7, 10-tetra(methylene phosphonic acid) (DOTP), 1,4,8,11-tetraazacyclododecane-1,4,8, 11-tetraacetic acid (TETA), 1,4,7, 10-tetraazacyclododecane-1,4,7, 10-tetraacetic acid (DOTA), and derivatives thereof.

The conditionally active protein will, in some embodiments, be conjugated to a detectable label. Suitable detectable labels include any composition detectable by spectroscopic, photochemical, biochemical, immunochemical, electrical, optical or chemical means. Suitable detectable labels include, but are not limited to, magnetic beads (e.g. Dynabeads™), fluorescent dyes (e.g., fluorescein isothiocyanate, TEXAS RED©, rhodamine, a green fluorescent protein, a red fluorescent protein, a yellow fluorescent protein, and the like), radiolabels (e.g., ³H, ¹²⁵I, ³⁵S, ¹⁴C or ³²P), enzymes (e.g., horse radish peroxidase, alkaline phosphatase, luciferase, and others commonly used in an enzyme-linked immunosorbent assay (ELISA)), and colorimetric labels such as colloidal gold or colored glass or plastic (e.g. multistyrene, multipropylene, latex, etc.) beads.

In other embodiments, the detectable label is selected from optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook: A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents include, but are not limited to, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine (NIRD)-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine, and 3,6-diaminopyrazine-2,5-dicarboxylic acid.

In some embodiments, the conditionally active protein may be conjugated to a contrast agent, where the contrast agent is one that is suitable for use in imaging, e.g., imaging procedures carried out on humans. Non-limiting examples of contrast agent include gadolinium (Gd), dysprosium, and iron. The conditionally active protein can be labeled using standard techniques. For example, the conditionally active protein can be iodinated using chloramine T or 1,3,4,6-tetrachloro-3α,6α-dephenylglycouril. For fluorination, fluorine is conjugated to the conditionally active protein during the synthesis by a fluoride ion displacement reaction. See, Muller-Gartner, H., TIB Tech., 16:122-130 (1998) and Saji, H., Crit. Rev. Ther. Drug Carrier Syst., 16(2):209-244 (1999) for a review of synthesis of proteins with such radioisotopes. The conditionally active protein can also be labeled with a contrast agent through standard techniques. For example, the conditionally active protein can be labeled with Gd by conjugating low molecular Gd chelates such as Gd diethylene triamine pentaacetic acid (GdDTPA) or Gd tetraazacyclododecanetetraacetic (GdDOTA) to the antibody. See, Caravan et al., Chem. Rev. 99:2293-2352 (1999) and Lauffer et al., J. Magn. Reson. Imaging, 3:11-16 (1985). The conditionally active protein can be labeled with Gd by, for example, conjugating polylysine-Gd chelates to the antibody. See, for example, Curtet et al., Invest. Radiol., 33(10):752-761 (1998). Alternatively, the conditionally active protein can be labeled with Gd by incubating paramagnetic polymerized liposomes that include Gd chelator lipid with avidin and biotinylated antibody. See, for example, Sipkins et al., Nature Med., 4:623-626 (1998).

Suitable fluorescent proteins that can be conjugated to the conditionally active protein include, but are not limited to, a green fluorescent protein (GFP) from Aequoria victoria or a mutant or derivative thereof e.g., as described in U.S. Pat. Nos. 6,066,476; 6,020,192; 5,985,577; 5,976,796; 5,968,750; 5,968,738; 5,958,713; 5,919,445; 5,874,304. Examples of GFP are available commercially, e.g., from Clontech, Inc.; a red fluorescent protein; a yellow fluorescent protein; any of a variety of fluorescent and colored proteins from Anthozoan species, as described in, e.g., Matz et al. (1999) Nature Biotechnol. 17:969-973; and the like.

In yet other embodiments, the contrast agents may be x-ray contrast agents as described in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech., vol. 1, pages 183-215 (2000); Bogdanov, A. A. et al, Adv. Drug Del. Rev., Vol. 37, pages 279-293 (1999); Sachse, A. et ah, Investigative Radiology, vol. 32, pages 44-50 (1997). Examples of x-ray contrast agents include, without limitation, iopamidol, iomeprol, iohexol, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexol, iopentol, ioversol, iobitridol, iodixanol, iotrolan and iosimenol.

The conditionally active protein may in some embodiments conjugated with a “radiopaque” label, e.g. a label that can be easily visualized using for example x-rays. Radiopaque materials are well known to those of skill in the art. The most common radiopaque materials include iodide, bromide or barium salts. Other radiopaque materials are also known and include, but are not limited to organic bismuth derivatives (see, e.g., U.S. Pat. No. 5,939,045), radiopaque multiurethanes (see U.S. Pat. No. 5,346,981), organobismuth composites (see, e.g., U.S. Pat. No. 5,256,334), radiopaque barium multimer complexes (see, e.g., U.S. Pat. No. 4,866,132), and the like.

The conditionally active protein can be conjugated to a second moiety (e.g., a lipid, a polypeptide other than the conditionally active protein, a synthetic polymer, a carbohydrate, and the like) using linkers for example, glutaraldehyde, a homobifunctional cross-linker, or a heterobifunctional cross-linker Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one step reaction procedure in which the cross-linker is added to a solution containing a mixture of the polypeptides to be linked homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismaleimidhexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., amine reactive moiety and a sulfhydryl-reactive moiety) and are cross-linked with one of the polypeptides via the amine or sulfhydryl reactive moiety, then reacted with the other polypeptide via the non-reacted moiety. Multiple heterobifunctional haloacetyl cross-linkers are available, as are pyridyl disulfide cross-linkers. Carbodiimides are a classic example of heterobifunctional cross-linking reagents for coupling carboxyls to amines, which results in an amide bond.

In some embodiments, the conditionally active protein is a conditionally active antibody with the conjugation on the Fc region of the antibody. The conjugating molecules, compounds or drugs described above may be conjugated to the Fc region, as described in U.S. Pat. No. 8,362,210. For example, the Fc region may be conjugated to a therapeutic or prophylactic agent or diagnostic agent to be delivered to the dementia brain. Additional methods for conjugating to the Fc region of an antibody are known in the art. See, e.g., U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, vol. 88, pages 10535-10539, 1991; Traunecker et al., Nature, vol. 331, pages 84-86, 1988; Zheng et al., J. Immunol., vol. 154, pages 5590-5600, 1995; and Vie et al., Proc. Natl. Acad. Sci. USA, vol. 89, pages 11337-11341, 1992.

### Multispecific Conditionally Active Antibodies

In some embodiments, the conditionally active protein is a conditionally active antibody, which may be engineered to be a multispecific conditionally active antibody. The multispecific antibody may be an antibody with polyepitopic specificity, as described in WO 2013/170168. Multispecific antibodies include, but are not limited to, an antibody comprising a heavy chain variable domain (V_{H}) and a light chain variable domain (VL), where the V_{H}V_{L} unit has polyepitopic specificity, antibodies having two or more V_{L} and V_{H} domains where each V_{H}V_{L} unit binds to a different epitope, antibodies having two or more single variable domains with each single variable domain binding to a different epitope, and antibodies comprising one or more antibody fragments as well as antibodies comprising antibody fragments that have been linked covalently or non-covalently.

To construct multispecific antibodies, including bispecific antibodies, antibody fragments having at least one free sulfhydryl group are obtained. The antibody fragments may be obtained from full-length conditionally active antibodies. The conditionally active antibodies may be enzymatically digested to produce antibody fragments. Exemplary enzymatic digestion methods include, but are not limited to, pepsin, papain and Lys-C. Exemplary antibody fragments include, but are not limited to, Fab, Fab′, F(ab′)₂, Fv, diabodies (Db); tandem diabodies (taDb), linear antibodies (see U.S. Pat. No. 5,641,870, Example 2; Zapata et al., Protein Eng., vol. 8, pages 1057-1062 (1995)); one-armed antibodies, single variable domain antibodies, minibodies (Olafsen et al., Protein Eng. Design & Sel., vol. 17, pages 315-323, 2004), single-chain antibody molecules, fragments produced by a Fab expression library, anti-idiotypic (anti-Id) antibodies, CDR (complementary determining region), and epitope-binding fragments. Antibody fragments may also be produced using DNA recombinant technology. The DNA encoding the antibody fragments may be cloned into plasmid expression vectors or phagemid vectors and expressed directly in E. coli. Antibody enzymatic digestion methods, DNA cloning and recombinant protein expression methods are well known to those skilled in the art.

The conditionally active antibody may be subjected to reduction to generate a free thiol group, which can be a reactive group for reaction with a crosslinker, for example, bis-maleimide. Such a crosslinked antibody can then be reacted with a second antibody fragment having a free thiol group. The final product in which two antibody fragments are crosslinked is a multi-specific antibody. In certain embodiments, each antibody fragment is a Fab and the final product, in which the two Fabs are linked through bis-maleimide, is referred to herein as bismaleimido-(thio-Fab)₂, or bis-Fab. Such multispecific antibodies and antibody analogs, including bis-Fabs, can be used to quickly synthesize a large number of antibody fragment combinations for screening a multi-specific antibody with desired properties.

Exemplary thiol-reactive reagents include a multifunctional linker reagent that contains a capture agent, label reagent (e.g. a biotin-linker reagent), a detection label (e.g. a fluorophore reagent), a solid phase immobilization reagent (e.g. SEPHAROSE™, polystyrene, or glass), or a drug-linker intermediate. One example of a thiol-reactive reagent is N-ethyl maleimide (NEM). Such multispecific antibodies or antibody analogs having modified crosslinkers may be further reacted with a therapeutic or prophylactic agent or diagnostic agent, as described herein.

Many other techniques for making multispecific antibodies may also be used in the present invention. References describing these techniques include: (1) Milstein and Cuello, Nature, vol. 305, page 537 (1983)), WO 93/08829, and Traunecker et al., EMBO J., vol. 10, page 3655 (1991) on recombinant co-expression of two immunoglobulin heavy chain-light chain pairs having different specificities; (2) U.S. Pat. No. 5,731,168 on “knob-in-hole” engineering; (3) WO 2009/089004A1 on engineering electrostatic steering effects for making antibody Fc-heterodimeric molecules; (4) U.S. Pat. No. 4,676,980, and Brennan et al., Science, vol. 229, page 81 (1985) on cross-linking two or more antibodies or fragments; (5) Kostelny et al., J. Immunol., vol. 148, pages 1547-1553 (1992) on using leucine zippers to produce bi-specific antibodies; (6) Hollinger et al., Proc. Natl. Acad. Sci. USA, vol. 90, pages 6444-6448 (1993) on using “diabody” technology for making bispecific antibody fragments; (7) Gruber et al., J. Immunol., vol. 152, page 5368 (1994) on using single-chain Fv (sFv) dimers; (8) Tutt et al. J. Immunol. 147: 60 (1991) on preparing trispecific antibodies; and (9) US 2006/0025576A1 and Wu et al. Nature Biotechnology, vol. 25, pages 1290-1297 (2007) on engineered antibodies with three or more functional antigen binding sites, including “Octopus antibodies” or “dual-variable domain immunoglobulins” (DVDs).

Multispecific antibodies can be synthesized with modified crosslinkers such that additional functional moieties may be attached to the multispecific antibody. Modified crosslinkers allow for attachment of any sulfhydryl-reactive moiety. In one embodiment, N-succinimidyl-S-acetylthioacetate (SAT A) is attached to bis-maleimide to form bis-maleimido-acetylthioacetate (BMata). After deprotection of the masked thiol group, any functional group having a sulfhydryl-reactive (or thiol-reactive) moiety may be attached to the multispecific antibodies.

Multispecific antibodies of the present invention might also be generated using the methods as described in WO/2011/109726.

The multispecific conditionally active antibody may bind to two or more targets in the dementia brain to enhance the effectiveness for therapeutic or prophylactic treatment of neurodegenerative diseases and conditions. In one embodiment, the conditionally active antibody is engineered to be a multispecific antibody binding to both ApoE4 and amyloid β peptide or amyloid precursor protein. Multispecific antibodies may also have high selectivity at dementia brains containing all or most of the targets (antigens) that the multispecific antibody can bind to. For example, a bispecific antibody can be employed to provide selectivity for target cells by displaying a greater preference to target cells that express both of the antigens recognized by the bispecific antibody, in comparison with non-target cells that may express only one of the antigens. Therefore, due to the dynamism of the system, more bispecific antibodies are bound to the target cells than non-target cells at equilibrium.

In some embodiments, the multispecific conditionally active antibody of the present invention can bind to both ApoE4 and a BBB receptor. As discussed elsewhere in this application, binding of the multispecific conditionally active antibody to the BBB receptor may facilitate transport of the multispecific conditionally active antibody across the blood brain barrier to the brain. Once there, the multispecific conditionally active antibody can bind to ApoE4 in the brain.

### Engineering the Fc Region of Conditionally Active Antibodies

The conditionally active antibodies of the present invention may be engineered at their Fc region. The Fc region is the tail region of an antibody that interacts with Fc receptors and some proteins of the complement system. Unlike the Fab region that is specific for each antigen, the Fc region of all antibodies in a class is the same for each species regardless which antigen the antibody binds.

The Fc receptors are members of the immunoglobulin gene superfamily of proteins. Fc receptors are found on a number of cells in the immune system including phagocytes like macrophages and monocytes, granulocytes like neutrophils and eosinophils, and lymphocytes of the innate immune system (natural killer cells) or adaptive immune system (e.g., B cells). After binding with an antibody, the Fc receptor activates these cells and allows these cells to identify and eliminate antigens (such as amyloid R) that are bound on the Fab region of the antibody. Fc receptor mediated killing mechanisms include complement-dependent cytotoxicity (CDC), antibody-dependent cellular cytotoxicity (ADCC), and antibody-dependent cellular phagocytosis (ADCP).

In some embodiments, the Fc region of the conditionally active antibody is engineered to introduce mutations such as amino acid substitutions in the Fc region. Such substitutions may increase the half-life of the mutated antibody in serum. For example, the half-life of an IgG antibody is correlated with its pH-dependent binding to the neonatal receptor FcRn, which is expressed on the surface of endothelial cells and protects the IgG in a pH-dependent manner from degradation. Several amino acid substitutions at the Fc region, such as T250Q/M428L and M252Y/S254T/T256E+H433K/N434F, have shown increased binding affinity of the antibody to FcRn and extend the half-life of the antibody.

Amino acid substitutions may also be introduced to the Fc region to alter effector functions. For example, human antibodies in the IgG class bind to Fcγ receptors (FcγRI, FcγRIIa, FcγRIIIa), the inhibitory FcγRIIb receptor, and the first component of complement (C1q) with different affinities, yielding very different effector functions among different antibodies. Binding of IgG antibody to FcγRs or C1q depends on residues located in the hinge domain and the CH2 domain of the antibody. Amino acid substitutions in human antibodies IgG1 or IgG2 residues at positions 233-236 and antibody IgG4 residues at positions 327, 330 and 331 can greatly reduce ADCC and CDC. Furthermore, alanine substitution at different positions in the Fc region, including K322, can significantly reduce complement activation. Many more examples of engineering the Fc region are described in U.S. Pat. No. 8,362,210.

In some embodiments, the Fc region of an antibody may be engineered to be capable of recognizing an antigen as described, for example, in US 2010/0256340. At least one, preferably two, extra Fab fragments may be linked to the Fc region of the antibody. In some embodiments, the extra Fab fragment(s) are conditionally active. For example, a conditionally active antibody of the present invention designed for crossing the BBB may contain an extra Fab fragment with affinity for a BBB receptor on the plasma side and little or no affinity for a BBB receptor on the brain side.

The conditionally active protein may be modified through a natural process or using a chemical modification technique, as described in WO 2016/138071. The conditionally active protein may be synthesized using a solid-phase chemical peptide synthesis method, also as described in WO 2016/138071.

### Composition, Formulation, Kit

The conditionally active proteins of the present invention may be included in pharmaceutical compositions, medical devices, kits, or articles of manufacture for therapeutic, prophylactic or diagnostic use, particularly in humans. Suitable pharmaceutical compositions, medical devices, kits, or articles of manufacture are described in WO 2016/138071.

In some embodiments, the pharmaceutical composition may be in a liquid form, a lyophilized form or a liquid form reconstituted from a lyophilized form. The lyophilized preparation is typically reconstituted with a sterile solution prior to administration. The standard procedure for reconstituting a lyophilized composition is to add a volume of pure water (typically about equivalent to the volume removed during lyophilization). Solutions comprising antibacterial agents may also be used for the production of pharmaceutical compositions for parenteral administration; see also Chen, Drug Dev Ind Pharm, vol. 18, pp. 1311-54, 1994.

Exemplary concentrations of the conditionally active protein in a pharmaceutical composition may be in a range of from about 1 mg/mL to about 200 mg/ml, or from about 50 mg/mL to about 200 mg/mL, or from about 150 mg/mL to about 200 mg/mL.

An aqueous formulation of the conditionally active protein may be prepared in a pH-buffered solution, e.g., at a solution pH ranging from about 4.0 to about 7.0, or from about 5.0 to about 6.0, or alternatively about 5.5. Examples of buffers that are suitable for providing a pH within this range include phosphate-, histidine-, citrate-, succinate-, and acetate-buffers, as well as other known organic acid buffers. The buffer concentration can be from about 1 mM to about 100 mM, or from about 5 mM to about 50 mM, depending, e.g., on the buffer and the desired tonicity of the formulation.

A tonicity agent may be included in the composition to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions may also be used. The term “isotonic” denotes a solution having the same tonicity as some other solution with which it is compared, such as physiological salt solution or serum. Tonicity agents may be used in an amount of about 5 mM to about 350 mM, or in an amount of 100 mM to 350 nM.

A pharmaceutically acceptable surfactant may also be added to the composition to reduce aggregation of the formulated conditionally active protein and/or minimize the formation of particulates in the formulation and/or reduce adsorption. Exemplary surfactants include polyoxyethylensorbitan fatty acid esters, polyoxyethylene alkyl ethers, alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Exemplary concentrations of surfactant may range from about 0.001% to about 1% w/v.

A lyoprotectant may also be added to the composition in order to protect the labile active ingredient (e.g. a protein) against destabilizing conditions during the lyophilization process. For example, known lyoprotectants include sugars (including glucose and sucrose), polyols (including mannitol, sorbitol and glycerol), and amino acids (including alanine, glycine and glutamic acid). Lyoprotectants can be included in an amount of about 10 mM to 500 nM.

In some embodiments, the composition, containing one or more of a surfactant, a buffer, a stabilizer, and a tonicity agent, is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative selected from ethanol, benzyl alcohol, phenol, m-cresol, p-chlor-m-cresol, methyl or propyl parabens, benzalkonium chloride, and combinations thereof, may be included in the formulation, e.g., at concentrations ranging from about 0.001 to about 2% (w/v).

In one embodiment, an exemplary composition containing the conditionally active protein is a liquid or reconstituted lyophilized formulation suitable for parenteral administration, and can comprise: about 1 mg/mL to about 200 mg/mL of the conditionally active protein; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

Another exemplary composition is a parenteral formulation in a liquid or reconstituted lyophilized form, comprising: about 1 mg/mL to about 200 mg/mL of the conditionally active protein; 0.04% Tween 20 w/v; 20 mM L-histidine; and 250 mM Sucrose; and has a pH of 5.5.

Another exemplary composition is also a parenteral formulation comprising: 1) 15 mg/mL of the conditionally active protein; 0.04% Tween 20 w/v; 20 mM L-histidine; and 250 mM sucrose; and has a pH of 5.5; or 2) 75 mg/mL of the conditionally active protein; 0.04% Tween 20 w/v; 20 mM L-histidine; and 250 mM sucrose; and has a pH of 5.5; or 3) 75 mg/mL of the conditionally active protein; 0.02% Tween 20 w/v; 20 mM L-histidine; and 250 mM Sucrose; and has a pH of 5.5; or 4) 75 mg/mL of the conditionally active protein; 0.04% Tween 20 w/v; 20 mM L-histidine; and 250 mM trehalose; and has a pH of 5.5; or 5) 75 mg/mL of the conditionally active protein; 0.02% Tween 20 w/v; 20 mM L-histidine; and 250 mM trehalose; and has a pH of 5.5.

Yet other exemplary compositions are parenteral formulations 1-12 in a liquid form, comprising: 1) 7.5 mg/mL of the conditionally active protein; 0.022% Tween 20 w/v; 120 mM L-histidine; and 250 125 mM sucrose; and has a pH of 5.5; or 2) 37.5 mg/mL of the conditionally active protein; 0.02% Tween 20 w/v; 10 mM L-histidine; and 125 mM sucrose; and has a pH of 5.5; or 3) 37.5 mg/mL of the conditionally active protein; 0.01% Tween 20 w/v; 10 mM L-histidine; and 125 mM sucrose; and has a pH of 5.5; or 4) 37.5 mg/mL of the conditionally active protein; 0.02% Tween 20 w/v; 10 mM L-histidine; 125 mM trehalose; and has a pH of 5.5; or 5) 37.5 mg/mL of the conditionally active protein; 0.01% Tween 20 w/v; 10 mM L-histidine; and 125 mM trehalose; and has a pH of 5.5; or 6) 5 mg/mL of the conditionally active protein; 0.02% Tween 20 w/v; 20 mM L-histidine; and 250 mM trehalose; and has a pH of 5.5; or 7) 75 mg/mL of the conditionally active protein; 0.02% Tween 20 w/v; 20 mM L-histidine; and 250 mM mannitol; and has a pH of 5.5; or 8) 75 mg/mL of the conditionally active protein; 0.02% Tween 20 w/v; 20 mM L histidine; and 140 mM sodium chloride; and has a pH of 5.5; or 9) 150 mg/mL of the conditionally active protein; 0.02% Tween 20 w/v; 20 mM L-histidine; and 250 mM trehalose; and has a pH of 5.5; or 10) 150 mg/mL of the conditionally active protein; 0.02% Tween 20 w/v; 20 mM L-histidine; and 250 mM mannitol; and has a pH of 5.5; or 11) 150 mg/mL of the conditionally active protein; 0.02% Tween 20 w/v; 20 mM L-histidine; and 140 mM sodium chloride; and has a pH of 5.5; or 12) 10 mg/mL of the conditionally active protein; 0.01% Tween 20 w/v; 20 mM L-histidine; and 40 mM sodium chloride; and has a pH of 5.5.

In some embodiments, the conditionally active protein can be utilized in aerosol formulation to be administered via inhalation. The conditionally active protein can be formulated into pressurized acceptable propellants such as dichlorodifluoromethane, propane, nitrogen and the like.

Unit dosage forms for oral administration such as syrups, elixirs, and suspensions may be provided wherein each dosage unit, for example, teaspoonful, tablespoonful, tablet or vile, contains a predetermined amount of the composition. Similarly, unit dosage forms for injection or intravenous administration may comprise the conditionally active protein in a composition as a solution in sterile water, normal saline or another pharmaceutically acceptable carrier.

The conditionally active protein may be formulated as an injectable formulation. Typically, injectable compositions are prepared as liquid solutions or suspensions; solid forms suitable for solution in, or suspension in, liquid vehicles prior to injection may also be prepared. The preparation may also be emulsified or the conditionally active protein encapsulated in liposome vehicles.

In some embodiments, the conditionally active protein may be formulated as aerosol and intranasal compositions. For suppositories, the composition will include traditional binders and carriers such as, polyalkylene glycols, or triglycerides. Such compositions may be formed from mixtures containing the conditionally active protein in the range of about 0.5% to about 10% (w/w), e.g., about 1% to about 2%.

The conditionally active protein may be formulated as intranasal formulations including vehicles that neither cause significant irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the conditionally active proteins by the nasal mucosa.

In some embodiments, the conditionally active protein is formulated in a controlled release formulation. Controlled release within the scope of this invention means one of a number of extended release dosage forms. The following types of controlled release may be used for the purposes of the present invention: continuous release, delayed release, gradual release, long-term release, programmed release, prolonged release, proportionate release, protracted release, slow release, spaced release, sustained release, timed release, delayed action, extended action, layered-time action, long acting, prolonged action, repeated action, sustained action, and extended release. Further discussions of these terms and methods for making the same may be found in Lesczek Krowczynski, Extended-Release Dosage Forms, 1987 (CRC Press, Inc.).

Controlled release composition may be prepared using methods known in the art. Examples of controlled-release preparations include semipermeable matrices of solid hydrophobic polymers containing the antibody in which the matrices are in the form of shaped articles, e.g. films or microcapsules. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(−)-3-hydroxybutyric acid. Possible loss of biological activity and possible changes in immunogenicity of the conditionally active protein comprised in sustained-release formulation may be reduced or prevented by using appropriate additives, by controlling moisture content and by developing specific polymer matrix compositions.

Controlled release technologies include both physical systems and chemical systems. Physical systems include reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

There are a number of controlled release drug formulations for oral administration. These controlled release formulations include osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.).

The conditionally active protein may be administered to a patient/subject using any available method and route suitable for drug delivery, including in vivo and ex vivo methods, as well as systemic and localized routes of administration. Conventional and pharmaceutically acceptable routes of administration include intranasal, intramuscular, intratracheal, subcutaneous, intradermal, topical application, intravenous, intraarterial, rectal, nasal, oral, and other enteral and parenteral routes of administration. Routes of administration may be combined, if desired, or adjusted depending upon the conditionally active protein and/or the desired effect. The conditionally active protein can be administered in a single dose or in multiple doses. In some embodiments, the conditionally active protein is administered orally. In some embodiments, the conditionally active protein is administered via an inhalational route. In some embodiments, the conditionally active protein is administered intranasally. In some embodiments, the conditionally active protein is administered locally. In some embodiments, the conditionally active protein is administered intracranially. In some embodiments, the conditionally active protein is administered intravenously.

In some embodiments, the conditionally active protein is administered by injection and/or delivery, e.g., to a site in a brain artery or directly into brain tissue. The conditionally active protein can also be administered directly to a target site (e.g., a brain region containing amyloid plaques), e.g., by biolistic delivery to the target site.

In some embodiments, the conditionally active protein is administered by intrathecal administration for direct introduction into the central nervous system (CNS). One method of intrathecal delivery is intracerebroventricular (ICV) administration such as by injection, infusion and/or an ICV implantable port to deliver the conditionally active protein into the cerebrospinal fluid (CSF). The conditionally active protein of the present invention may also be delivered by intrathecal lumbar injection and/or infusion for direct introduction into the CSF.

In some embodiments, the conditionally active protein is provided in a combination therapy with one or more additional therapeutic or prophylactic agents. Suitable additional therapeutic or prophylactic agents include, but are not limited to, acetylcholinesterase inhibitors, including, but not limited to, Aricept (donepezil), Exelon (rivastigmine), metrifonate, and tacrine (Cognex); non-steroidal anti-inflammatory agents, including, but not limited to, ibuprofen and indomethacin; cyclooxygenase-2 (Cox2) inhibitors such as Celebrex; and monoamine oxidase inhibitors, such as Selegilene (Eldepryl or Deprenyl). Dosages for each of the above agents are known in the art. For example, Aricept is generally administered at 50 mg orally per day for 6 weeks, and, if well tolerated by the individual, at 10 mg per day thereafter. For example, the combination therapy comprises administration of effective amounts of the conditionally active protein and a drug that inhibits apoE4 domain interaction (e.g., an agent as described in U.S. Patent Publication No. 2006/0073104; and in Ye et al., Proc. Natl. Acad. Sci. USA, vol. 102, pp. 18700, 2005).

### Methods of Treatment

The present invention also includes a method of treating a neurodegenerative disease comprising administrating the isolated polypeptide, the conditionally active protein or the conjugated conditionally active protein of to a subject with a neurodegenerative disease.

The neurodegenerative disease is selected from Parkinson's disease (PD), Alzheimer's disease (AD), Huntington's disease (HD), age-related macular degeneration (AMD), retinitis pigmentosa (RP), amyotrophic lateral sclerosis (ALS, e.g., familial ALS and sporadic ALS), multiple system atrophy, progressive supranuclear palsy, down syndrome, diffuse Lewy body disease, multiple sclerosis (MS), and brain trauma.

Any of the isolated polypeptides, conditionally active proteins or conjugated conditionally active proteins provided herein may be used in therapeutic methods. In one aspect, these proteins are used as a medicament. In further aspects, the isolated polypeptides, conditionally active proteins or conjugated conditionally active proteins are for use in treating neurodegenerative diseases. In one such embodiment, the method further comprises administering to the individual an effective amount of at least one additional therapeutic agent.

It is understood that any of the above formulations or therapeutic methods may be carried out using the isolated polypeptides, conditionally active proteins or conjugated conditionally active proteins.

The following examples are illustrative, but not limiting, of the soft gelatin capsules of the present disclosure. Other suitable modifications and adaptations of the variety of conditions and parameters normally encountered in the field, and which are obvious to those skilled in the art, are within the scope of the disclosure.

### EXAMPLES

### Examples 1-12

Examples 1-12 for making conditionally active proteins are described in WO 2017/078839.

### Example 13

Apolipoprotein E (ApoE) acts as a major cholesterol carrier supporting lipid transport and tissue repair in the brain as well as mediating clearance of amyloid-beta peptides. ApoE proteins bind to several cell surface receptors including low-density lipoprotein receptor (LDLR) and very low-density lipoprotein receptor (VLDLR) to deliver lipids. ApoE also binds to Sortilin which mediates uptake of ApoE containing lipoproteins into neurons, and to amyloid-beta (Aβ) peptide, which are thought to play important roles in the pathogenesis of Alzheimer Disease (AD).

The ApoE gene is the main genetic determinant of AD risk in humans. See Alzheimer's Risk Gene ApoE4 and Autism, AustismWeb2 (2020). The ApoE gene is polymorphic, having three prominent alleles, ApoE2, ApoE3, and ApoE4, with ApoE3 being the most common allele. ApoE4 carriers are significantly more likely to develop AD, while ApoE2 carriers have a moderately reduced risk of AD.

Naturally occurring physiological chemicals (such as bicarbonate and sodium hydrogen sulfide) modulate the binding activities of antibodies as a function of the differential external cellular pH between the acidic tumor microenvironment (pH 5.8-6.7 resulting from glycolysis) and the alkaline environment of blood and normal tissues (pH 7.4 and higher) and is referred to as Protein-associated Chemical Switches or PaCS. See Chang H W, Frey G, Liu H, Xing C, Steinman L, Boyle W J, Short J M, Proc. Natl. Acad. Sci. USA, 2021, March 2, 118(9). This discovery enabled the use of these physiological PaCS chemicals to increase the therapeutic index of cancer therapies. Since both senescent and other inflamed cells associated with AD are also glycolytic, leading to acidic external microenvironments, we investigated the effects of PaCS molecules on the binding activities of different ApoE isoforms to amyloid-b (Aβ) peptide and cell surface receptors as a function of pH.

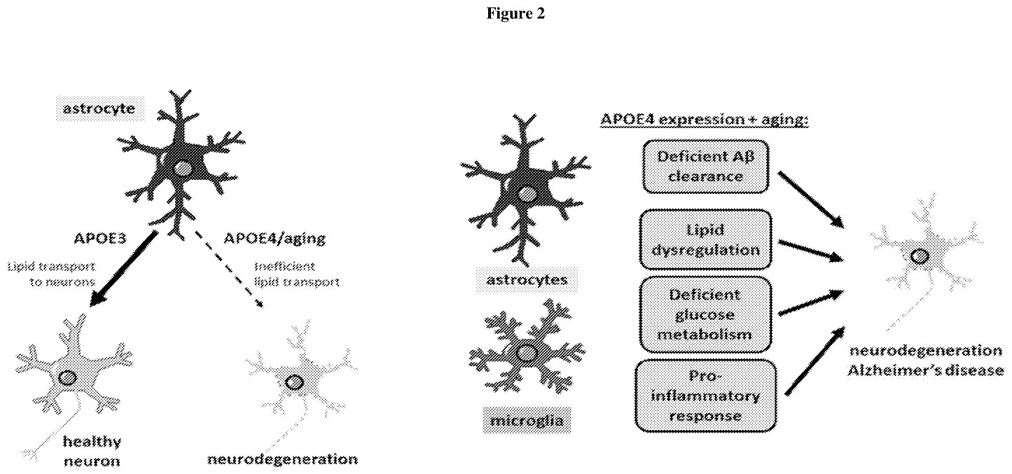

FIG. 2 shows ApoE3 expression vs ApoE4 expression A) Astrocytes expressing ApoE4 have inefficient lipid transport, sensitizing neurons to degeneration. B) ApoE4 expression disrupts multiple homeostatic pathways in astrocytes and microglia to cause neurodegeneration and AD. See “The Role of APOe4 in Disrupting the Homeostatic Functions of Astrocytes and Microglia in Aging and Alzheimer's Disease,” Celia G. Fernandez et al., (2019), Frontiers in Aging Neuroscience, 11(14).

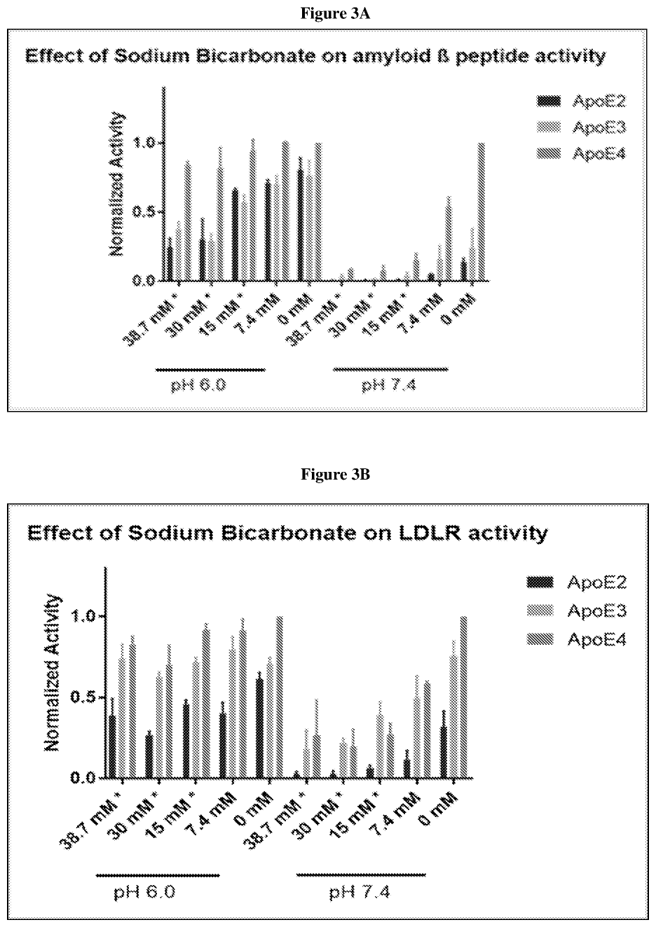

FIGS. 3A-3D show that the binding of ApoE Isoforms to amyloid beta peptides, LDLR, VLDLR and Sortilin at pH 6.0 and pH 7.4 is concentration dependent, based on the concentration of the PaCS molecule, sodium bicarbonate.

FIGS. 3A-3D show the results of pH affinity ELISA assays performed using amyloid beta peptides (Biolegend) (A), LDLR (B), VLDLR (C), Sortilin (D) as coating antigens (B,C,D Sino Biological). ApoE isoforms (Sigma Aldrich) were diluted in PBS+/−sodium bicarbonate. Binding of the ApoE isoforms to the antigens were detected with anti-ApoE antibody (Biolegend) and anti-mouse IgG conjugated with HRP (Promega). The bicarbonate physiological range is marked with an asterisk. Data were normalized to ApoE4 values.
ApoE4 showed the highest binding activities at both acidic and alkaline conditions compared to other ApoE isoforms.
Sodium bicarbonate affected the binding of ApoE isoforms to amyloid beta peptides, LDLR, VLDLR and Sortilin in a concentration dependent manner.
Considerably reduced binding activities were observed under alkaline conditions vs acidic conditions in the presence of sodium bicarbonate for all ApoE isoforms.

TABLE 1
Influence of sodium bicarbonate on the pH selectivity of ApoE Isoforms
binding to amyloid beta peptides, LDLR, VLDLR and Sortilin

| | | | | | | Fold change - | |
|---|---|---|---|---|---|---|---|
| | | | EC50-pH 6.0, | EC50-pH 7.4, | Fold change - | PBS + sodium | |
| ApoE | EC50-pH 6.0, | EC50-pH 7.4, | PBS + sodium | PBS + sodium | PBS (pH | bicarb (pH | |
| isoforms | PBS (ug/ml) | PBS (ug/ml) | bicarb (ug/ml) | bicarb (ug/ml) | 6.0/pH 7.4) | 6.0/pH 7.4) | |
| ApoE2 | 1.48 | 5.43 | 4.64 | 26.72 | 3.67 | 5.75 | Amyloid β |
| ApoE3 | 2.19 | 9.05 | 4.68 | 28.64 | 4.13 | 6.12 | peptides |
| ApoE4 | 0.35 | 2.53 | 1.22 | 13.36 | 7.23 | 10.91 | |
| ApoE2 | 2.14 | 7.33 | 5.98 | 16.68 | 3.43 | 2.79 | LDLR |
| ApoE3 | 2.30 | 4.75 | 2.04 | 6.03 | 2.06 | 2.96 | |
| ApoE4 | 0.60 | 3.05 | 1.52 | 6.56 | 5.11 | 4.32 | |
| ApoE2 | 1.33 | 4.64 | 2.49 | 10.63 | 3.49 | 4.26 | VLDLR |
| ApoE3 | 2.34 | 6.79 | 2.45 | 17.24 | 2.90 | 7.05 | |
| ApoE4 | 0.41 | 1.08 | 0.48 | 3.10 | 2.65 | 6.48 | |
| ApoE2 | 1.77 | 4.71 | 4.15 | 19.70 | 2.66 | 4.75 | Sortilin |
| ApoE3 | 2.06 | 7.84 | 4.45 | 19.02 | 3.80 | 4.27 | |
| ApoE4 | 0.38 | 2.10 | 1.36 | 7.38 | 5.55 | 5.45 | |

### Observations

PaCS molecules generally reduced the binding of all ApoE isoforms against all four protein targets in both acidic and alkaline microenvironments, with the greatest reduction of binding occurring under alkaline conditions. The binding activities of ApoE4 to amyloid beta peptides, LDLR and Sortilin had the highest fold change in binding between pH 6.0 and 7.4, as compared to ApoE2 and ApoE3 in the presence of sodium bicarbonate. ApoE2 showed a greater decrease in binding activity to LDLR in alkaline conditions in the presence of sodium bicarbonate, as compared to ApoE3 and ApoE4. The binding activity of ApoE3 to LDLR was not affected by the pence of sodium bicarbonate in acidic conditions in contrast to the binding activities of ApoE2 and ApoE4.

Table 1: pH affinity ELISA assay was performed using amyloid beta peptides, LDLR, VLDLR and Sortilin as coating antigens. ApoE isoforms were serially diluted in PBS without sodium bicarbonate (PBS) or in PBS with 30 mM sodium bicarbonate (PBS+sodium bicarb) at pH 6.0 or pH 7.4. Binding of the ApoE isoforms to the antigens wsd detected with anti-ApoE antibody and anti-mouse IgG conjugated with HRP. EC50 values were calculated using the nonlinear fit model (variable slope, four parameters) built into GraphPad Prism software version 7.03. EC50 (ug/mL) values were averaged from two representative experiments. Fold change were calculated using EC50 values of pH 7.4 divided by EC50 values of pH 6.0.

Table 2 for Examples 14-16

| Clones in FIGS. 4A-4B | SEQ ID NO. |
|---|---|
| BAP191-VH-WT | SEQ ID NO: 22 |
| BAP191-01-VH-M034D | SEQ ID NO: 23 |
| BAP191-01-VH-I051D | SEQ ID NO: 24 |
| BAP191-01-VH-R098E | SEQ ID NO: 25 |
| BAP191-01-VH-M099E | SEQ ID NO: 26 |
| BAP191-01-VH-M104E | SEQ ID NO: 27 |
| BAP191-VK-WT or BAP191-VL-WT | SEQ ID NO: 28 |
| BAP191-VK-Y091D or BAP191-VL-Y091D | SEQ ID NO: 29 |
| BAP191-VK-P095D or BAP191-VL-P095D | SEQ ID NO: 30 |

### Example 14: BAP191.01 Anti-ApoE pH Affinity ELISA with hApoE3 Antigen

### 1.1 Test Articles

Anti-ApoE wildtype antibody (BAP191.01-WT, benchmark)
Anti-ApoE CAB mutations at various site indicated in Table 3 below:

TABLE 3

| CAB mutations | Site | VL Region | VH Region |
|---|---|---|---|
| BAP191.01-CPE- | heavy chain, amino | SEQ ID NO: 28 | SEQ ID NO: 27 |
| HC-M104E | acid 104 | (BAP191-VK- | (BAP191-01-VH- |
| | | WT) | M104E) |
| BAP191.01-CPE- | light chain, amino | SEQ ID NO: 30 | SEQ ID NO: 22 |
| LC-P095D | acid 95 | (BAP191-VK- | (BAP191-VH-WT) |
| | | P095D) | |

### 1.2 Formulations

Test articles were first diluted to 5000 ng/mL in pH 6.0 or pH 7.4 ELISA (enzyme-linked immunosorbent assay) incubation buffer. Then 5000 ng/mL of test articles were 5-fold serially diluted in pH 6.0 or pH 7.4 ELISA incubation buffer.

### 1.3 pH Affinity ELISA Assay

1) Coated ELISA plates with 100 μL of 2 μg/mL recombinant huApoE3 (human Apolipoprotein E3) antigen in PBS (phosphate buffered saline) buffer.
2) Covered plates with sealing film and incubate overnight at 4° C.
3) Decanted plates and tap out residual liquid on a stack of paper towels.
4) Washed wells twice by dispensing 200 μL of pH 6.0 or pH 7.4 ELISA incubation buffer to the wells and completely aspirated the contents.
5) Added 200 μL of pH 6.0 or pH 7.4 ELISA incubation buffer to the wells. Covered with sealing film and placed the plate onto a plate shaker set to 50 rpm (revolutions per minute) for 60 minutes at room temperature.
6) Decanted plates and tapped out residual liquid on a stack of paper towels.
7) Serially diluted test articles in 5-fold dilutions starting at 5000 ng/mL in pH 6.0 or pH 7.4 ELISA incubation buffer.
8) Added 100 μL/well of diluted test articles to the plates
9) Covered with sealing film and place the plates onto a plate shaker set to 50 rpm for 60 minutes at room temperature.
10) Decanted plates and tap out residual liquid on a stack of paper towels.
11) Washed wells three times by dispensing 200 μL of pH 6.0 or pH 7.4 ELISA wash buffer to the wells and completely aspirated the contents.
12) Diluted the HRP (horseradish peroxidase) secondary antibody at 1:2500 in pH 6.0 or pH 7.4 ELISA incubation buffer.
13) Added 100 μL HRP secondary antibody diluted in pH 6.0 or pH 7.4 ELISA incubation buffer to each well.
14) Covered with sealing film and placed the plates onto a plate shaker set to 50 rpm for 60 minutes at room temperature.
15) Decanted plates and tap out residual liquid on a stack of paper towels.
16) Washed wells three times by dispensing 200 μL of pH 6.0 or pH 7.4 ELISA wash buffer to the wells and completely aspirated the contents.
17) Dispensed 50 μL per well of the TMB (3,3′,5,5′ tetramethylbenzidine) substrate solution into all wells of the plates. Incubated at room temperature for about 10 minutes.
18) Added 50 μL per well of 1N HCl into all wells of the plates. Read plates at 450 nm using PerkinElmer, EnSpire 2300 Multilabel Reader.

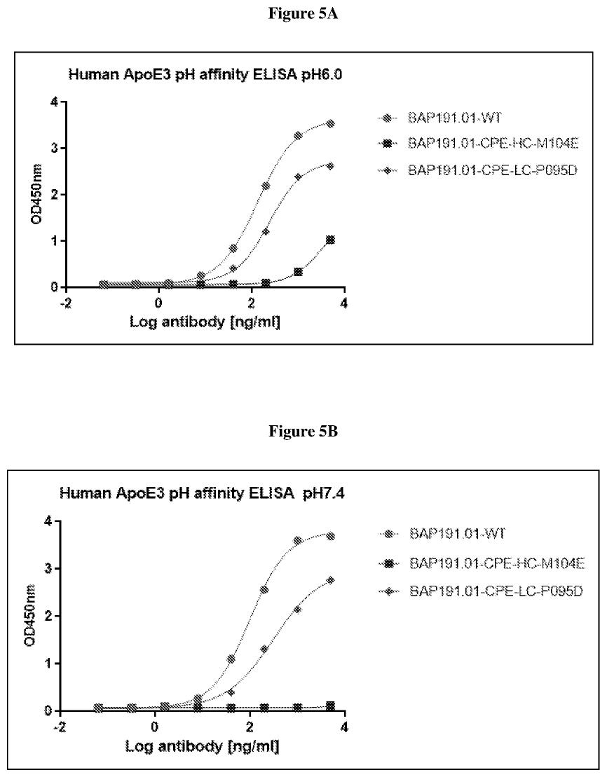

TABLE 4
Results as shown in FIGS. 5A and 5B
EC50 (ng/mL)

| | hApoE3- | hApoE3- | Ratio pH |
|---|---|---|---|
| sample | pH 6.0 | pH 7.4 | 7.4/6.0 |
| BAP191.01-WT | 136.1 | 99.09 | 0.73 |
| BAP191.01-CPE-HC-M104E | 3135 | 9253 | 2.95 |
| BAP191.01-CPE-LC-P095D | 244.9 | 310.9 | 1.27 |

### Example 15: BAP191.01 Anti-ApoE pH Affinity ELISA with hApoE4 Antigen

### 1.1 Test Articles

Anti-ApoE wildtype antibody (BAP191.01-WT, benchmark)
Anti-ApoE CAB mutations at various site indicated in the Table 5 below:

TABLE 5

| CAB mutations | Site | VL Region | VH Region |
|---|---|---|---|
| BAP191.01-CPE-HC- | heavy chain, | SEQ ID NO: 28 | SEQ ID NO: 27 |
| M104E | amino acid 104 | (BAP191-VK-WT) | (BAP191-01- |
| | | | VH-M104E) |
| BAP191.01-CPE-LC- | light chain, | SEQ ID NO: 30 | SEQ ID NO: 22 |
| P095D | amino acid 95 | (BAP191-VK- | (BAP191-VH- |
| | | P095D) | WT) |
| BAP191-01-CPE-HC- | heavy chain, | SEQ ID NO: 28 | SEQ ID NO: 23 |
| M034D | amino acid 34 | (BAP191-VK-WT) | (BAP191-01- |
| | | | VH-M034D) |
| BAP191.01-CPE-HC- | heavy chain, | SEQ ID NO: 28 | SEQ ID NO: 24 |
| I051D | amino acid 51 | (BAP191-VK-WT) | (BAP191-01- |
| | | | VH-1051D) |
| BAP191.01-CPE-HC- | heavy chain, | SEQ ID NO: 28 | SEQ ID NO: 25 |
| R098E | amino acid 98 | (BAP191-VK-WT) | (BAP191-01- |
| | | | VH-R098E) |
| BAP191.01-CPE-HC- | heavy chain, | SEQ ID NO: 28 | SEQ ID NO: 26 |
| M099E | amino acid 99 | (BAP191-VK-WT) | (BAP191-01- |
| | | | VH-M099E) |
| BAP191.01-CPE-LC- | light chain, | SEQ ID NO: 29 | SEQ ID NO: 22 |
| Y091D | amino acid 91 | (BAP191-VK- | (BAP191-VH- |
| | | Y091D) | WT) |

### 1.2 Formulations

Test articles were first diluted to 5000 ng/mL in pH 6.0 or pH 7.4 ELISA incubation buffer. Then 5000 ng/mL of test articles were 5-fold serially diluted in pH 6.0 or pH 7.4 ELISA incubation buffer.

### 1.3 pH Affinity ELISA Assay

1) Coated ELISA plates with 100 μL of 2 μg/mL recombinant huApoE3 (human Apolipoprotein E3) antigen in PBS (phosphate buffered saline) buffer.
2) Covered plates with sealing film and incubate overnight at 4° C.
3) Decanted plates and tap out residual liquid on a stack of paper towels.
4) Washed wells twice by dispensing 200 μL of pH 6.0 or pH 7.4 ELISA incubation buffer to the wells and completely aspirated the contents.
5) Added 200 μL of pH 6.0 or pH 7.4 ELISA incubation buffer to the wells. Covered with sealing film and placed the plate onto a plate shaker set to 50 rpm (revolutions per minute) for 60 minutes at room temperature.
6) Decanted plates and tapped out residual liquid on a stack of paper towels.
7) Serially diluted test articles in 5-fold dilutions starting at 5000 ng/mL in pH 6.0 or pH 7.4 ELISA incubation buffer.
8) Added 100 μL/well of diluted test articles to the plates
9) Covered with sealing film and place the plates onto a plate shaker set to 50 rpm for 60 minutes at room temperature.
10) Decanted plates and tap out residual liquid on a stack of paper towels.
11) Washed wells three times by dispensing 200 μL of pH 6.0 or pH 7.4 ELISA wash buffer to the wells and completely aspirated the contents.
12) Diluted the HRP (horseradish peroxidase) secondary antibody at 1:2500 in pH 6.0 or pH 7.4 ELISA incubation buffer.
13) Added 100 μL HRP secondary antibody diluted in pH 6.0 or pH 7.4 ELISA incubation buffer to each well.
14) Covered with sealing film and placed the plates onto a plate shaker set to 50 rpm for 60 minutes at room temperature.
15) Decanted plates and tap out residual liquid on a stack of paper towels.
16) Washed wells three times by dispensing 200 μL of pH 6.0 or pH 7.4 ELISA wash buffer to the wells and completely aspirated the contents.
17) Dispensed 50 μL per well of the TMB (3,3′,5,5′ tetramethylbenzidine) substrate solution into all wells of the plates. Incubated at room temperature for about 10 minutes.
18) Added 50 μL per well of 1N HCl into all wells of the plates. Read plates at 450 nm using PerkinElmer, EnSpire 2300 Multilabel Reader.

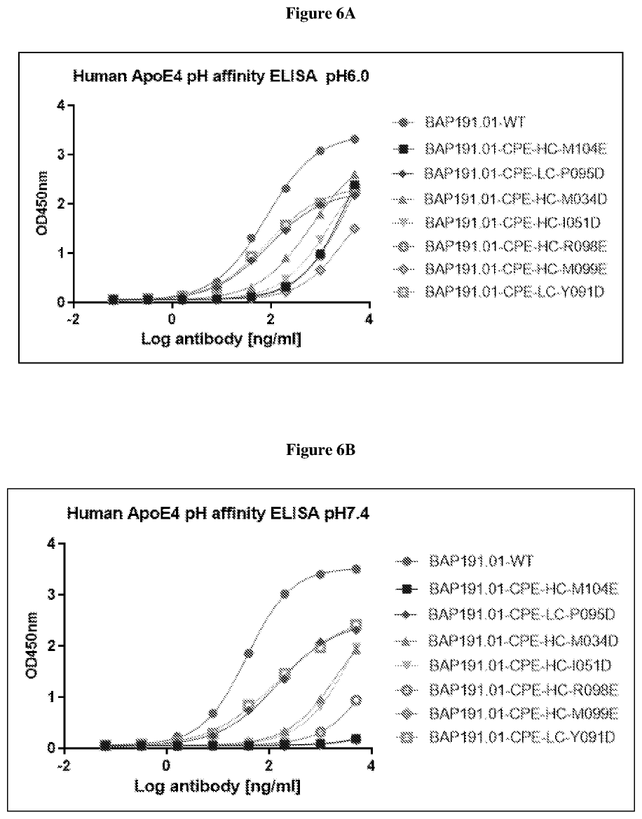

TABLE 6
Results as shown in FIGS. 6A and 6B
EC50 (ng/mL)

| | hApoE4- | hApoE4- | Ratio pH |
|---|---|---|---|
| sample | pH 6.0 | pH 7.4 | 7.4/6.0 |
| BAP191.01-WT | 79.72 | 36.77 | 0.46 |
| BAP191.01-CPE-HC-M104E | 4199 | 6.87E+07 | 18383.42 |
| BAP191.01-CPE-LC-P095D | 89.26 | 147 | 1.65 |
| BAP191-01-CPE-HC-M034D | 684.6 | 2058 | 3.01 |
| BAP191.01-CPE-HC-1051D | 1544 | 4746 | 3.07 |
| BAP191.01-CPE-HC-R098E | 4360 | 18859 | 4.33 |
| BAP191.01-CPE-HC-M099E | 2928 | 6.89E+06 | 2351.78 |
| BAP191.01-CPE-LC-Y091D | 83.98 | 150.1 | 1.79 |

### Example 16: BAP191.01 Anti-ApoE pH Affinity ELISA with ms ApoE Antigen

### 1.1 Test Articles

Anti-ApoE wildtype antibody (BAP191.01-WT, benchmark)
Anti-ApoE CAB mutations at various site indicated in the Table 7 below:

TABLE 7

| CAB mutations | Site | VL Region | VH Region |
|---|---|---|---|
| BAP191.01-CPE-HC- | heavy chain, | SEQ ID NO: 28 | SEQ ID NO: 27 |
| M104E | amino acid 104 | (BAP191-VK- | (BAP191-01-VH- |
| | | WT) | M104E) |
| BAP191.01-CPE-LC- | light chain, | SEQ ID NO: 30 | SEQ ID NO: 22 |
| P095D | amino acid 95 | (BAP191-VK- | (BAP191-VH- |
| | | P095D) | WT) |
| BAP191-01-CPE-HC- | heavy chain, | SEQ ID NO: 28 | SEQ ID NO: 23 |
| M034D | amino acid 34 | (BAP191-VK- | (BAP191-01-VH- |
| | | WT) | M034D) |
| BAP191.01-CPE-HC- | heavy chain, | SEQ ID NO: 28 | SEQ ID NO: 24 |
| I051D | amino acid 51 | (BAP191-VK- | (BAP191-01-VH- |
| | | WT) | I051D) |
| BAP191.01-CPE-HC- | heavy chain, | SEQ ID NO: 28 | SEQ ID NO: 25 |
| R098E | amino acid 98 | (BAP191-VK- | (BAP191-01-VH- |
| | | WT) | R098E) |
| BAP191.01-CPE-HC- | heavy chain, | SEQ ID NO: 28 | SEQ ID NO: 26 |
| M099E | amino acid 99 | (BAP191-VK- | (BAP191-01-VH- |
| | | WT) | M099E) |

### 1.2 Formulations

Test articles were first diluted to 5000 ng/mL in pH 6.0 or pH 7.4 ELISA incubation buffer. Then 5000 ng/mL of test articles were 5-fold serially diluted in pH 6.0 or pH 7.4 ELISA incubation buffer.

### 1.3 pH Affinity ELISA Assay

1) Coated ELISA plates with 100 μL of 2 μg/mL recombinant mouse ApoE3 antigen in PBS (phosphate buffered saline) buffer.
2) Covered plates with sealing film and incubated overnight at 4° C.
3) Decanted plates and tap out residual liquid on a stack of paper towels.
4) Washed wells twice by dispensing 200 μL of pH 6.0 or pH 7.4 ELISA incubation buffer to the wells and completely aspirated the contents.
5) Added 200 μL of pH 6.0 or pH 7.4 ELISA incubation buffer to the wells. Covered with sealing film and placed the plate onto a plate shaker set to 50 rpm (revolutions per minute) for 60 minutes at room temperature.
6) Decanted plates and tapped out residual liquid on a stack of paper towels.
7) Serially diluted test articles in 5-fold dilutions starting at 5000 ng/mL in pH 6.0 or pH 7.4 ELISA incubation buffer.
8) Added 100 μL/well of diluted test articles to the plates
9) Covered with sealing film and place the plates onto a plate shaker set to 50 rpm for 60 minutes at room temperature.
10) Decanted plates and tap out residual liquid on a stack of paper towels.
11) Washed wells three times by dispensing 200 μL of pH 6.0 or pH 7.4 ELISA wash buffer to the wells and completely aspirated the contents.
12) Diluted the HRP (horseradish peroxidase) secondary antibody at 1:2500 in pH 6.0 or pH 7.4 ELISA incubation buffer.
13) Added 100 μL HRP secondary antibody diluted in pH 6.0 or pH 7.4 ELISA incubation buffer to each well.
14) Covered with sealing film and placed the plates onto a plate shaker set to 50 rpm for 60 minutes at room temperature.
15) Decanted plates and tap out residual liquid on a stack of paper towels.
16) Washed wells three times by dispensing 200 μL of pH 6.0 or pH 7.4 ELISA wash buffer to the wells and completely aspirated the contents.
17) Dispensed 50 μL per well of the TMB (3,3′,5,5′ tetramethylbenzidine) substrate solution into all wells of the plates. Incubated at room temperature for about 10 minutes.
18) Added 50 μL per well of 1N HCl into all wells of the plates. Read plates at 450 nm using PerkinElmer, EnSpire 2300 Multilabel Reader.

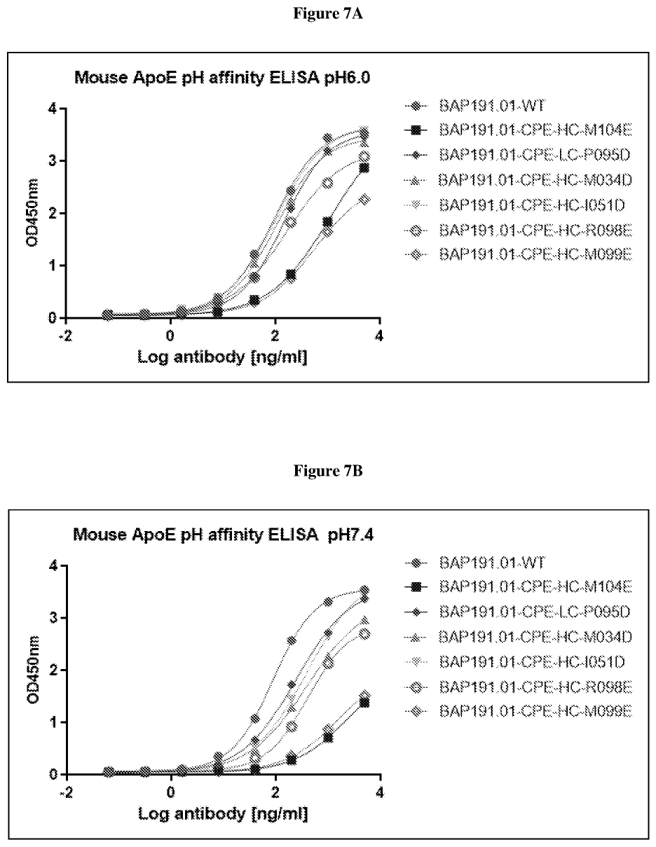

TABLE 8
Results as shown in FIGS. 7A and 7B
EC50 (ng/mL)

| | msApoE- | msApoE- | Ratio pH |
|---|---|---|---|
| sample | pH 6.0 | pH 7.4 | 7.4/6.0 |
| BAP191.01-WT | 92.6 | 88.96 | 0.96 |
| BAP191.01-CPE-HC-M104E | 1090 | 2454 | 2.25 |
| BAP191.01-CPE-LC-P095D | 146,5 | 244.1 | 1.67 |
| BAP191-01-CPE-HC-M034D | 104.8 | 389.3 | 3.71 |
| BAP191.01-CPE-HC-1051D | 103.6 | 389.9 | 3.76 |
| BAP191.01-CPE-HC-R098E | 163.2 | 413.1 | 2.53 |
| BAP191.01-CPE-HC-M099E | 572.5 | 1577 | 2.75 |

### CONCLUSIONS

Multiple factors are implicated for the manifestation of AD, including viral infection, chronic inflammation, senescent cells, poor protein recycling and other underlying factors that are associated with the build-up of amyloid containing plaques. Astrocytes, for example, which play an important role in amyloid beta clearance, can initiate senescence in response to certain stressors. Astrosenescence leads to the production of proinflammatory factors know as senescence-associated secretory phenotype (SASP) which are involved in the initiation and progression of AD and are known to be glycolytic.

As shown in Example 13, the effects of PaCS molecules on the binding activities of different ApoE isoforms to amyloid-β (Aβ) peptide and cell surface receptors as a function of pH were investigated. At physiological concentrations, bicarbonate and sodium hydrogen sulfide (data not shown) were observed to differentially affect the binding activities of the ApoE isoforms to LDLR, VLDLR, Sortilin and Aβ peptide. Large reductions in binding of ApoE under alkaline conditions versus acidic conditions in the presence of PaCS molecules were observed, with ApoE4 showing the highest binding activity, especially under acidic conditions relative to the other isoforms. The data indicates that PaCS molecules can conditionally (pH dependent) modulate the interactions of the different ApoE isoforms with various cell surface receptors. These results provide potential mechanistic insights into the differential activity of the ApoE isoforms in normal, alkaline blood versus acidic microenvironments associated with inflammation, as well as insights into the potential for isoform-dependent protection from PaCS molecules in the pathogenesis of neurodegenerative diseases.

As demonstrated in Example 14, anti-ApoE CAB mutants showed active binding to huApoE3 at a lower pH of 6.0 (FIG. 5A) but decreased binding affinity or virtually inactive binding to huApoE3 at pH 7.4 in comparison to the parent BAP191.01 WT (FIG. 5B).

As demonstrated in Example 15, anti-ApoE CAB mutants showed active binding to huApoE4 at a lower pH of 6.0 (FIG. 6A) but decreased binding affinity or virtually inactive binding to huApoE4 at pH 7.4 in comparison to the parent BAP191.01 WT (FIG. 6B).

Similarly. In Example 16, anti-ApoE CAB mutants showed active binding to msApoE3 at a lower pH of 6.0 (FIG. 7A) but decreased binding to msApoE3 at pH 7.4 in comparison to the parent BAP191.01-WT (FIG. 7B).

These data suggest that PaCS-dependent Conditionally Active Biologic or CAB therapies that target proteins or cells in these glycolytic, acidic microenvironments such as that in the brain of subjects with a neurodegenerative disease or disorder, may lead to safer and more potent therapies that might also be administered at earlier stages of AD progression for improved outcomes.

All documents mentioned herein are hereby incorporated by reference in their entirety and at least to provide the disclosure for which they were specifically relied upon or cited as referring to. The applicant(s) do not intend to dedicate any disclosed embodiments to the public, and to the extent any disclosed modifications or alterations may not literally fall within the scope of the claims, they are considered to be part hereof under the doctrine of equivalents

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meanings of the terms in which the appended claims are expressed.

1. An isolated polypeptide comprising:
a heavy chain variable region having three complementarity determining regions having H1, H2, and H3 amino acid sequences, wherein:
(a) the H1 amino acid sequence is GYTFTTAGX₁Q (SEQ ID NO: 31),
(b) the H2 amino acid sequence is WX₂NTHSGEPKYAEDFKG (SEQ ID NO: 32), and
(c) the H3 amino acid sequence is X₃GGYAX₄DY (SEQ ID NO: 33);
wherein X₁ is M or D,
X₂ is I or D,
X₃ is M or E, and
X₄ is M or E; and
a light chain variable region having three complementarity determining regions having L1, L2, and L3 amino acid sequences, wherein:
(d) the L1 amino acid sequence is KASEDINSYLS (SEQ ID NO: 34),
(e) the L2 amino acid sequence is RANRLVD (SEQ ID NO: 35), and
(f) the L3 amino acid sequence is LQX₅DEFX₆LT (SEQ ID NO: 36);
wherein X₅ is Y or D and X₆ is P or D;
with the proviso that X₁, X₂, X₃, X₄, X₅ and X₆ cannot be M, I, M, M, Y, P, respectively, at the same time.

2. The polypeptide of claim 1, wherein the H1 sequence is selected from the group consisting of SEQ ID NOs: 37 and 38, the H2 sequence is selected from the group consisting of SEQ ID NOs: 39 and 40, and the H3 sequence is selected from the group consisting of SEQ ID NOs: 44-46.

3. The polypeptide of claim 1, wherein the heavy chain variable region is selected from the group consisting of:
the heavy chain variable region having the H1 sequence of SEQ ID NO: 37, the H2 sequence is SEQ ID NO: 39, and the H3 sequence is SEQ ID NO: 44;
the heavy chain variable region having the H1 sequence of SEQ ID NO: 37, the H2 sequence is SEQ ID NO: 40, and the H3 sequence is SEQ ID NO: 44;
the heavy chain variable region having the H1 sequence of SEQ ID NO: 38, the H2 sequence is SEQ ID NO: 39, and the H3 sequence is SEQ ID NO: 44;
the heavy chain variable region having the H1 sequence of SEQ ID NO: 37, the H2 sequence is SEQ ID NO: 39, and the H3 sequence is SEQ ID NO: 45; and
the heavy chain variable region having the H1 sequence of SEQ ID NO: 37, the H2 sequence is SEQ ID NO: 39, and the H3 sequence is SEQ ID NO: 46.

4. The polypeptide of claim 1, wherein the heavy chain variable region has an amino acid sequence selected from the amino acid sequences of SEQ ID NOs: 22-27 and the light chain variable region has an amino acid sequence selected from the amino acid sequences of SEQ ID NOs: 28-30.

5. (canceled)

6. The polypeptide of claim 2, wherein the L1 sequence is SEQ ID NO: 34, the L2 sequence is SEQ ID NO: 35, and the L3 sequence is selected from the group consisting of SEQ ID NOs: 41-43.

7. The polypeptide of claim 3, wherein the L1 sequence is SEQ ID NO: 34, the L2 sequence is SEQ ID NO: 35, and the L3 sequence is selected from the group consisting of SEQ ID NOs: 42-43.

8. (canceled)

9. The polypeptide of claim 1, wherein the heavy chain variable region has the H1 sequence of SEQ ID NO: 37, the H2 sequence of SEQ ID NO: 39, and the H3 sequence of SEQ ID NO: 44; and
the light chain variable region has the L1 sequence of SEQ ID NO: 34, the L2 sequence of SEQ ID NO: 35, and the L3 sequence selected from the group consisting of SEQ ID NOs: 42-43.

10. The polypeptide of claim 1, wherein the heavy chain variable region has an amino acid sequence of SEQ ID NO: 22; and.
the light chain variable region has an amino acid sequence selected from the amino acid sequences of SEQ ID NOs: 29-30.

11. The polypeptide of claim 1, wherein the heavy chain variable region has an amino acid sequence selected from the group consisting of SEQ ID NOs: 23-27; and.
the light chain variable region has an amino acid sequence of SEQ ID NO: 28.

12. The polypeptide of claim 1, wherein the polypeptide is selected from the group consisting of:
a polypeptide wherein the heavy chain variable region has an amino acid sequence of SEQ ID NO: 23 and the light chain variable region has an amino acid sequence of SEQ ID NO: 28;
a polypeptide wherein the heavy chain variable region has an amino acid sequence of SEQ ID NO: 24 and the light chain variable region has an amino acid sequence of SEQ ID NO: 28;
a polypeptide wherein the heavy chain variable region has an amino acid sequence of SEQ ID NO: 25 and the light chain variable region has an amino acid sequence of SEQ ID NO: 28;
a polypeptide wherein the heavy chain variable region has an amino acid sequence of SEQ ID NO: 26 and the light chain variable region has an amino acid sequence of SEQ ID NO: 28;
a polypeptide wherein the heavy chain variable region has an amino acid sequence of SEQ ID NO: 27 and the light chain variable region has an amino acid sequence of SEQ ID NO: 28;
a polypeptide wherein the heavy chain variable region has an amino acid sequence of SEQ ID NO: 22 and the light chain variable region has an amino acid sequence of SEQ ID NO: 29; and
a polypeptide wherein the heavy chain variable region has an amino acid sequence of SEQ ID NO: 22 and the light chain variable region has an amino acid sequence of SEQ ID NO:
30.

13. A conditionally active protein comprising the polypeptide of claim 1.

14. The conditionally active protein of claim 13, wherein the conditionally active protein binds to ApoE4.

15. The conditionally active protein of claim 13, wherein the conditionally active protein binds to ApoE with an increased binding activity at an aberrant condition in comparison to the binding activity of the conditionally active protein at a normal physiological condition and the conditionally active protein binds to ApoE with a decreased binding activity at a normal physiological pH in comparison to a conditionally active protein having a heavy chain variable region of SEQ ID NO: 22 and a light chain variable region of SEQ ID NO: 28.

16. The conditionally active protein of claim 13, wherein the conditionally active protein is evolved from a parent protein and the binding activity of the conditionally active protein to ApoE at the normal physiological condition is less than the binding activity of the parent protein at ApoE at the normal physiological condition.

17. The conditionally active protein of claim 15, wherein the aberrant condition is a pH in a range of from about 5.0 to about 7.0, or from about 5.4 to about 6.8, or from about 5.8 to about 6.8, or from about 6.0 to about 6.8, or from about 6.6 to about 6.8.

18. The conditionally active protein of claim 17, wherein the normal physiological condition is a pH is in a range of from about 7.2 to about 7.8, or from about 7.2 to about 7.6, or from about 7.2 to about 7.4.

19. The conditionally active protein of claim 17, wherein the conditionally active protein binds to ApoE at the aberrant pH with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

20. The conditionally active protein of 13, wherein the conditionally active protein has a ratio of the binding activity to ApoE at the pH of the dementia brain to the binding activity to ApoE at the normal physiological pH of at least about 2:1, or at least about 5:1, or at least about 10:1, or at least about 20:1, or at least about 50:1, or at least about 100:1.

21. The conditionally active protein of claim 20, wherein the pH of the dementia brain is in a range of from about 5.0 to about 7.0, or from about 5.4 to about 6.8, or from about 5.8 to about 6.8, or from about 6.0 to about 6.8, or from about 6.6 to about 6.8. and the normal physiological pH is in a range of from about 7.2 to about 7.8, or from about 7.2 to about 7.6, or from about 7.2 to about 7.4.

22. The conditionally active protein of claim 21, wherein the conditionally active protein binds to ApoE at the pH of the dementia brain with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M.

23. The conditionally active protein of claim 13, wherein a therapeutically or prophylactically effective amount of the conditionally active protein reduces ApoE4-amyloid β peptide binding by at least about 10%, at least about 20%, at least about 50%, at least about 90%, compared to the binding between ApoE4 and amyloid β peptide in the absence of the conditionally active protein.

24. The conditionally active protein of claim 13, wherein a therapeutically or prophylactically effective amount of the conditionally active protein reduces C-terminal cleavage of ApoE4 by at least about 5%, at least about 10%, at least about 20%, at least about 50%, at least about 90%, at least about 95%, at least about 99%, compared to the cleavage of ApoE4 in the absence of the conditionally active protein.

25. The conditionally active protein of claim 13, wherein the conditionally active protein binds to amyloid plaques.

26. The conditionally active protein of claim 13, wherein the conditionally active protein comprises at least one non-naturally occurring amino acid.

27. The conditionally active protein of claim 13, wherein the conditionally active protein is glycosylated.

28. The conditionally active protein of claim 13, wherein the conditionally active protein is selected from the group consisting of an antibody, an antigen binding antibody fragment and a multi-specific antibody capable of binding to a receptor on the blood-brain barrier and a binding activity of the multi-specific antibody to the receptor on the blood-brain barrier under a blood plasma physiological condition is higher than the same binding activity to the receptor on the blood brain barrier under at least one brain physiological condition.

29. The conditionally active protein of any one of claims 13-27, wherein the conditionally active protein is selected from the group consisting of a small peptide having at most 25 amino acid residues, and a cyclic peptide.

30-32. (canceled)

33. The conditionally active protein of claim 28, wherein the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

34. A conjugated conditionally active protein comprising the conditionally active protein of claim 13 conjugated to a ligand of a receptor on the blood-brain barrier, a polyamine, a therapeutic agent, a prophylactic agent, a diagnostic agent, a detectable label, a chelator or a contrast agent.

35. The conjugated conditionally active protein of claim 34, wherein the conditionally active protein is conjugated to the ligand and the ligand is selected from the group consisting of an antibody of the receptor on the blood-brain barrier, a natural ligand of the receptor on the blood-brain barrier, and a modified ligand derived from a natural ligand of the receptor on the blood-brain barrier.

36. (canceled)

37. The conjugated conditionally active protein of claim 34, wherein the conditionally active protein is conjugated to the ligand and the ligand is selected from a peptide having an amino acid sequence of SEQ ID NO: 18, 19, 20, or 21.

38. The conjugated conditionally active protein of claim 34, wherein the conditionally active protein is conjugated to the ligand and the receptor on the blood-brain barrier is selected from at least one of an insulin receptor, a transferrin receptor, a leptin receptor, a lipoprotein receptor, a diphtheria toxin receptor, a heparin binding epidermal growth factor-like growth factor, and an insulin-like growth factor receptor, a low density lipoprotein receptor-related protein 1, low density lipoprotein receptor-related protein 1, and heparin-binding epidermal growth factor-like growth factor.

39. The conjugated conditionally active protein of claim 34, wherein the conditionally active protein is conjugated to the polyamine.

40. The conjugated conditionally active protein of claim 34, wherein the conditionally active protein is conjugated to the therapeutic agent or the prophylactic agent.

41. The conjugated conditionally active protein of claim 34, wherein the therapeutic or prophylactic agent is selected from at least one of magnesium compounds, anti-excitotoxic compounds, growth factors, agents that bind to beta amyloid protein, calcium channel blockers, calcium chelators, potassium channel blockers, free radical scavengers, antioxidants, GABA agonists, GABA receptor antagonists, glutamate antagonists, NMDA antagonists, NMDA channel blockers, glycine site antagonists, polyamine site antagonists, adenosine receptor antagonists, leukocyte adhesion inhibitors, nitric oxide inhibitors, opioid antagonists, Serotonin agonists, sodium channel blockers, potassium channel openers, anti-inflammatory agents, and protein kinase inhibitors.

42. The conjugated conditionally active protein of claim 41, wherein the therapeutic or prophylactic agent is the growth factor and the growth factor is selected from a Glial cell line derived neurotrophic factor, a brain derived neurotrophic factor, an insulin like growth factor, a fibroblast growth factor, and a neurotrophin.

43. The conjugated conditionally active protein of claim 41, wherein the therapeutic or prophylactic agent is the calcium channel blocker and the calcium channel blocker is selected from nimodipine and flunarizine.

44. The conjugated conditionally active protein of claim 34, wherein the conditionally active protein is conjugated to a diagnostic agent.

45. A diagnostic agent comprising the conditionally active protein of claim 13 and a detectable label, a chelator or a contrast agent.

46-49. (canceled)

50. A method of generating, from a parent protein having a known binding activity to ApoE at a normal physiological pH, a conditionally active protein for prevention or treatment of a neurodegenerative disease, comprising steps of:
a) mutating the parent protein to generate a set of mutant proteins;
b) subjecting the set of mutant proteins to a first assay for binding activity to ApoE at a pH of a dementia brain and a second assay for binding activity to ApoE at a normal physiological pH; and
c) selecting the conditionally active protein from the mutant proteins of step b) that have an increased binding activity to ApoE in the first assay in comparison to the binding activity to ApoE in the second assay and which has a decreased binding activity to ApoE at a normal physiological pH in comparison to the parent protein.

51-60. (canceled)

61. A method of treatment of a neurodegenerative disease comprising a step of administering the isolated polypeptide of claim 1 to a subject suffering from the neurodegenerative disease.

62. (canceled)

63. The use of method of claim 61, wherein the neurodegenerative disease is selected from Parkinson's disease (PD), Alzheimer's disease (AD), Huntington's disease (HD), age-related macular degeneration (AMD), retinitis pigmentosa (RP), amyotrophic lateral sclerosis (ALS, e.g., familial ALS and sporadic ALS), multiple system atrophy, progressive supranuclear palsy, down syndrome, diffuse Lewy body disease, multiple sclerosis (MS), and brain trauma.


SEQUENCE LISTING (46 sequences)

SEQ ID NO: 1 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 2 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 3 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 4 - protein, 5 aa, Homo sapiens
ASHLR

SEQ ID NO: 5 - protein, 4 aa, Homo sapiens
SDNH

SEQ ID NO: 6 - protein, 10 aa, synthetic construct
  REGION 1..10: note = Synthetic construct
CYPYDVPDYA

SEQ ID NO: 7 - protein, 8 aa, synthetic construct
  REGION 1..8: note = synthetic construct
DYKDDDDK

SEQ ID NO: 8 - protein, 11 aa, synthetic construct
  REGION 1..11: note = synthetic construct
CEQKLISEEDL

SEQ ID NO: 9 - protein, 5 aa, synthetic construct
  REGION 1..5: note = synthetic construct
HHHHH

SEQ ID NO: 10 - protein, 6 aa, synthetic construct
  REGION 1..6: note = synthetic construct
HHHHHH

SEQ ID NO: 11 - protein, 10 aa, synthetic construct
  REGION 1..10: note = synthetic construct
EQKLISEEDL

SEQ ID NO: 12 - protein, 8 aa, synthetic construct
  REGION 1..8: note = synthetic construct
DYKDDDDK

SEQ ID NO: 13 - protein, 8 aa, synthetic construct
  REGION 1..8: note = synthetic construct
WSHPQFEK

SEQ ID NO: 14 - protein, 9 aa, synthetic construct
  REGION 1..9: note = synthetic construct
YPYDVPDYA

SEQ ID NO: 15 - protein, 5 aa, synthetic construct
  REGION 1..5: note = synthetic construct
RYIRS

SEQ ID NO: 16 - protein, 4 aa, synthetic construct
  REGION 1..4: note = synthetic construct
FHHT

SEQ ID NO: 17 - protein, 17 aa, synthetic construct
  REGION 1..17: note = synthetic construct
WEAAAREACCRECCARA

SEQ ID NO: 18 - protein, 19 aa, synthetic construct
  REGION 1..19: note = synthetic construct
TFFYGGSRGKRNNFKTEEY

SEQ ID NO: 19 - protein, 19 aa, synthetic construct
  REGION 1..19: note = synthetic construct
RGLKLATALSLSNKFVEGS

SEQ ID NO: 20 - protein, 12 aa, synthetic construct
  REGION 1..12: note = synthetic construct
THRPPMWSPVWP

SEQ ID NO: 21 - protein, 30 aa, synthetic construct
  REGION 1..30: note = synthetic construct
YQQILTSMPSRNVIQISNDLENLRDLLHVL

SEQ ID NO: 22 - protein, 117 aa, synthetic construct
  REGION 1..117: note = synthetic construct
QIQLVQSGPELKKPGETVKISCKASGYTFTTAGMQWVQKMPGKGFKWIGWINTHSGEPKYAEDFKGRFAFSLETSASTAYLQISNLKNEDTATYFCARMGGYAMDYWGQGTSVTVSS

SEQ ID NO: 23 - protein, 117 aa, synthetic construct
  REGION 1..117: note = synthetic construct
QIQLVQSGPELKKPGETVKISCKASGYTFTTAGDQWVQKMPGKGFKWIGWINTHSGEPKYAEDFKGRFAFSLETSASTAYLQISNLKNEDTATYFCARMGGYAMDYWGQGTSVTVSS

SEQ ID NO: 24 - protein, 117 aa, synthetic construct
  REGION 1..117: note = synthetic construct
QIQLVQSGPELKKPGETVKISCKASGYTFTTAGMQWVQKMPGKGFKWIGWDNTHSGEPKYAEDFKGRFAFSLETSASTAYLQISNLKNEDTATYFCARMGGYAMDYWGQGTSVTVSS

SEQ ID NO: 25 - protein, 117 aa, synthetic construct
  REGION 1..117: note = synthetic construct
QIQLVQSGPELKKPGETVKISCKASGYTFTTAGMQWVQKMPGKGFKWIGWINTHSGEPKYAEDFKGRFAFSLETSASTAYLQISNLKNEDTATYFCAEMGGYAMDYWGQGTSVTVSS

SEQ ID NO: 26 - protein, 117 aa, synthetic construct
  REGION 1..117: note = synthetic construct
QIQLVQSGPELKKPGETVKISCKASGYTFTTAGMQWVQKMPGKGFKWIGWINTHSGEPKYAEDFKGRFAFSLETSASTAYLQISNLKNEDTATYFCAREGGYAMDYWGQGTSVTVSS

SEQ ID NO: 27 - protein, 117 aa, synthetic construct
  REGION 1..117: note = synthetic construct
QIQLVQSGPELKKPGETVKISCKASGYTFTTAGMQWVQKMPGKGFKWIGWINTHSGEPKYAEDFKGRFAFSLETSASTAYLQISNLKNEDTATYFCARMGGYAEDYWGQGTSVTVSS

SEQ ID NO: 28 - protein, 107 aa, synthetic construct
  REGION 1..107: note = synthetic construct
DIKLTQSPSSMYASLGERVTITCKASEDINSYLSWFQQKPGKSPKTLIYRANRLVDGVPSRFSGSGSGQDYSLTISSLEYEDMGIYYCLQYDEFPLTFGAGTKLELT

SEQ ID NO: 29 - protein, 107 aa, synthetic construct
  REGION 1..107: note = synthetic construct
DIKLTQSPSSMYASLGERVTITCKASEDINSYLSWFQQKPGKSPKTLIYRANRLVDGVPSRFSGSGSGQDYSLTISSLEYEDMGIYYCLQDDEFPLTFGAGTKLELT

SEQ ID NO: 30 - protein, 107 aa, synthetic construct
  REGION 1..107: note = synthetic construct
DIKLTQSPSSMYASLGERVTITCKASEDINSYLSWFQQKPGKSPKTLIYRANRLVDGVPSRFSGSGSGQDYSLTISSLEYEDMGIYYCLQYDEFDLTFGAGTKLELT

SEQ ID NO: 31 - protein, 10 aa, synthetic construct
  REGION 1..10: note = synthetic construct
  VARIANT 9: note = Xaa is M or D
GYTFTTAGXQ

SEQ ID NO: 32 - protein, 17 aa, synthetic construct
  REGION 1..17: note = synthetic construct
  VARIANT 2: note = Xaa is I or D
WXNTHSGEPKYAEDFKG

SEQ ID NO: 33 - protein, 8 aa, synthetic construct
  REGION 1..8: note = synthetic construct
  VARIANT 1: note = Xaa is M or E
  VARIANT 6: note = Xaa is M or E
XGGYAXDY

SEQ ID NO: 34 - protein, 11 aa, synthetic construct
  REGION 1..11: note = synthetic construct
KASEDINSYLS

SEQ ID NO: 35 - protein, 7 aa, synthetic construct
  REGION 1..7: note = synthetic construct
RANRLVD

SEQ ID NO: 36 - protein, 9 aa, synthetic construct
  REGION 1..9: note = synthetic construct
  VARIANT 3: note = Xaa is Y or D
  VARIANT 7: note = Xaa is P or D
LQXDEFXLT

SEQ ID NO: 37 - protein, 10 aa, synthetic construct
  REGION 1..10: note = synthetic construct
GYTFTTAGMQ

SEQ ID NO: 38 - protein, 10 aa, synthetic construct
  REGION 1..10: note = synthetic construct
GYTFTTAGDQ

SEQ ID NO: 39 - protein, 17 aa, synthetic construct
  REGION 1..17: note = synthetic construct
WINTHSGEPKYAEDFKG

SEQ ID NO: 40 - protein, 17 aa, synthetic construct
  REGION 1..17: note = synthetic construct
WDNTHSGEPKYAEDFKG

SEQ ID NO: 41 - protein, 9 aa, synthetic construct
  REGION 1..9: note = synthetic construct
LQYDEFPLT

SEQ ID NO: 42 - protein, 9 aa, synthetic construct
  REGION 1..9: note = synthetic construct
LQDDEFPLT

SEQ ID NO: 43 - protein, 9 aa, synthetic construct
  REGION 1..9: note = synthetic construct
LQYDEFDLT

SEQ ID NO: 44 - protein, 8 aa, synthetic construct
  REGION 1..8: note = synthetic construct
MGGYAMDY

SEQ ID NO: 45 - protein, 8 aa, synthetic construct
  REGION 1..8: note = synthetic construct
EGGYAMDY

SEQ ID NO: 46 - protein, 8 aa, synthetic construct
  REGION 1..8: note = synthetic construct
MGGYAEDY